(19) United States
(12) Patent Application Publication
(10) Pub. No.: US 2017/0340648 A1
(43) Pub. Date: Nov. 30, 2017

(54) Pharmaceutical Compositions Affecting Bioenergetic Processes in a Eukaryotic Biological System and Methods of Treatment

(71) Applicant: JOYCE BIOTECH CORP., San Juan, PR (US)

(72) Inventor: James Sheehan, Scottsdale, AZ (US)

(73) Assignee: JOYCE BIOTECH CORP., San Juan, PR (US)

(21) Appl. No.: 15/480,885

(22) Filed: Apr. 6, 2017

Related U.S. Application Data
(63) Continuation of application No. 15/163,945, filed on May 25, 2016.

(51) Int. Cl. A61K 31/567; A61K 31/155; A61K 9/00

(52) U.S. Cl. CPC A61K 31/567; A61K 9/0053; A61K 31/155

(57) ABSTRACT

Provided herein are methods for treating diseases and/or disorders and/or medical conditions with pharmaceutical compositions consisting of an association of active principles that affect metabolic processes in a eukaryotic biological system. Also provided herein are methods for the preparation of said pharmaceutical compositions for use in the methods of the embodiments of the present invention. Also provided herein are novel dosing strategies for administering the pharmaceutical compositions that constitute embodiments of the present invention.

### CROSS-REFERENCE TO RELATED APPLICATIONS

This application is a continuation of the utility application Ser. No. 15/163,945 filed May 25, 2016, entitled “Pharmaceutical Compositions Affecting Mitochondrial Redox State and Methods of Treatment”, which is incorporated herein by reference in its entirety.

### FIELD OF THE INVENTION

The present invention is directed to biological compound.

### BACKGROUND OF THE INVENTION

Both prokaryotic and eukaryotic cells depend on a system of bioenergetic metabolism to provide the capacity to do work and maintain cellular integrity amidst the entropic environments in which they exist.

While prokaryotic cells feature a diffuse cytosolic and cell membrane embedded array of enzymes and protein complexes required for such bioenergetic processes, bioenergetic metabolism in eukaryotic cells is distributed between cytosolic domains, membrane embedded domains and distinct membrane defined domains, such as mitochondria.

The bioenergetic processes that drive the production and/or transformation of carriers of free energy in a biological system, namely nucleoside phosphate molecules, such as adenosine triphosphate (ATP), are also the most consistent and/or prolific source of reactive oxygen species (ROS) generation in a eukaryotic biological system.

Many of the most prevalent and emerging sources of morbidity and mortality confronting both the developed and developing worlds are associated with metabolic dysfunction and/or oxidative stress resulting from ROS generation and/or the inability to reduce ROS.

Bioenergetic dysfunction is considered to be a pathoetiological factor in a wide range of diseases and/or disorders and/or conditions including but not limited to: insulin resistance, type 2 diabetes mellitus, cardiovascular disease, renal disease, non-alcoholic fatty liver disease (NAFLD), non-alcoholic steatohepatitis (NASH), neurodegenerative disorders and neoplastic disorders.

Current pharmacological treatment for diseases and/or disorders and/or conditions associated with bioenergetic dysfunction, such as but not limited to; insulin resistance, diabetic cardiomyopathy, the cardio renal metabolic syndrome, cancer, non-alcoholic fatty liver disease (NAFLD), non-alcoholic steatohepatitis (NASH) and neurodegenerative conditions, are not engineered to safely and/or effectively modify the burden of bioenergetic dysfunction and/or oxidative stress, nor the resultant pathological states induced by oxidative stress and/or bioenergetic dysfunction.

Pharmaceutical formulations and methods of use designed to prevent or to treat or to prevent and treat bioenergetic dysfunction associated diseases and/or disorders and/or conditions such as, but not limited to; insulin resistance, neoplastic disorders, non-alcoholic fatty liver disease (NAFLD), non-alcoholic steatohepatitis (NASH), dementia and/or neurodegenerative disorders are desirable and would constitute an advancement of the art.

Accordingly, the embodiments of the present invention provide methods of treatment or methods of prevention or methods of prevention and treatment utilizing pharmaceutical compositions useful in diseases and/or disorders and/or conditions associated with bioenergetic dysfunction in a eukaryotic biological system.

### SUMMARY OF THE INVENTION

Provided herein are novel pharmaceutical compositions and methods to treat or to prevent or to treat and prevent bioenergetic dysfunction associated diseases and/or disorders and/or conditions by effecting bioenergetic processes in an animal, preferably a mammal.

The compositions contain at least two active principal agents and; at least one of the active principal agents is an inhibitor of a mitochondrial process known to generate ROS, and at least one of the other active principals contributes to a reduced rate of mitochondrial oxygen consumption and; the compositions inhibit anaerobic metabolic processes in a eukaryotic biological system.

An active principal agent may demonstrate an ability to both inhibit a mitochondrial process that generates ROS and contribute to a reduced rate of mitochondrial oxygen consumption.

Embodiments of the present invention in the form of pharmaceutical compositions possessing at least two active principal agents where; at least one of the active principal agents is an inhibitor of a mitochondrial process known to generate ROS and; at least one of the other active principals contributes to a reduced rate of mitochondrial oxygen consumption, demonstrate the ability to inhibit anaerobic metabolic processes in a eukaryotic biological system.

Embodiments of the present invention in the form of said pharmaceutical compositions, when administered to a subject in a therapeutically effective amount, possess novel and unexpected emergent properties, namely, molecular structures and/or molecular compounds and/or ionic structures and/or ionic compounds well known in the prior art to increase the rate and/or magnitude of anaerobic metabolic activity and increase the risk of iatrogentic conditions, such as lactic acidosis, when administered to a eukaryotic biological system, paradoxically demonstrate an inhibition of anaerobic metabolic processes when incorporated into pharmaceutical compositions that are embodiments of the present invention.

The observation that the administration, to a subject, of a therapeutically effective amount of an embodiment of the present invention, in the form of pharmaceutical compositions, result in the inhibition of anaerobic metabolic processes in said subject and; that said pharmaceutical compositions can be applied as methods of treatment or methods of prevention or methods of prevention and treatment for diseases or disorders or conditions or diseases and disorders and conditions associated with bioenergetic dysfunction are unexpected considering fundamental concepts of the prior art pertaining to metabolism and pharmacology in a eukaryotic biological system.

### BRIEF DESCRIPTION OF THE DRAWINGS

In the accompanying drawings:

FIG. 1a shows the general molecular structure of metformin.

FIG. 1b shows the general molecular structure of phenformin.

FIG. 1c shows the general molecular structure of buformin.

FIG. 2a shows extracellular flux analysis data comparison of myoblasts treated with Rotenone to the control.

FIG. 2b shows significantly reduced mitochondrial oxygen consumption rate of myoblasts treated with Rotenone compare to control.

FIG. 3a shows observed relative to basal blood lactate levels in a human subject prior/post to Met/Mife treatment.

FIG. 3b shows observed relative to 3 min post exercise blood lactate levels in a human subject prior/post to Met/Mife treatment.

FIG. 3c shows observed relative to 5 min post exercise blood lactate levels in a human subject prior/post to Met/Mife treatment.

FIG. 4a shows significantly increased post exercise blood lactate levels relative to basal blood lactate levels in an untreated human subject.

FIG. 5a shows significantly decreased triglyceride: HDL cholesterol ratio in a human subject post Met/Mife treatment.

FIG. 6a shows significantly decreased serum C reactive protein levels in a human subject post Met/Mife treatment under basal conditions.

FIG. 6b shows significantly decreased serum C reactive protein levels in a human subject post Met/Mife treatment 24 hours post intense physical activity.

FIG. 6c shows significantly decreased serum C reactive protein levels in a human subject post Met/Mife treatment 48 hours post intense physical activity.

FIG. 7a shows significantly decreased urine lipid peroxide concentration in a human subject post Met/Mife treatment 48 hours post intense physical activity.

### BRIEF DESCRIPTION OF THE TABLES

In the accompanying tables:

Table 1A shows Basal ECAR Values (mpH/min) for Control XFAssay_8152014_146.

Table 1B shows Basal ECAR Values (mpH/min) for Metformin (1 mM).

Table 1C shows Basal ECAR Values (mpH/min) for Mifepristone (3 mM).

Table 1D shows Basal ECAR Values (mpH/min) for Metformin/Mifepristone (1 mM/3 mM).

Table 2A shows Basal OCR Values (pMoles/min) for Control XFAssay_8152014_146.

Table 2B shows Basal OCR Values (pMoles/min) for Metformin (1 mM).

Table 2C shows Basal OCR Values (pMoles/min) for Mifepristone (3 mM).

Table 2D shows Basal OCR Values (pMoles/min) for Metformin/Mifepristone (1 mM/3 mM).

Table 3A shows Oligomycin Exposure ECAR Values (mpH/min) for Control XFAssay_8152014_146.

Table 3B shows Oligomycin Exposure ECAR Values (mpH/min) for Metformin 1 mM.

Table 3C shows Oligomycin Exposure ECAR Values (mpH/min) for Mifepristone 3 mM.

Table 3D shows Oligomycin Exposure ECAR Values (mpH/min) for Metformin/Mifepristone 1 mM/3 mM.

Table 4A shows Oligomycin Exposure OCR Values (pMoles/min) for Control XFAssay_8152014_146.

Table 4B shows Oligomycin Exposure OCR Values (pMoles/min) for Metformin (1 mM).

Table 4C shows Oligomycin Exposure OCR Values (pMoles/min) for Mifepristone (3 mM).

Table 4D shows Oligomycin Exposure OCR Values (pMoles/min) for Metformin/Mifepristone (1 mM/3 mM).

Table 5A shows FCCP Exposure ECAR Values (mpH/min) for Control XFAssay_8152014_146.

Table 5B shows FCCP Exposure ECAR Values (mpH/min) for Metformin (1 mM).

Table 5C shows FCCP Exposure ECAR Values (mpH/min) for Mifepristone (3 mM).

Table 5D shows FCCP Exposure ECAR Values (mpH/min) for Metformin/Mifepristone (1 mM/3 mM).

Table 6A shows FCCP Exposure OCR Values (pMoles/min) for Control.

Table 6B shows FCCP Exposure OCR Values (pMoles/min) for Metformin (1 mM).

Table 6C shows FCCP Exposure OCR Values (pMoles/min) for Mifepristone (3 mM) XFAssay_8152014_146.

Table 6D shows FCCP Exposure OCR Values (pMoles/min) for Metformin/Mifepristone (1 mM/3 mM) XFAssay_8152014_146.

Table 7A shows Rotenone Exposure ECAR Values (mpH/min) for Control.

Table 7B shows Rotenone Exposure ECAR Values (mpH/min) for Metformin (1 mM).

Table 7C shows Rotenone Exposure ECAR Values (mpH/min) for Mifepristone (3 mM).

Table 7D shows Rotenone Exposure ECAR Values (mpH/min) for Metformin/Mifepristone (1 mM/3 mM).

Table 8A shows Rotenone Exposure OCR Values (pMoles/min) for Control.

Table 8B shows Rotenone Exposure OCR Values (pMoles/min) for Metformin (1 mM).

Table 8C shows Rotenone Exposure OCR Values (pMoles/min) for Mifepristone (3 mM).

Table 8D shows Rotenone Exposure OCR Values (pMoles/min) for Metformin/Mifepristone (1 mM/3 mM).

Table 9A shows Basal ECAR Values (mpH/min) for Control XFAssay_8222014_853.

Table 9B shows Basal ECAR Values (mpH/min) for Metformin (1 mM).

Table 9C shows Basal ECAR Values (mpH/min) for Mifepristone (50 μM).

Table 9D shows Basal ECAR Values (mpH/min) for Metformin/Mifepristone (1 mM/50 μM).

Table 10A shows Basal OCR Values (pMoles/min) for Control XFAssay_8222014_853.

Table 10B shows Basal OCR Values (pMoles/min) for Metformin (1 mM).

Table 10C shows Basal OCR Values (pMoles/min) for Mifepristone (50 μM).

Table 10D shows Basal OCR Values (pMoles/min) for Metformin/Mifepristone (1 mM/50 μM).

Table 11A shows Oligomycin Exposure ECAR Values (mpH/min) for Control XFAssay_8222014_853.

Table 11B shows Oligomycin Exposure ECAR Values (mpH/min) for Metformin (1 mM).

Table 11C shows Oligomycin Exposure ECAR Values (mpH/min) for Mifepristone (50 μM).

Table 11D shows Oligomycin Exposure ECAR Values (mpH/min) for Metformin/Mifepristone (1 mM/50 μM).

Table 12A shows Oligomycin Exposure OCR Values (pMoles/min) for Control.

Table 12B shows Oligomycin Exposure OCR Values (pMoles/min) for Metformin (1 mM).

Table 12C shows Oligomycin Exposure OCR Values (pMoles/min) for Mifepristone.

Table 12D shows Oligomycin Exposure OCR Values (pMoles/min) for Metformin/Mifepristone (1 mM/50 μM).

Table 13A shows FCCP Exposure ECAR Values (mpH/min) for Control XFAssay_8222014_853.

Table 13B shows FCCP Exposure ECAR Values (mpH/min) for Metformin (1 mM).

Table 13C shows FCCP Exposure ECAR Values (mpH/min) for Mifepristone (50 μM).

Table 13D shows FCCP Exposure ECAR Values (mpH/min) for Metformin/Mifepristone (1 mM/50 μM).

Table 14A shows FCCP Exposure OCR Values (pMoles/min) for Control.

Table 14B shows FCCP Exposure OCR Values (pMoles/min) for Metformin (1 mM).

Table 14C shows FCCP Exposure OCR Values (pMoles/min) for Mifepristone (50 μM).

Table 14D shows FCCP Exposure OCR Values (pMoles/min) for Metformin/Mifepristone (1 mM/50 μM).

Table 15A shows Rotenone Exposure ECAR Values (mpH/min) for Control.

Table 15B shows Rotenone Exposure ECAR Values (mpH/min) for Metformin (1 mM).

Table 15C shows Rotenone Exposure ECAR Values (mpH/min) for Mifepristone (50 μM).

Table 15D shows Rotenone Exposure ECAR Values (mpH/min) for Metformin/Mifepristone (1 mM/50 μM) XFAssay_8222014_853.

Table 16A shows Rotenone Exposure OCR Values (pMoles/min) for Control.

Table 16B shows Rotenone Exposure OCR Values (pMoles/min) for Metformin (1 mM).

Table 16C shows Rotenone Exposure OCR Values (pMoles/min) for Mifepristone (50 μM).

Table 16D shows Rotenone Exposure OCR Values (pMoles/min) for Metformin/Mifepristone (1 mM/50 μM) XFAssay_8222014_853.

Table 17A shows Basal ECAR Values (mpH/min) for Control XFAssay_10232014_839.

Table 17B shows Basal ECAR Values (mpH/min) for Metformin (25 μM).

Table 17C shows Basal ECAR Values (mpH/min) for Mifepristone (50 μM).

Table 17D shows Basal ECAR Values (mpH/min) for Metformin/Mifepristone (25 μM/50 μM).

Table 18A shows Basal OCR Values (pMoles/min) for Control XFAssay_10232014_839.

Table 18B shows Basal OCR Values (pMoles/min) for Metformin (25 μM).

Table 18C shows Basal OCR Values (pMoles/min) for Mifepristone (50 μM).

Table 18D shows Basal OCR Values (pMoles/min) for Metformin/Mifepristone (25 μM/50 μM).

Table 19A shows Oligomycin Exposure ECAR Values (mpH/min) for Control.

Table 19B shows Oligomycin Exposure ECAR Values (mpH/min) for Metformin (25 μM).

Table 19C shows Oligomycin Exposure ECAR Values (mpH/min) for Mifepristone (50 μM).

Table 19D shows Oligomycin Exposure ECAR Values (mpH/min) for Metformin/Mifepristone (25 μM/50 μM) XFAssay_10232014_839.

Table 20A shows Oligomycin OCR Values (pMoles/min) for Control.

Table 20B shows Oligomycin OCR Values (pMoles/min) for Metformin (25 μM).

Table 20C shows Oligomycin OCR Values (pMoles/min) for Mifepristone (50 μM).

Table 20D shows Oligomycin OCR Values (pMoles/min) for Metformin/Mifepristone (25 μM/50 μM).

Table 21A shows FCCP Exposure ECAR Values (mpH/min) for Control.

Table 21B shows FCCP Exposure ECAR Values (mpH/min) for Metformin (25 μM).

Table 21C shows FCCP Exposure ECAR Values (mpH/min) for Mifepristone (50 μM).

Table 21D shows FCCP Exposure ECAR Values (mpH/min) for Metformin/Mifepristone (25 μM/50 μM).

Table 22A shows FCCP OCR Values (pMoles/min) for Control XFAssay_10232014_839.

Table 22B shows FCCP OCR Values (pMoles/min) for Metformin (25 μM).

Table 22C shows FCCP OCR Values (pMoles/min) for Mifepristone (50 μM).

Table 22D shows FCCP OCR Values (pMoles/min) for Metformin/Mifepristone (25 μM/50 μM) XFAssay_10232014_839.

Table 23A shows Rotenone Exposure ECAR Values (mpH/min) for Control.

Table 23B shows Rotenone Exposure ECAR Values (mpH/min) for Metformin (25 μM).

Table 23C shows Rotenone Exposure ECAR Values (mpH/min) for Mifepristone (50 μM).

Table 23D shows Rotenone Exposure ECAR Values (mpH/min) for Metformin/Mifepristone (25 μM/50 μM) XFAssay_10232014_839.

Table 24A shows Rotenone OCR Values (pMoles/min) for Control XFAssay_10232014_839.

Table 24B shows Rotenone OCR Values (pMoles/min) for Metformin (25 μM).

Table 24C shows Rotenone OCR Values (pMoles/min) for Mifepristone (50 μM).

Table 24D shows Rotenone OCR Values (pMoles/min) for Metformin/Mifepristone (25 μM/50 μM) XFAssay_10232014_839.

Table 25A shows Composite Basal-ECAR MET (1 mM)+Basal-ECAR MIFE (3 mM).

Table 25B shows Composite Basal-OCR MET (1 mM)+Basal-ECAR (3 mM).

Table 25C shows Composite FCCP-ECAR MET (1 mM)+FCCP-ECAR (3 mM).

Table 25D shows Composite FCCP-OCR MET (1 mM)+FCCP-OCR (3 mM).

Table 25E shows Composite Basal-ECAR MET (1 mM)+Basal-ECAR MIFE (50 uM).

Table 25F shows Composite Basal-OCR MET (1 mM)+Basal-OCR MIFE (50 uM).

Table 25G shows Composite FCCP-ECAR MET (1 mM)+FCCP-ECAR MIFE (50 uM).

Table 25H shows Composite FCCP-OCR MET (1 mM)+FCCP-OCR MIFE (50 uM).

Table 25I shows Composite Basal-ECAR MET (25 uM)+Basal-ECAR MIFE (50 uM).

Table 25J shows Composite Basal-OCR MET (25 uM)+Basal-OCR MIFE (50 uM).

Table 25K shows Composite FCCP-ECAR MET (25 uM)+FCCP-ECAR MIFE (50 uM).

Table 25L shows Composite FCCP-OCR MET (25 uM)+FCCP-OCR MIFE (50 uM).

Table 26A shows Basal-ECAR Control XFAssay_712015_1658.

Table 26B shows Basal-ECAR KET (1 mM) XFAssay_712015_1658.

Table 26C shows Basal-ECAR KET (50 uM) XFAssay_712015_1658.

Table 26D shows Basal-ECAR MET/KET (1 mM/50 uM) XFAssay_712015_1658.

Table 26E shows Basal-OCR Control XFAssay_712015_1658.

Table 26F shows Basal-OCR KET (1 mM) XFAssay_712015_1658.

Table 26G shows Basal-OCR KET (50 uM) XFAssay_712015_1658.

Table 26H shows Basal-OCR MET/KET (1 mM/50 uM) XFAssay_712015_1658.

Table 26I shows Oligomycin-ECAR Control XFAssay_712015_1658.

Table 26J shows Oligomycin-ECAR KET (1 mM) XFAssay_712015_1658.

Table 26K shows Oligomycin-ECAR KET (50 uM) XFAssay_712015_1658.

Table 26L shows Oligomycin-ECAR MET/KET (1 mM/50 uM) XFAssay_712015_1658.

Table 26M shows Oligomycin-OCR Control XFAssay_712015_1658.

Table 26N shows Oligomycin-OCR KET (1 mM) XFAssay_712015_1658.

Table 26O shows Oligomycin-OCR KET (50 uM) XFAssay_712015_1658.

Table 26P shows Oligomycin-OCR MET/KET (1 mM/50 uM) XFAssay_712015_1658.

Table 26Q shows FCCP-ECAR Control XFAssay_712015_1658.

Table 26R shows FCCP-ECAR KET (1 mM) XFAssay_712015_1658.

Table 26S shows FCCP-ECAR KET (50 uM) XFAssay_712025_1658.

Table 26T shows FCCP-ECAR MET/KET (1 mM/50 uM) XFAssay_712015_1658.

Table 26U shows FCCP-OCR Control XFAssay_712015_1658.

Table 26V shows FCCP-OCR KET (1 mM) XFAssay_712015_1658.

Table 26W shows FCCP-OCR KET (50 uM) XFAssay_712015_1658.

Table 26X shows FCCP-OCR MET/KET (1 mM/50 uM) XFAssay_712015_1658.

Table 26Y shows Rotenone-ECAR Control XFAssay_712015_1658.

Table 26Z shows Rotenone-ECAR KET (1 mM) XFAssay_712015_1658.

Table 26Z1 shows Rotenone-ECAR KET (50 uM) XFAssay_712015_1658.

Table 26Z2 shows Rotenone-ECAR MET/KET (1 mM/50 uM) XFAssay_712015_1658.

Table 26Z3 shows Rotenone-OCR Control XFAssay_712015_1658.

Table 26Z4 shows Rotenone-OCR KET (1 mM) XFAssay_712015_1658.

Table 26Z5 shows Rotenone-OCR KET (50 uM) XFAssay_712015_1658.

Table 26Z6 shows Rotenone-OCR MET/KET (1 mM/50 uM) XFAssay_712015_1658.

Table 27A1 shows Basal-ECAR Control Vs. Basal-ECAR MET/MIFE (25 uM/50 uM).

Table 27A2 shows Basal-ECAR Control Vs. Basal-ECAR Rotenone.

Table 27A3 shows Basal-ECAR Rotenone Vs. Basal-ECAR MET/MIFE (25 uM/50 uM).

Table 27A4 shows Basal-OCR Control Vs. Basal-OCR MET/MIFE (25 uM/50 uM).

Table 27A5 shows Basal-OCR Control Vs. Basal-OCR Rotenone.

Table 27A6 shows Basal-OCR Rotenone Vs. Basal-OCR MET/MIFE (25 uM/50 uM).

Table 27A7 shows FCCP-ECAR Control Vs. FCCP-ECAR MET/MIFE (25 uM/50 uM).

Table 27A8 shows FCCP-OCR Control Vs. FCCP-OCR MET/MIFE (25 uM/50 uM).

Table 27B1 shows Basal-ECAR Control Vs. Basal-ECAR MET/MIFE (1 mM/50 uM).

Table 27B2 shows Basal-ECAR Control Vs. Basal-ECAR Rotenone.

Table 27B3 shows Basal-ECAR Rotenone Vs. Basal-ECAR MET/MIFE (1 mM/50 uM).

Table 27B4 shows Basal-OCR Control Vs. Basal-OCR MET/MIFE (1 mM/50 uM).

Table 27B5 shows Basal-OCR Control Vs. Basal-OCR Rotenone.

Table 27B6 shows Basal-OCR Rotenone Vs. Basal-OCR MET/MIFE (1 mM/50 uM).

Table 27B7 shows Ratio of Basal-ECAR/Basal-OCR for Rotenone Vs. Ratio of Basal-ECAR/Basal-OCR for Met/Mife (1 mM/50 uM) XFAssay_8222014_853.

Table 27B8 shows FCCP-ECAR Control Vs. FCCP-ECAR MET/MIFE (1 mM/50 uM).

Table 27B9 shows FCCP-OCR Control Vs. FCCP-OCR MET/MIFE (1 mM/50 uM).

Table 27C1 shows Basal-ECAR Control Vs. Basal-ECAR MET/MIFE (1 mM/3 mM).

Table 27C2 shows Basal-ECAR Control Vs. Basal-ECAR Rotenone.

Table 27C3 shows Basal-ECAR Rotenone Vs. Basal-ECAR MET/MIFE (1 mM/3 mM).

Table 27C4 shows Basal-OCR Control Vs. Basal-OCR MET/MIFE (1 mM/3 mM).

Table 27C5 shows Basal-OCR Control Vs. Basal-OCR Rotenone.

Table 27C6 shows Basal-OCR Rotenone Vs. Basal-OCR MET/MIFE (1 mM/3 mM).

Table 27C7 shows Ratio of Basal-ECAR/Basal-OCR for Rotenone Vs. Ratio of Basal-ECAR/Basal-OCR for Met/Mife (1 mM/3 mM) XFAssay_8152014_146.

Table 27C8 shows FCCP-ECAR Control Vs. FCCP-ECAR MET/MIFE (1 mM/3 mM).

Table 27C9 shows FCCP-OCR Control Vs. FCCP-OCR MET/MIFE (1 mM/3 mM).

Table 27D1 shows Basal-ECAR Composite Mean MET (1 mM)+MIFE (3 mM) Vs. Basal-ECAR MET/MIFE (1 mM/3 mM).

Table 27D2 shows Basal-OCR Composite Mean MET (1 mM)+MIFE (3 mM) Vs. Basal-OCR MET/MIFE (1 mM/3 mM).

Table 27D3 shows FCCP-ECAR Composite Mean MET (1 mM)+MIFE (3 mM) Vs. FCCP-ECAR MET/MIFE (1 mM/3 mM).

Table 27D4 shows FCCP-OCR Composite Mean MET (1 mM)+MIFE (3 mM) Vs. FCCP-OCR MET/MIFE (1 mM/3 mM).

Table 27D5 shows Basal-ECAR Composite MET (1 mM)+MIFE (50 uM) Vs. Basal-ECAR MET/MIFE (1 mM/50 uM).

Table 27D6 shows Basal-OCR Composite MET (1 mM)+MIFE (50 uM) Vs. Basal-OCR MET/MIFE (1 mM/50 uM).

Table 27D7 shows FCCP-ECAR Composite MET (1 mM)+MIFE (50 uM) Vs. FCCP-ECAR MET/MIFE (1 mM/50 uM).

Table 27D8 shows FCCP-OCR Composite MET (1 mM)+MIFE (50 uM) Vs. FCCP-OCR MET/MIFE (1 mM/50 uM).

Table 27D9 shows Basal-ECAR Composite MET (25 uM)+MIFE (50 uM) Vs. Basal-ECAR MET/MIFE (25 uM/50 uM).

Table 27D10 shows Basal-OCR Composite MET (25 uM)+MIFE (50 uM) Vs. Basal-OCR MET/MIFE (25 uM/50 uM).

Table 27D11 shows FCCP-ECAR Composite MET (25 uM)+MIFE (50 uM) Vs. FCCP-ECAR MET/MIFE (25 uM/50 uM).

Table 27D12 shows FCCP-OCR Composite MET (25 uM)+MIFE (50 uM) Vs. FCCP-OCR MET/MIFE (25 uM/50 uM).

Table 27E1 shows FCCP-ECAR Control Vs. FCCP-ECAR MET/KET (1 mM/50 uM).

Table 27E2 shows FCCP-OCR Control Vs. FCCP-OCR MET/KET (1 mM/50 uM)).

Table 27F1 shows The Ratio of Basal-ECAR/Basal-OCR MET/KET (1 mM/50 uM) Vs. The Ratio of Basal-ECAR/Basal-OCR MET/MIFE (1 mM/50 uM).

Table 27F2 shows The Ratio of FCCP-ECAR/FCCP-OCR MET/KET (1 mM/50 uM) Vs. The Ratio of FCCP-ECAR/FCCP-OCR MET/MIFE (1 mM/50 uM).

Table 27F3 shows Basal-ECAR Control Vs. Basal-ECAR MET/KET (1 mM/50 uM).

Table 28A shows Experiment 2 Baseline Body Composition.

Table 28B shows Experiment 2 Baseline Laboratory Analysis-Blood.

Table 28C shows Experiment 2 Baseline Laboratory Analysis-Urine.

Table 28D shows Baseline Exercise To Exhaustion Test Protocol.

Table 28E shows 24-Hr Post-Baseline Exercise To Exhaustion Test Protocol Lab. analysis-Blood.

Table 28F shows 48-Hour Post-Baseline Exercise To Exhaustion Test Protocol Laboratory Analysis-Blood & Urine.

Table 28G shows Experiment 2 Post-Metformin/Mifepristone Treatment Lab. Analysis-Blood.

Table 28G1 shows Experiment 2 Post-Treatment Laboratory Analysis-Urine.

Table 28H shows 24-Hr Post-Baseline Exercise To Exhaustion Test Protocol Lab. Analysis-Blood.

Table 28H1 shows 48-Hr Post-Treatment Exercise To Exhaustion Test Protocol Lab. Analysis-Blood & Urine.

Table 28I shows Experiment 2 Post-Metformin/Mifepristone Treatment Body Composition.

Table 28J shows Experiment 2 Post-Metformin/Mifepristone Treatment Exercise To Exhaustion Test Protocol.

### Definitions of Terms Used in Descriptions

The below terms as used in this application are intended to have the meaning defined below, as the definition would be understood by persons of typical experience in the chemical or biological or chemical and biological arts. The use or non-use of capitalization in this application is not to impart any difference in whether the term as defined is intended.

The abbreviations used herein have their conventional meaning within the chemical and biological arts.

The term “Acetyl Coenzyme A” or “Acetyl CoA” or “Coenzyme A” or “acetyl-CoA” or “CoASH” as used herein, means a molecule that participates in many biochemical reactions in protein and/or carbohydrate and/or lipid metabolism. The main function of Acetyl CoA is to deliver the acetyl group to the citric acid cycle (Krebs cycle) to be oxidized for metabolic energy production. Coenzyme A or CoASH or CoA consists of a Beta-mercaptoethylamine linked to the vitamin pantothenic acid through an amide linkage. The acetyl group of acetyl-CoA is linked to the sulfhydryl substituent of the β-mercaptoethylamine group. This thioester linkage is a “high energy” bond, which is particularly reactive. Hydrolysis of the thioester bond is exergonic. CoA is acetylated during breakdown of carbohydrates through glycolysis, as well as by the beta-oxidation of fatty acids. It then enters the citric acid cycle, where the acetyl group is further oxidized to carbon dioxide and water, with the energy thus released captured in the form of 11 ATP and 1 GTP per acetyl group. Acetyl-CoA is produced during cellular respiration during the Swanson Conversion, which takes place in the mitochondria of eukaryotic bioenergetic systems. After the Swanson Conversion the acetyl-CoA is moved into Krebs Cycle.

The term “active principal” or “active agent” or “principal agent” or “active principal agent”, as used herein, means a molecular compound and/or molecular structure and/or ionic compound and/or ionic structure and/or the metabolites of a molecular compound and/or the metabolites of a molecular structure and/or the metabolites of an ionic compound and/or the metabolites of an ionic structure and/or prodrugs and/or conjugates and/or other such derivatives, analogs and/or related compounds that through interaction with a biological system directly and/or indirectly results in an alteration of said biological system.

The term “aerobic metabolism” or “aerobic pathway” or “aerobic”, as used herein, means the process of transforming metabolic substrate, including but not limited to Acetyl Coenzyme A into nucleoside phosphate molecules that requires oxygen and includes the molecular compounds and/or molecular structures and/or ionic compounds and/or ionic structures and/or molecular compounds required by a mitochondrion in a eukaryotic biological system for the transformation of metabolic substrate into nucleoside phosphate molecules. Additionally, the term “aerobic metabolism” or “aerobic pathway” or “aerobic” includes the autocrine and/or endocrine and/or neurological and/or immunological and/or genetic structures and/or actions which regulate the process of “aerobic metabolism”, as well as the organelles and/or cells and/or tissues and/or organs and/or organ systems and/or organisms and/or routes of transport (including but not limited to circulatory and/or lymphatic) utilized by the biological system in the construction and/or maintenance and/or regulation of “aerobic metabolism”.

The term “allopurinol” or “1,2 Dihydropyrazolo(3,4-d)pyrimidin-4-one”, as used herein, means molecular compounds or molecular structures or molecular compounds and molecular structures that are described by simplified molecular-input line-entry system (SMILES)as; C1=C2C(═NC═NC2=O)NN1 and; metabolites and/or prodrugs and/or conjugates and/or other such derivatives and/or analogs and/or related compounds of the molecular structure and/or the molecular compound described by simplified molecular-input line-entry system (SMILES)as; C1=C2C(═NC═NC2=O)NN1 or oxypurinol or “1,2 Dihydropyrazolo(3,4-d)pyrimidine-4,6-dione” or molecular compounds or molecular structures or molecular compounds and molecular structures that are described by simplified molecular-input line-entry system (SMILES)as; C1=C2C(═NC(═O)NC2=O)NN1 and; metabolites and/or prodrugs and/or conjugates and/or other such derivatives and/or analogs and/or related compounds of the molecular structure and/or the molecular compound described by simplified molecular-input line-entry system (SMILES)as; C1=C2C(═NC(═O)NC2=O)NN1.

The term “anaerobic metabolism” or “anaerobic pathway” or “anaerobic” as used herein, means the process of transforming metabolic substrate, including but not limited to glucose, into nucleoside phosphate molecules and/or metabolic substrate including but not limited to Acetyl Coenzyme A and/or pyruvate that does not require oxygen nor mitochondria and; includes but is not limited to the phosphate system and/or glycolysis and includes the molecular compounds and/or molecular structures and/or ionic compounds and/or ionic structures required by a biological system for the “anaerobic” transformation of metabolic substrate into nucleoside phosphate molecules and/or metabolic substrate including but not limited to Acetyl Coenzyme A and/or pyruvate. Additionally, term “anaerobic metabolism” or “anaerobic pathway” or “anaerobic” includes the autocrine and/or endocrine and/or neurological and/or immunological and/or genetic structures and/or actions which regulate the process of “anaerobic metabolism”, as well as the organelles and/or cells and/or tissues and/or organs and/or organ systems and/or organisms and/or routes of transport (including but not limited to circulatory and/or lymphatic transport) utilized by the biological system in the construction and/or maintenance and/or regulation of “anaerobic metabolism”.

The term “ATP synthase” or “F₀ F₁ ATP synthase” or “complex V”, as used herein, means an enzyme possessing the properties necessary to catalyze a chemical process defined by Enzyme Commission number EC 3.6.3.14 and includes the autocrine or endocrine or neurological or immunological or genetic structures or action or structures and action or autocrine and endocrine and neurological and immunological and genetic structures or actions or structures and actions which regulates “ATP synthase”, as well as the organelles or cells or tissues or organs or organ systems or organisms or organelles and cells and tissues and organs and organ systems and organisms and routes of transport (including but not limited to circulatory or lymphatic or circulatory and lymphatic transport) utilized by a biological system in the construction and/or maintenance and/or regulation of “ATP synthase”.

The term “bioenergetic” or “bioenergetic metabolism”, as used herein, means the process of transforming metabolic substrate into nucleoside phosphate molecules and includes but is not limited to; aerobic metabolism and anaerobic metabolism. Additionally the term “bioenergetic” or “bioenergetic metabolism” includes the molecular compounds and/or molecular structures and/or ionic compounds and/or ionic structures and/or molecular compounds required by a eukaryotic biological system for the transformation of metabolic substrate into nucleoside phosphate molecules and includes the autocrine and/or endocrine and/or neurological and/or immunological and/or genetic structures and/or actions which regulate the process of “bioenergetic metabolism”, as well as the organelles and/or cells and/or tissues and/or organs and/or organ systems and/or organisms and/or routes of transport (including but not limited to circulatory and/or lymphatic) utilized by the biological system in the construction and/or maintenance and/or regulation of “bioenergetic metabolism”.

The term “bioenergetic dysfunction” or “bioenergetic metabolic dysfunction”, as used herein, means the sub-optimal functioning and/or functioning that does not support homoestasis of the process of transforming metabolic substrate into nucleoside phosphate molecules and includes but is not limited to; aerobic metabolism and anaerobic metabolism. Additionally the term “bioenergetic dysfunction” or “bioenergetic metabolic dysfunction” includes the sub-optimal functioning and/or functioning that does not support homoestasis of molecular compounds and/or molecular structures and/or ionic compounds and/or ionic structures and/or molecular compounds required by a eukaryotic biological system for the transformation of metabolic substrate into nucleoside phosphate molecules and includes the sub-optimal functioning and/or functioning that does not support homoestasis of autocrine and/or endocrine and/or neurological and/or immunological and/or genetic structures and/or actions which regulate the process of “bioenergetic metabolism”, as well as the sub-optimal functioning and/or functioning that does not support homoestasis of organelles and/or cells and/or tissues and/or organs and/or organ systems and/or organisms and/or routes of transport (including but not limited to circulatory and/or lymphatic) utilized by the biological system in the construction and/or maintenance and/or regulation of “bioenergetic metabolism”.

The term “biological system”, as used herein, means molecular compounds and/or molecular structures and/or ionic compounds and/or ionic structures and/or molecular compounds that constitute a system of organization that through a minimum level of structure and/or action is able to resist entropic forces and maintain said organization through homeostatic measures, including but not limited to aerobic and/or anaerobic metabolism. Additionally, the term “biological system” includes the autocrine and/or endocrine and/or neurological and/or immunological and/or genetic structures and/or actions which regulate the homeostatic measures of a “biological system”, as well as the organelles and/or cells and/or tissues and/or organs and/or organ systems and/or organisms and/or routes of transport (including but not limited to circulatory and/or lymphatic) utilized by the biological system in the construction and/or maintenance and/or regulation of homeostatic measures in a “biological system”.

The term “buformin” or “1-butylbiguanide” or “2-butyl-1-(diaminomethylidene) guanidine”, as used herein, means molecular compounds and/or molecular structures that are described by simplified molecular-input line-entry system (SMILES) as; CCCCNC(═N)NC(═N)N and; metabolites and/or prodrugs and/or conjugates and/or other such derivatives and/or analogs and/or related compounds of the molecular structure and/or the molecular compound described by simplified molecular-input line-entry system (SMILES)as; CCCCNC(═N)NC(═N)N.

The term “complex I” or “NADH: ubiquinone reductase” or “Coenzyme Q reductase”, as used herein, means an enzyme possessing the properties necessary to catalyze a chemical process defined by Enzyme Commission number EC 1.6.5.3 and includes the autocrine or endocrine or neurological or immunological or genetic structures or actions or structures and actions or autocrine and endocrine and neurological and immunological and genetic structures or actions or structures and actions which regulate “complex I”, as well as the organelles or cells or tissues or organs or organ systems or organisms or organelles and cells and tissues and organs and organ systems and organisms and routes of transport (including but not limited to circulatory or lymphatic or circulatory and lymphatic transport) utilized by the biological system in the construction or maintenance or construction and maintenance of “complex I”.

The term “complex II” or “Succinate dehydrogenase” or “Succinate: quinone oxidoreductase”, as used herein, means an enzyme possessing the properties necessary to catalyze a chemical process defined by Enzyme Commission number EC 1.3.5.1 and includes the autocrine or endocrine or neurological or immunological or genetic structures or actions or structures and actions or autocrine and endocrine and neurological and immunological and genetic structures or actions or structures and actions which regulate “complex II”, as well as the organelles or cells or tissues or organs or organ systems or organisms or organelles and cells and tissues and organs and organ systems and organisms and routes of transport (including but not limited to circulatory or lymphatic or circulatory and lymphatic transport) utilized by the biological system in the construction or maintenance or construction and maintenance of “complex II”.

The term “complex III” or “Ubiquinol cytochrome-c reductase” or “Quinol cytochrome-c reductase”, as used herein, means an enzyme possessing the properties necessary to catalyze a chemical process defined by Enzyme Commission number EC 1.10.2.2 and includes the autocrine or endocrine or neurological or immunological or genetic structures or actions or structures and actions or autocrine and endocrine and neurological and immunological and genetic structures or actions or structures and actions which regulate “complex III”, as well as the organelles or cells or tissues or organs or organ systems or organisms or organelles and cells and tissues and organs and organ systems and organisms and routes of transport (including but not limited to circulatory or lymphatic or circulatory and lymphatic transport) utilized by the biological system in the construction or maintenance or construction and maintenance of “complex III”.

The term “cytosolic high energy phosphate system (CHEPS)” or “cytosolic high energy system” or “phosphagen system”, as used herein, means the process of maintaining a ratio of the cytosolic concentration of phosphagen energy storage compounds (including but not limited to; phosphocreatine or arginine phosphate or phospholombricine) to the cytosolic concentration of free energy as characterized by nucleoside triphosphate concentration (including, but not limited to, adenosine triphosphate and guanosine-5′-triphosphate) or the process of maintaining a ratio of the cytosolic concentration of phosphagen energy storage compounds to the cytosolic concentration of free inorganic phosphate or the process of maintaining a ratio of the cytosolic concentration of phosphagen energy storage compounds to the cytosolic concentration of nucleoside triphosphate concentration and the process of maintaining a ratio of the cytosolic concentration of phosphagen energy storage compounds to the cytosolic concentration of free inorganic phosphate and includes the molecular compounds or molecular structures or ionic compounds or ionic structures or molecular compounds and molecular structures and ionic compounds and ionic structures required for the process of maintaining a ratio of the cytosolic concentration of phospahagen energy storage compounds to the cytosolic concentration of nucleoside triphosphate concentration or the cytosolic concentration of phosphagen energy storage compounds to the cytosolic concentration of free inorganic phosphate or the cytosolic concentration of phosphagen energy storage compounds to the cytosolic concentration of nucleoside triphosphate concentration and the cytosolic concentration of phosphagen energy storage compounds to cytosolic free inorganic phosphate. Additionally, it includes the autocrine or endocrine or neurological or immunological or genetic structures or actions or structures and actions or the autocrine and endocrine and neurological and immunological and genetic structures or actions or structures and actions which regulate the cytosolic high energy phosphate system, as well as the organelles or cells or tissues or organs or organ systems or organisms or organelles and cells and tissues and organs and organ systems and organisms and routes of transport (including but not limited to circulatory or lymphatic or circulatory and lymphatic transport) utilized by the biological system in the construction or maintenance or the construction and maintenance of the “cytosolic high energy phosphate system”.

The term “condition” or “medical condition”, as used herein, means the state of a biological system or the state of an element of a biological system or the state of a biological system and the state of an element of a biological system that as a result of structures or actions or structures and actions predisposes said biological system or said element of a biological system or said biological system and said element of a biological system to disease or disorder or disease and disorder and a “condition” may constitute an element of disease or disorder or disease and disorder.

The term “disease”, as used herein, means the state of a biological system or the state of an element of a biological system or the state of a biological system and the state of an element of a biological system that as a result of structures or actions or structures and actions satisfies diagnostic criteria as defined by the art of the field as interpreted by those skilled in the art.

The term “disorder” or “medical disorder”, as used herein, means the state of a biological system or the state of an element of a biological system or the state of a biological system and the state of an element of a biological system that as a result of structures or actions or structures and actions deviates from the desired state of said biological system or the desired state of said element of a biological system or the desired state of said biological system and said element of a biological system with or without satisfying specific diagnostic criteria as defined by the art of the field as interpreted by those skilled in the art.

The term “emergent properties”, as used herein, means the effects a composition exerts on structures or actions or structures and actions that are novel or non-additive or novel and non-additive in comparison to the effects exerted by the constituent elements of said composition on structures or actions or structures and actions and includes the magnitude of the effects or the physical nature of the effects or the temporal nature of the effects or the spatial nature of the effects or the magnitude of the effects and the physical nature of the effects and the temporal nature of the effects and the nature of the effects.

The term “glycolysis” or “glycolytic pathway” or “anaerobic glycolysis” as used herein, means the metabolic pathway that converts glucose C₆H₁₂O₆, into, pyruvate (CH₃COCOO⁻+H⁺). The free energy released as a result of glycolysis is used to form adenosine triphosphate (ATP) and reduced nicotinamide adenine dinucleotide (NADH). The intermediate enzymatic steps of glycolysis provide entry points for metabolic substrate including but not limited to monosaccharides, such as fructose and/or galactose, which can be converted to one of these intermediates. The intermediates may also be directly useful, such as the intermediate dihydroxyacetone dihydroxyacetone (DHAP), a source of the glycerol that combines with fatty acids to form triglycerides. Glycolysis is an anaerobic metabolic pathway, meaning that it does not use molecular oxygen for any of its reactions. However the products of glycolysis (pyruvate and NADH+H⁺) may be further metabolized via aerobic metabolic pathways. Glycolysis occurs, with some variations, in nearly every biological system. Glycolysis occurs in most biological systems in the cytosol of the biological system. The term glycolysis means pathways including but not limited to the Embden-Meyerhof-Parnas (EMP pathway) and/or the Entner-Doudoroff pathway and/or various heterofermentative and homofermentative pathways. Additionally, the term “glycolysis” includes the autocrine or endocrine or neurological or immunological or genetic structures or actions or structures and actions or the autocrine and endocrine and neurological and immunological and genetic structures or actions or structures and actions which regulate glycolysis, as well as the organelles or cells or tissues or organs or organ systems or organisms or organelles and cells and tissues and organs and organ systems and organisms and routes of transport (including but not limited to circulatory or lymphatic or circulatory and lymphatic transport) utilized by the biological system in the construction or maintenance or the construction and maintenance of glycolysis.

The term “homeostatic measures”, as used herein, means structures or actions or structures and actions employed by a biological system or elements of a biological system or a biological system and elements of a biological system in an effort to maintain a given variable or multiple variables or a given variable and multiple variables within an intended range.

The term “intermembrane space” or “IMS” or “mitochondrial intermembrane space”, as used herein, means a space that may or may not contain molecular compounds or molecular structures or ionic compounds or ionic structures or molecular compounds and molecular structures and ionic compounds and ionic structures that is limited exteriorly by the mitochondrial outer membrane and interiorly by the mitochondrial inner membrane. Molecular compounds or molecular structures or ionic compounds or ionic structures or molecular compounds and molecular structures and ionic compounds and ionic structures that exist within the IMS are considered part of the IMS.

The term “lactic acid” or “lactate”, as used herein, means the molecular compound CH₃CH(OH)CO₂H with a hydroxyl group adjacent to the carboxyl group constituting the alpha-hydroxy acid form and/or the molecular compound CH₃CH(OH)CO₂H the conjugate base and/or CH₃CH(OH)CO₂ the ionized form and/or “L” and/or “D” stereo isomers.

The term “metformin” or “met” or “3-(diaminomethylidene)-1,1-dimethylguanidine” or “dimethylbiguanidine”, as used herein, means molecular compounds or molecular structures or molecular compounds and molecular structures that are BG agents described by simplified molecular-input line-entry system (SMILES) as; CN(C)C(═N)N═C(N)N and; metabolites and/or prodrugs and/or conjugates and/or other such derivatives and/or analogs and/or related compounds of the molecular structure and/or the molecular compound described by simplified molecular-input line-entry system (SMILES)as; CN(C)C(═N)N═C(N)N.

The term “metabolites” or “metabolite” as used herein, means the intermediate products and/or end products of single enzymatic reactions and/or metabolic pathways encompassing multiple enzymatic reactions. The term metabolite means small molecules and metabolites may possess various functions, including but not limited to bioenergetic substrate and/or structure and/or signaling and/or stimulatory and/or inhibitory effects on enzymes and/or catalytic activity, such as but not limited to acting as cofactor to an enzyme and/or anti-oxidant and/or anti-infective and/or growth and/or development and/or interactions with other biological systems. Additionally, the term “metabolite” includes the autocrine or endocrine or neurological or immunological or genetic structures or actions or structures and actions or the autocrine and endocrine and neurological and immunological and genetic structures or actions or structures and actions which regulate metabolite generation, as well as the organelles or cells or tissues or organs or organ systems or organisms or organelles and cells and tissues and organs and organ systems and organisms and routes of transport (including but not limited to circulatory or lymphatic or circulatory and lymphatic transport) utilized by the biological system in the construction or maintenance or the construction and maintenance of metabolite generation.

The term “mifepristone” or “mife” or “RU486”, as used herein, means the family of molecular compounds or molecular structures or molecular compounds and molecular structures described by simplified molecular-input line-entry system (SMILES) as; O═C5\C═C4/C(═C3/[C@@H](c1ccc(N(C)C)cc1)C[C@]2([C@@H(CC[C@]2(C#CC)O)[C@@H]3CC4)C)CC5 and referred to as RU38486, or RU42633 or RU42698 or 17-(3-hydroxy-11-(3-(4-dimethyl-aminophenyl)-17-a-(1-propynyl)-estra-4,9-dien-3-one) or 11-(3-(4dimethylaminophenyl)-17-(3-hydroxy-17-a-(1-propynyl)-estra-4,9-dien-3-one), or 11(3-[p-(Dimethylamino)phenyl]-17(3-hydroxy-17-(1-propynyl)-estra-4,9-dien-3-one or 11(3-(4-dimethyl-aminophenyl)-17(3-hydroxy-17a-(prop-1-ynyl)-estra-4,9-dien-3-one or 17(3-hydroxy-11(3-(4-dimethylaminophenyl-1)-17a-(propynyl-1)-estra-4,9-diene-3-one or 17(3-hydroxy-11(3-(4-30 dimethylaminophenyl-1)-17a-(propynyl-1)-E or (11(3,17(3)-11-[4-dimethylamino)-phenyl]-17-hydroxy-17-(1-propynyl)estra-4,9-dien-3-one or and 11[3-[4-(N,N-dimethylamino) phenyl]-17a-(prop-1-ynyl)-D-4,9-estradiene-17(3-ol-3-one) or 17beta-Hydroxy-11beta-[4-(methylamino)-phenyl]-17alpha-(1-propinyl)-estra-4,9-dien-3-one or RU38486 and RU42633 and RU42698 and 17-(3-hydroxy-11-(3-(4-dimethyl-aminophenyl)-17-a-(1-propynyl)-estra-4,9-dien-3-one) and 11-(3-(4dimethylaminophenyl)-17-(3-hydroxy-17-a-(1-propynyl)-estra-4,9-dien-3-one) and 11(3-[p-(Dimethylamino)phenyl]-17(3-hydroxy-17-(1-propynyl)-estra-4,9-dien-3-one and 11(3-(4-dimethyl-aminophenyl)-17(3-hydroxy-17a-(prop-1-ynyl)-estra-4,9-dien-3-one and 17(3-hydroxy-11(3-(4-dimethylaminophenyl-1)-17a-(propynyl-1)-estra-4,9-diene-3-one and 17(3-hydroxy-11(3-(4-30 dimethylaminophenyl-1)-17a-(propynyl-1)-E and (11(3,17(3)-11-[4-dimethylamino)-phenyl]-17-hydroxy-17-(1-propynyl)estra-4,9-dien-3-one and 11[3-[4-(N,N-dimethylamino) phenyl]-17a-(prop-1-ynyl)-D-4,9-estradiene-17(3-ol-3-one) and 17beta-Hydroxy-11beta-[4-(methylamino)-phenyl]-17alpha-(1-propinyl)-estra-4,9-dien-3-one or analogs thereof and; metabolites and/or prodrugs and/or conjugates and/or other such derivatives and/or analogs and/or related compounds of the molecular structure and/or the molecular compound described by simplified molecular-input line-entry system (SMILES) as; O═C5\C═C4/C(═C3/[C@@H](c1ccc(N(C)C)cc1)C[C@]2([C@@H(CC[C@]2(C#CC)O)[C@@H]3CC4)C)CC5.

The term “mitochondrial matrix” or “matrix”, as used herein, means a space interior to the inner mitochondrial membrane that may or may not contain molecular compounds or molecular structures or ionic compounds or ionic structures or molecular compounds and molecular structures and ionic compounds and ionic structures and possesses dimensions defined by the mitochondrial inner membrane, including the cristae. Molecular compounds or molecular structures or ionic compounds or ionic structures or molecular compounds and molecular structures and ionic compounds and ionic structures contained in the matrix that do not exist wholly or partially, in the mitochondrial inner membrane are considered part of the matrix.

The term “mitochondrial inner membrane” or “MIM” or “inner mitochondrial membrane” as used herein, means the molecular compounds or molecular structures or ionic compounds or ionic structures or spaces (such as but not limited to; pores or channels or pores and channels) or the temporal-spatial arrangement of these elements or molecular compounds and molecular structures and ionic compounds and ionic structures and spaces and the temporal-spatial arrangement of these elements contained in the structure that is defined exteriorly by the mitochondrial intermembrane space and interiorly by the matrix. Molecular compounds or molecular structures or ionic compounds or ionic structures or spaces or molecular compounds and molecular structures and ionic compounds and ionic structures and spaces which exist or partially exist within the MIM are considered part of the MIM.

The term “mitochondrial outer membrane” or “MOM” or “outer mitochondrial membrane” as used herein, means the molecular compounds or molecular structures or ionic compounds or ionic structures or spaces (such as but not limited to; pores or channels or pores and channels) or the temporal-spatial arrangement of these elements or molecular compounds and molecular structures and ionic compounds and ionic structures and spaces and the temporal-spatial arrangement of these elements contained in the structure that is defined exteriorly by the cytosol when the mitochondrion is present within an intact cell or the external medium when the mitochondrion is isolated and interiorly by the mitochondrial intermembrane space.

The term “mitochondrial reactive oxygen species generation” or “mitochondrial ROS generation” or “a mitochondrial process known to generate reactive oxygen species (ROS)”, as used herein, means structures or actions or structures and actions of the mitochondrial matrix or the mitochondrial inner membrane or the mitochondrial intermembrane space or the mitochondrial outer membrane or the mitochondrial matrix and the mitochondrial inner membrane and the mitochondrial intermembrane space and the mitochondrial outer membrane that gives rise directly or indirectly or directly and indirectly to reactive oxygen species (ROS) or reactive nitrogen species (RNS) or free radicals (FR) or ROS and RNS and FR.

The term “mitochondrial process” or “mitochondrial processes” as used herein, means structures or actions or structures and actions of the mitochondrial matrix or the mitochondrial inner membrane or the mitochondrial intermembrane space or the mitochondrial outer membrane or the mitochondrial matrix and the mitochondrial inner membrane and the mitochondrial intermembrane space and the mitochondrial outer membrane that gives rise directly or indirectly or directly and indirectly to the maintenance of mitochondrial structure and/or function and/or biogenesis and/or involution. Additionally, the term “mitochondrial process” includes the autocrine or endocrine or neurological or immunological or genetic structures or actions or structures and actions or the autocrine and endocrine and neurological and immunological and genetic structures or actions or structures and actions which regulate mitochondrial processes, as well as the organelles or cells or tissues or organs or organ systems or organisms or organelles and cells and tissues and organs and organ systems and organisms and routes of transport (including but not limited to circulatory or lymphatic or circulatory and lymphatic transport) utilized by the biological system in the construction or maintenance or the construction and maintenance of mitochondrial processes.

The term “modify” or “modifies” or “modified” as used herein, means a change from a state of existence pertaining to; structures or actions or structures and actions and where the change may include a physical component or a temporal component or a spatial component or a physical and temporal and spatial component.

The term “molecular compound” or “molecule” as used herein, means a group of two or more atoms bound together via covalent bonds, whereas other binding forces such as but not limited to; ionic bonds or hydrogen bonds or dipole-dipole interactions or ionic bonds and hydrogen bonds and dipole-dipole interactions may be present and may possess a net neutral charge or a net positive charge or a net negative charge.

The term “molecular structure” as used herein, means an entity formed from at least one molecular compound that may or may not possess non-covalent bonds including but not limited to ionic bonds or hydrogen bonds or dipole-dipole interactions or ionic bonds and hydrogen bonds and dipole-dipole interactions and may possess a net neutral charge or a net positive charge or a net negative charge.

The term “nucleoside phosphate molecules” or “nucleoside phosphate”, as used herein, means molecular compounds or molecular structures or molecular compounds and molecular structures consisting of a nitrogenous base and a pentose, such as ribose or deoxyribose, and at least one phosphate group.

The term “metabolic substrate”, as used herein, means molecular compounds or molecular structures or ionic compounds or ionic structures or molecular compounds and molecular structures and ionic compounds and ionic structures that through interaction with a biological system or elements of a biological system or a biological system and elements of a biological system, directly or indirectly or directly and indirectly results in an increased capacity of said biological system or said elements of a biological system or said biological system and said elements of a biological system to perform Work (W).

The term “ionic compound” or “ion” as used herein, means a group of at least one atom where the net electrical charge is positive or negative and interactions with other atoms are primarily characterized by electrostatic forces or ionic bonding or electrostatic forces and ionic bonding.

The term “ionic structure” as used herein, means an entity formed by at least one ionic compound possessing a net neutral charge or net positive charge or negative charge and interactions with other atoms are primarily characterized by electrostatic forces or ionic bonding or electrostatic forces and ionic bonding.

The term “impinge” or “impingement” or “impinging” as used herein, means a direct or indirect or direct and indirect inhibiting effect on structures or actions or structures and actions.

The term “inhibit” or “inhibits” or “inhibiting” as used herein, means a direct or indirect or direct and indirect effect on structures or actions or structures and actions that causes an arrest of occurrence or reduction in magnitude of occurrence or reduction in rate of occurrence or prevention of occurrence or an arrest of occurrence and reduction in magnitude of occurrence and reduction in rate of occurrence and prevention of occurrence.

The term “oxidative phosphorylation” or “OXPHOS”, as used herein, means the component of aerobic metabolism that occurs as a result of interaction between the mitochondrial matrix, the mitochondrial inner membrane, the intermembrane space and the mitochondrial outer membrane that establishes proton concentration in the IMS and where the flow of protons down an electrochemical gradient from the IMS to the matrix through the MIM at ATP synthase drives the phosphorylation of adenosine diphosphate to ATP, ATP-ADP translocase transports ATP out of the matrix and ADP into the matrix. “OXPHOS” also means the autocrine or endocrine or neurological or immunological or genetic structures or actions or structures and actions or autocrine and endocrine and neurological and immunological and genetic structures or actions or structures and actions which regulate the process of “OXPHOS”, as well as the organelles or cells or tissues or organs or organ systems or organisms or organelles and cells and tissues and organs and organ systems and organisms and routes of transport (including but not limited to circulatory or lymphatic or circulatory and lymphatic transport) utilized by the biological system in the performance or maintenance or performance and maintenance of “OXPHOS”.

The term “oxidative stress”, as used herein, means the direct or indirect or direct and indirect effects resulting from elements of a biological system interacting with ROS or RNS or FR or other oxidative agents, including but not limited to photon radiation or ROS and RNS and FR and other oxidative agents, including but not limited to photon radiation and includes but is not limited; to the transformation of molecular compounds or the transformation of molecular structures or the transformation of ionic compounds or the transformation of ionic structures or the alteration of chemical reactions or the alteration of the properties of chemical reactions (including but not limited to reaction rate or quotient or reaction rate and quotient) or to the transformation of molecular compounds and the transformation of molecular structures and the transformation of ionic compounds and the transformation of ionic structures and the alteration of chemical reactions and the alteration of the properties of chemical reactions (including but not limited to reaction rate or quotient or reaction rate and quotient) and the alteration of autocrine or endocrine or neurological or immunological or genetic structures or actions or structures and actions or the alteration of autocrine and endocrine and neurological and immunological and genetic structures or actions or structures and actions.

The term “phenformin” or “1-(diaminomethylidene)-2-(2phenylethyl)guanidine”, as used herein, means molecular compounds or molecular structures or molecular compounds and molecular structures that are BG agents described by simplified molecular-input line-entry system (SMILES)as; C1=CC═C(C═C1)CCN═C(N)N═C(N)N and; metabolites and/or prodrugs and/or conjugates and/or other such derivatives and/or analogs and/or related compounds of the molecular structure and/or the molecular compound described by simplified molecular-input line-entry system (SMILES) as; C1=CC═C(C═C1)CCN═C(N)N═C(N)N and includes 4-hydroxyphenformin, described by simplified molecular-input line-entry system (SMILES)as; C1=CC(═CC═C1CCN═C(N)N═C(N)N)O and; metabolites and/or prodrugs and/or conjugates and/or other such derivatives and/or analogs and/or related compounds of the molecular structure and/or the molecular compound described by simplified molecular-input line-entry system (SMILES) as; C1=CC(═CC═C1CCN═C(N)N═C(N)N)O.

The term “prodrug” or “prodrugs”, as used herein, means a molecular compound and/or molecular structure and/or ionic compound and/or ionic structure that, after administration to a biological system is metabolized into a pharmacologically active composition. Prodrugs are pharmacologically inactive compositions that are metabolized into an active form within a biological system. Instead of administering a pharmaceutical composition directly to a biological system, a prodrug may be administered to a biological system in order to improve how a pharmaceutical composition is absorbed and/or distributed and/or metabolized and/or excreted. Prodrugs may be engineered and/or designed to improve bioavailability when the active pharmaceutical composition is itself poorly absorbed and/or poorly distributed and/or poorly metabolized and/or poorly excreted. A prodrug may be used to improve how selectively a pharmaceutical composition interacts with biological systems and/or processes and/or structures that are not the intended target of the pharmaceutical composition.

The term “pyruvate” or “pyruvic acid”, as used herein, means the molecular compound (CH₃COCOOH) with a carboxylic acid and a ketone functional group and/or the conjugate base CH₃COCOO⁻. Pyruvate is a key intermediate in several metabolic pathways. Pyruvic acid can be synthesized from glucose catabolism via glycolysis, and may be converted back to glucose via gluconeogenesis acid alanine and can be converted into ethanol or lactic acid via fermentation. Pyruvic acid supplies free energy to eukaryotic biological systems through the citric acid cycle (also known as the Krebs cycle) during aerobic respiration and alternatively ferments to produce lactate during anaerobic respiration.

The term “reactive oxygen species” or “ROS”, as used herein, means molecular compounds or ionic compounds or molecular structures or ionic structures or molecular compounds and ionic compounds and molecular structures and ionic structures characterized by the inclusion of a partially reduced oxygen atom or an oxygen atom susceptible to partial reduction or a partially reduced oxygen atom and an oxygen atom susceptible to partial reduction including but not limited to; singlet oxygen or superoxide or hydroperoxyl or peroxide or hydroxyl radical or hypochlorous acid or peroxynitrite or nitrogen dioxide or nitrosoperoxycarbonate or dinitrogen trioxide or singlet oxygen and superoxide and hydroperoxyl and peroxide and hydroxyl radical and hypochlorous acid and peroxynitrite and nitrogen dioxide and nitrosoperoxycarbonate and dinitrogen trioxide.

The term “reactive nitrogen species” or “RNS”, as used herein, means molecular compounds or ionic compounds or molecular structures or ionic structures or molecular compounds and ionic compounds and molecular structures and ionic structures which are partially reduced or susceptible to partial reduction or partially reduced and susceptible to partial reduction and possess a nitrogen atom and include but are not limited to; peroxynitrite, nitrogen dioxide, nitrosoperoxycarbonate, dinitrogen trioxide.

The term “enzyme”, as used herein, means molecular compounds or molecular structures or molecular compounds and molecular structures that directly or indirectly or directly and indirectly lower the activation energy of a specific chemical reaction or multiple chemical reactions or a specific chemical reaction and multiple chemical reactions and molecular compounds or molecular structures or ionic compounds or ionic structures or molecular compounds and molecular structures and ionic compounds and ionic structures utilized in the structures or actions or structures and actions of the enzyme and the autocrine or endocrine or neurological or immunological or genetic structures or actions or structures and actions or the autocrine and endocrine and neurological and immunological and genetic structures or actions or structures and actions utilized in the construction or maintenance or construction and maintenance of enzymes.

The term “Xanthine oxidase” or “Xanthine: NAD+ oxidoreductase” or “Xanthine dehydrogenase”, as used herein, means an enzyme possessing the properties necessary to catalyze a chemical process defined by Enzyme Commission number EC 1.17.1.4 or Enzyme Commission number EC 1.17.3.2 or Enzyme Commission number EC 1.17.1.4 and EC 1.17.3.2 and includes the autocrine or endocrine or neurological or immunological or genetic structures or actions or structures and actions or autocrine and endocrine and neurological and immunological and genetic structures or actions or structures and actions which regulate “xanthine oxidase”, as well as the organelles or cells or tissues or organs or organ systems or organisms or organelles and cells and tissues and organs and organ systems and organisms and routes of transport (including but not limited to circulatory or lymphatic or circulatory and lymphatic transport) utilized by the biological system in the construction or maintenance or construction and maintenance of “xanthine oxidase”.

The term “pharmaceutically acceptable salts”, as used herein, means salts of the active principal agents which are prepared with acids or bases that are tolerated by a biological system or tolerated by a subject or tolerated by a biological system and tolerated by a subject when administered in a therapeutically effective amount. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include, but are not limited to; sodium, potassium, calcium, ammonium, organic amino, magnesium salt, lithium salt, strontium salt or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include, but are not limited to; those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like.

Certain specific compounds of the present invention contain both basic and acidic functionalities that allow the compounds to be converted into either base or acid addition salts. Neutral forms of active principals may be regenerated by contacting the salt with a base or acid and isolating the parent compound in the conventional manner. The parent form of the compound differs from the various salt forms in certain physical properties, such as solubility in polar solvents.

In addition to salt forms, the present invention provides compounds, which are in a prodrug form. “Prodrug”, as used herein, means those compounds that readily undergo chemical changes under physiological conditions to provide active principal agents of the present invention. Additionally, prodrugs can be converted to the active principal agents of the present invention by chemical or biochemical or chemical and biochemical methods in an ex vivo environment. For example, prodrugs can be slowly converted to active principal agents of the present invention when placed in a transdermal patch reservoir with a suitable enzyme or chemical reagent or enzyme and chemical reagent.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, tautomers, geometric isomers and individual isomers are encompassed within the scope of the present invention.

The compounds of the present invention may also contain unnatural proportions of atomic isotopes at one or more of the atoms that constitute such compounds. For example, the compounds may be radiolabeled with radioactive isotopes, such as, tritium (³H), iodine-125 (¹²⁵I) or carbon-14 (¹⁴C). All isotopic variations of the compounds of the present invention, whether radioactive or not, are encompassed within the scope of the present invention.

When referring to an active principal agent, embodiments of the present invention encompass not only the specified molecular entity but also its pharmaceutically acceptable or pharmacologically active or pharmaceutically acceptable and pharmacologically active analogs, including, but not limited to, salts, esters, amides, prodrugs, conjugates, active metabolites, and other such derivatives, analogs, and related compounds.

The terms “treat”, “treating” and “treatment”, as used herein, means a reduction in; severity or frequency or magnitude or severity and frequency and magnitude of diseases or disorder or conditions or diseases and disorders and conditions, or improvement of damage to a biological system or remediation of damage to a biological system or improvement and remediation of damage to a biological system. In certain aspects, the terms “treat”, “treating” and “treatment”, as used herein, refer to the prevention of the occurrence of diseases or disorders or conditions or diseases and disorders and conditions.

The term “dosage form” or “unit dosage form” denotes any form of a pharmaceutical composition that contains an amount of active agent sufficient to achieve a measurable effect or concentration in the blood stream with a single administration. When the formulation is a tablet or capsule, the dosage form is usually one such tablet or capsule. The frequency of administration that will provide the most effective results in an efficient manner without overdosing will vary with the characteristics of the particular active agent, including both its pharmacological characteristics and its physical characteristics, such as hydrophilicity.

The term “controlled release” refers to a drug-containing formulation or fraction or component thereof (e.g. one of more of several active ingredients) in which release of the drug or component intended for non-immediate release is not immediate, i.e., with a “controlled release” formulation, administration does not result in immediate disintegration and dissolution of the controlled drug upon. The term is used interchangeably with “nonimmediate release” as defined in Remington: The Science and Practice of Pharmacy, Nineteenth Ed. (Easton, Pa.: Mack Publishing Company, 1995). In general, the term “controlled release” as used herein includes sustained-release, modified release and delayed release formulations.

The term “sustained release” (synonymous with “extended release”) is used in its conventional sense to refer to a drug formulation that provides for gradual release of a drug over an extended period of time, and that preferably, although not necessarily, results in substantially constant blood levels of a drug over an extended time period. The term “delayed release” is also used in its conventional sense, to refer to a drug formulation which, following administration to a patient provides a measurable time delay before drug is released from the formulation into the patient's body.

By “pharmaceutically acceptable” is meant a material that is not biologically or otherwise undesirable, i.e., the material may be incorporated into a pharmaceutical composition administered to a patient without causing any undesirable biological effects or interacting in a deleterious manner with any of the other components of the composition in which it is contained. When the term “pharmaceutically acceptable” is used to refer to a pharmaceutical carrier or excipient, it is implied that the carrier or excipient has met the required standards of toxicological and manufacturing testing or that it is included on the Inactive Ingredient Guide prepared by the U.S. Food and Drug administration. “Pharmacologically active” (or simply “active”) as in a “pharmacologically active” derivative or analog, refers to a derivative or analog having the same type of pharmacological activity as the parent compound and approximately equivalent in degree. As used herein, “subject” or “individual” or “patient” refers to any subject for whom or which therapy is desired, and generally refers to the recipient of the therapy to be practiced according to the invention. The subject can be any vertebrate, but will typically be a mammal. If a mammal, the subject will in many embodiments be a human, but may also be a domestic livestock, laboratory subject or companion animal.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits and or ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

The publications discussed herein are provided solely for their disclosure prior to the filing date of the present application. Nothing herein is to be construed as an admission that the present invention is not entitled to antedate such publication by virtue of prior invention. Further, the dates of publication provided maybe different from the actual publication dates that may need to be independently confirmed.

### DETAILED DESCRIPTION OF THE INVENTION

The embodiments of the present invention are directed to pharmaceutical compositions for the treatment or prevention or the treatment and prevention of diseases or disorders or conditions or diseases and disorders and conditions associated with bioenergetic dysfunction. In particular, the present invention provides compositions and methods of treatment and/or prevention that alter bioenergetic processes in a eukaryotic biological system by causing an inhibition of anaerobic metabolic processes when a therapeutically effective amount of a pharmaceutical composition that is an embodiment of the present invention is administered to a eukaryotic biological system possessing mitochondria.

The term “therapeutically effective amount”, as used herein, means the quantity of active principal agents or compositions containing active principal agents or the quantity of active principal agents and compositions containing active principal agents, that constitute effective treatment, as determined via art-recognized methods and selected by those skilled in the art, upon administration to a subject and/or patient.

The term “administering to” or “administered to”, as used herein, means the process of introducing an embodiment of the invention into a eukaryotic biological system or a subject's body or a patient's body or a eukaryotic biological system and a subject's body and a patient's body via a term of art-recognized means of introduction.

Namely, a pharmaceutical composition can be formulated that contains at least two active principal agents where; at least one of the active principals is an agent that reduces the activity of a mitochondrial ROS generating process and; at least one other active principal agent contributes a reduced rate of mitochondrial oxygen consumption and; the pharmaceutical compositions inhibit anaerobic metabolic activity in a eukaryotic biological system.

An active principal agent may demonstrate the ability to inhibit a mitochondrial process that generates ROS and contribute to a reduced rate of mitochondrial oxygen consumption.

“Contributes to a reduced rate of mitochondrial oxygen consumption”, as used herein, is a term of art meaning; an active principal that “contributes to a reduced rate of mitochondrial oxygen consumption” results in a decreased rate of oxygen consumption at the level of the mitochondrion or cell or tissue or organ or organ system or organism or mitochondrion and cell and tissue and organ and organ system and organism compared to the rate of oxygen consumption present in untreated basal metabolic conditions and/or non-treated maximal metabolic conditions (such as metabolic conditions that have been perturbed by an ionophore mitochondrial uncoupler, including but not limited to carbonyl cyanide-p-trifluoromethoxyphenylhydrazone (FCCP)) in eukaryotic biological systems possessing mitochondria.

Examples of mitochondrial processes that generate ROS include but are not limited to; the enzymatic action of NADH: ubiquinone reductase and/or the enzymatic action of Succinate dehydrogenase and/or the enzymatic action of Ubiquinol cytochrome-c reductase and/or the enzymatic action of Xanthine oxidase and/or the interaction of anthracyclines, such as doxorubicin, with Ubiquinol cytochrome-c reductase.

Embodiments of the present invention in the form of pharmaceutical compositions possessing at least two active principal agents where; at least one of the active principal agents is an inhibitor of a mitochondrial process known to generate ROS and; at least one of the other active principals contributes to a reduced rate of mitochondrial oxygen consumption possess unexpected emergent properties, namely, said pharmaceutical compositions demonstrate the ability to inhibit on anaerobic metabolic processes in a eukaryotic biological system, causing a lower than expected level of anaerobic metabolic activity.

The inhibition of anaerobic metabolic processes resulting from administration, to a subject, of a therapeutically effective amount of an embodiment of the present invention can be applied as methods of treatment or methods of prevention or methods of prevention and treatment for diseases or disorders or conditions or diseases and disorders and conditions associated with bioenergetic dysfunction and/or oxidative stress.

The application of embodiments of the present invention as methods of treatment and/or prevention to diseases and/or disorders and/or conditions associated with bioenergetic dysfunction is consistent with fundamental concepts of the prior art pertaining to eukaryotic bioenergetic metabolism, such as, but not limited to; anaerobic threshold or oxygen debt or anaerobic threshold and oxygen debt.

Fundamental concepts of the prior art pertaining to eukaryotic bioenergetic metabolism, such as, but not limited to; anaerobic threshold or oxygen debt or anaerobic threshold and oxygen debt, instruct that the primary source of free energy production, in the form of nucleoside triphosphate molecules, in a eukaryotic biological system possessing mitochondria is aerobic metabolism.

Fundamental concepts of the prior art pertaining to eukaryotic bioenergetic metabolism instruct that under conditions of significantly increased demand for free energy, such as but not limited to, intense physical exercise, an increased level of anaerobic metabolic activity can be expected as the eukaryotic biological system attempts of satisfy the increased free energy demand. (Baker J. Maximal shuttle running over 40 m as a measure of anaerobic performance. Br J Sp Med 1993; 27(4): 228-232).

Results of Experiment 2 provide confirmatory evidence in support of the fundamental concepts of the prior art pertaining to bioenergetic metabolism in a eukaryotic biological system, namely, that the performance of intense physical activity by a human subject, results in an increased level of anaerobic metabolic activity as indicated by increased blood lactate concentrations observed; immediately following a bout of intense physical exercise, 3 minutes after a bout of intense physical exercise and 5 minutes after a bout of intense physical exericse, relative to basal blood lactate concentrations observed at rest prior to undertaking a bout of intense physical exercise (Table 28D, FIG. 4a). FIG. 4a shows significantly increased blood lactate levels in a human subject post intense physical exercise, relative to basal blood lactate levels in a human subject at rest prior to undertaking a bout of intense physical exercise.

Fundamental concepts of the prior art pertaining to eukaryotic bioenergetic metabolism instruct that under conditions of decreased capacity of a eukaryotic biological system to conduct aerobic metabolic activity, such as but not limited to, hypoxic conditions, an increased level of anaerobic metabolic activity can be expected as the eukaryotic biological system attempts of satisfy free energy demands. (Kottmann R. et al. Lactic Acid Is Elevated in Idiopathic Pulmonary Fibrosis and Induced Myofibroblast Differentiation via pH-Dependent Activation of Transforming Growth Factor-B. Am J Respir Crit Care Med. 2012 Oct. 15; 186(8): 740-751).

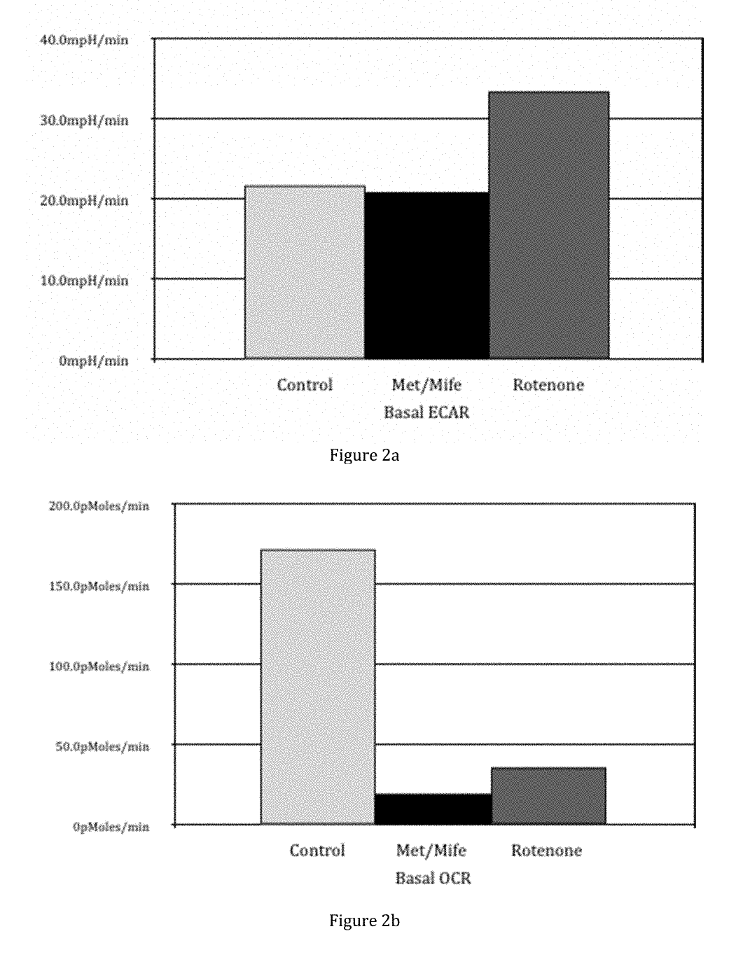

Results of Experiment 1 provide confirmatory evidence in support of the fundamental concepts of the prior art pertaining to eukaryotic bioenergetic metabolism, namely that under conditions of decreased capacity of a eukaryotic biological system to conduct aerobic metabolic activity, an increased level of anaerobic metabolic activity can be expected. C2C12 murine myoblasts, treated with the known mitochondrial inhibiting agent Rotenone, demonstrated significantly impaired mitochondrial aerobic metabolism, as indicated by a decreased oxygen consumption rate (OCR), (FIG. 2b, Table 27A5, Table 27B5, Table 27C5).

Additionally, C2C12 murine myoblasts treated with Rotenone, and demonstrating impaired levels of mitochondrial aerobic metabolism, demonstrated a corresponding increased level of anaerobic metabolic activity, as indicated by an increased extracellular acidification rate (ECAR), in Rotenone treated C2C12 murine myoblasts, as the eukaryotic biological system attempted to satisfy free energy demands. (FIG. 2a, Table 27A2, Table 27B2, Table 27C2). FIG. 2a shows the results of extracellular flux analysis comparing the elevated level of anaerobic metabolic activity of myoblasts treated with Rotenone to the lower level of anaerobic metabolic activity observed for control condition C2C12 murine myoblasts, which experienced no inhibition of aerobic metabolic activity.

Despite the well established fundamental concepts of the prior art pertaining to eukaryotic bioenergetic metabolism, instances of diseases and/or disorders and/or conditions associated with bioenergetic dysfunction and/or oxidative stress exist in which affected eukaryotic biological systems adopt an increased reliance on anaerobic metabolic activity, despite adequate availability of oxygen and metabolic substrate.

An example of the phenomenon of diseases and/or disorders and/or conditions associated with bioenergetic dysfunction and/or oxidative stress resulting in the development of a greater reliance on free energy generation by anaerobic metabolic processes is the pathological bioenergetic metabolism demonstrated by cancer cells exhibiting the “Warburg Effect”, where an increased relative level of anaerobic metabolic activity is observed despite the presence of adequate concentrations of oxygen and metabolic substrate (Jiansheng X. et al. Beyond Warburg Effect-dual metabolic nature of cancer cells. Scientific Reports, 13 May 2014, 4, 4927).

Yet other examples of the phenomenon of diseases and/or disorders and/or conditions associated with bioenergetic dysfunction and/or oxidative stress resulting in the development of a greater reliance on free energy generation by anaerobic metabolic processes despite the presence of adequate oxygen and metabolic substrate in eukaryotic biological systems include but are not limited to: insulin resistance, sedentary behavior, immobility, microgravity exposure, aging, type 2 diabetes mellitus, diabetic cardiomyopathy and hepatitis (Haffar T, et. al, Impaired fatty acid oxidation as a cause for lipotoxicity in cardiomyocytes. Biochem Biohys Res Commun. 2015 Dec. 4-11; 468(1-2):73-8), (Tucker M, et. al, Impaired fatty acid oxidation in muscle of aging rats perfused under basal conditions. Am J Physiol Endocrinol Metab. 2002 May; 282(5):E1102-9), (Sato C, et. al. Impaired mitochondrial β-oxidation in patients with chronic hepatitis C: relation with viral load and insulin resistance, BMC Gastroenterol. 2013; 13: 112), (Du F, et. al, Morphology and Molecular Mechanisms of Hepatic Injury in Rats under Simulated Weightlessness and the Protective Effects of Resistance Training. PLoS One. 2015 May 22; 10(5):e0127047), (DeNies M, et. al, Diet-induced obesity alters skeletal muscle fiber types of male but not female mice. Physiol Rep. 2014 Jan. 1; 2(1): e00204).

Embodiments of the present invention in the form of pharmaceutical compositions possessing at least two active principal agents where; at least one of the active principal agents is an inhibitor of a mitochondrial process known to generate ROS and; at least one of the other active principals contributes to a reduced rate of mitochondrial oxygen consumption and said pharmaceutical compositions demonstrate the ability to inhibit anaerobic metabolic processes in a eukaryotic biological system are applicable as methods of treatment and/or prevention in diseases and/or disorders and/or conditions associated with bioenergetic dysfunction and/or oxidative stress and which demonstrate an increased reliance on anaerobic metabolic processes despite the presence of adequate oxygen and/or metabolic substrate.

Results of Experiment 1 revealed that an exemplary embodiment of the present invention, namely a pharmaceutical composition of [metformin/mifepristone], demonstrated the characteristic trait of inhibiting anaerobic metabolic activity, as indicated by inducing a lower than expected extracellular acidification rate (ECAR), in C2C12 murine myoblasts treated with the pharmaceutical composition [metformin/mifepristone] relative to the level fo anaerobic metabolic activity demonstrated by untreated control condition C2C12 murine myoblasts (FIG. 2a, Table 27A3).

The level of anaerobic metabolic activity, as indicated by ECAR, observed during treatment of C2C12 murine myoblasts with the pharmaceutical composition [metformin/mifepristone] was lower, to a statistically significant degree, than the level of anaerobic metabolic activity, as indicated by ECAR, of C2C12 murine myoblasts treated with Rotenone (FIG. 2a, Table 27A3).

The ability of an exemplary embodiment of the present invention, namely a pharmaceutical composition of [metformin/mifepristone], to elicit a lower level of anaerobic metabolic activity, as indicated by ECAR, relative to the level of anaerobic metabolic activity for C2C12 murine myoblasts treated with Rotenone, was not due to the pharmaceutical composition [metformin/mifepristone] failing to inhibit mitochondrial aerobic metabolism to the same degree as Rotenone, as [metformin/mifepristone] inhibited mitochondrial oxygen uptake to a greater degree than Rotenone.

Experiment 1 results revealed that treatment with an exemplary embodiment of the present invention, namely the pharmaceutical composition [metformin/mifepristone], demonstrated a significantly greater inhibition of mitochondrial aerobic metabolism, as indicated by a lower oxygen consumption rate (OCR), in C2C12 murine myoblasts treated with [metformin/mifepristone], relative to the inhibition of mitochondrial aerobic metabolism, as indicated OCR, resulting from the treatment of C2C12 murine myoblasts with the known mitochondrial inhibitor Rotenone (FIG. 2b, Table 27A6, Table 27B6, Table 27C6). FIG. 2b shows significantly reduced mitochondrial oxygen consumption rate of myoblasts treated with Rotenone compare to control.

Further, an exemplary embodiment of the present invention, consisting of the pharmaceutical composition [metformin/mifepristone], demonstrated the ability to elicit a level of anaerobic metabolic activity, as indicated by ECAR in C2C12 murine myoblasts, that was lower to a statistically significant degree relative to the level of anaerobic metabolic activity demonstrated by non-treated Control condition C2C12 murine myoblasts under maximal metabolic conditions (FCCP perturbed metabolism) (Table 27A7, Table 27B8).

The ability of an exemplary embodiment of the present invention, namely the pharmaceutical composition [metformin/mifepristone], to elicit a disproportionately lower level of anaerobic metabolic activity, as indicated by the ECAR, relative to the anaerobic metabolic activity of Control condition C2C12 murine myoblasts was not due to the pharmaceutical composition [metformin/mifepristone] failing to inhibit mitochondrial metabolism to the same degree as Control C2C12 murine myoblasts.

Experiment 1 results revealed that an exemplary embodiment of the present invention, namely the pharmaceutical composition [metformin/mifepristone], demonstrated significantly greater inhibition of aerobic metabolic activity, as indicated by the significantly lower OCR values of C2C12 murine myoblasts treated with [metformin/mifepristone] relative to the OCR of Control condition C2C12 murine myoblasts (FIG. 2b, Table 27A4, Table 27A8, Table 27B4, Table 27B9, Table 27C4, Table 27C9).

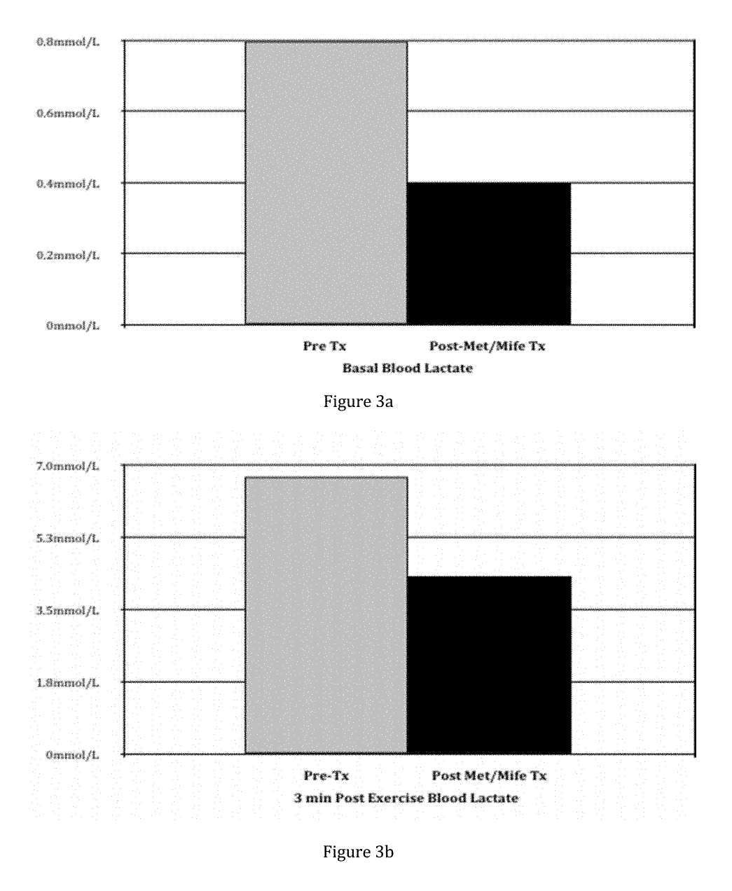

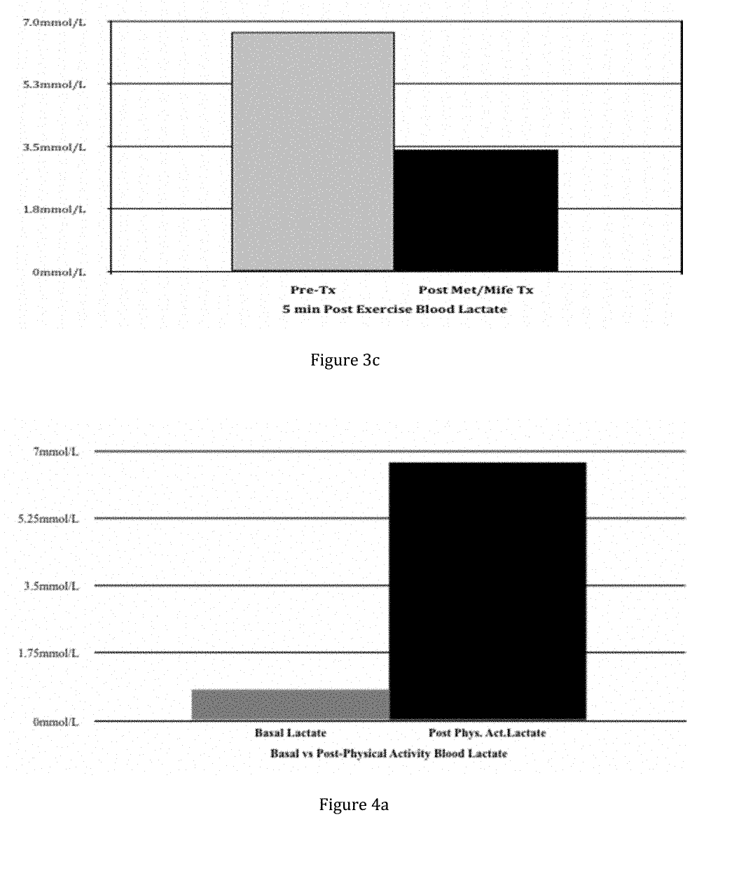

In vivo experimental observations of Experiment 2 confirm the in-vitro observations of Experiment 1. Experiment 2 results revealed that an exemplary embodiment of the present invention consisting of the pharmaceutical composition [metformin/mifepristone], demonstrated the characteristic of eliciting an unexpectedly lower level of anaerobic metabolic activity, when administered to a human subject. The observation of decreased levels of basal blood lactate in a human subject treated with [metformin/mifepristone], compared to the level of basal blood lactate in an untreated human subject demonstrates the intrinsic nature of the ability to inhibit anaerobic metabolic activity that is characteristic of embodiments of the present invention. (FIG. 3a, FIG. 3b, FIG. 3c).

FIG. 3a demonstrates that basal blood lactate levels, an indicator of anaerobic metabolic activity, were observed to be significantly greater in an untreated human subject compared to the significantly lower basal blood lactate levels observed in a human subject following treatment with the pharmaceutical composition [metformin/mifepristone]. FIG. 3b demonstrates that blood lactate levels recorded 3 minutes after an intense bout of physical activity, in an untreated human subject, were observed to be significantly greater than the blood lactate levels recorded 3 minutes after an intense bout of physical activity, in a human subject following treatment with the pharmaceutical composition [metformin/mifepristone]. FIG. 3c demonstrates that blood lactate levels recorded 5 minutes after an intense bout of physical activity, in an untreated human subject, were observed to be significantly greater than the blood lactate levels recorded 5 minutes after an intense bout of physical activity, in a human subject following treatment with the pharmaceutical composition [metformin/mifepristone].

Basal blood lactate levels, in a human subject, following treatment with an exemplary embodiment of the present invention, namely the pharmaceutical composition [metformin/mifepristone], were a *minimum of 50% less than the basal blood lactate levels observed in an untreated human subject. *Treatment of a human subject with the pharmaceutical composition [metformin/mifepristone] resulted in basal blood lactate levels that registered below the detectable limit of 0.5 mmol/L for the Lactate Scout Plus blood lactate monitor, manufactured by EKF Diagnostics and therefore a value of 0.49 mmol/L was utilized in order to calculate a minimum magnitude of decreased blood lactate values relative to untreated conditions. (Table 28D, Table 28J).

Blood lactate levels, in a human subject, following treatment with an exemplary embodiment of the present invention, namely the pharmaceutical composition [metformin/mifepristone] resulted in blood lactate levels recorded 3 minutes following an intense bout of physical exercise that where 35.8% lower relative to blood lactate levels observed in an untreated human subject that were recorded 3 minutes following an intense bout of physical exercise (Table 28D, Table 28J).

Blood lactate levels, in a human subject, following treatment with the pharmaceutical composition [metformin/mifepristone], resulted in blood lactate levels recorded 5 minutes following an intense bout of physical exercise that where 49.3% lower relative to blood lactate levels observed in an untreated human subject that were recorded 5 minutes following an intense bout of physical exercise (Table 28D, Table 28J).

Experiment 2 results revealed that an exemplary embodiment of the present invention consisting of the pharmaceutical composition of [metformin/mifepristone], when administered to a human subject, demonstrated the characteristic of inhibiting anaerobic metabolic activity and eliciting an increased reliance on aerobic metabolic processes.

Experiment 2 results revealed that Total work (Wₜₒₜₐₗ) performed during an exercise to exhaustion stress test, by a human subject, demonstrated a 13.7% increase following treatment with an exemplary embodiment of the present invention, the pharmaceutical composition [metformin/mifepristone], relative to Wₜₒₜₐₗ performed during an exercise to exhaustion stress test by an untreated human subject. (Table 28D, Table 28J)

Experiment 2 results revealed that the work (W) performed at the 4.6 kg resistance level, the second lowest resistance level, during an exercise to exhaustion stress test, by a human subject, demonstrated a 79.3% increase after treatment with an exemplary embodiment of the present invention consisting of the pharmaceutical composition, [metformin/mifepristone], relative to the W performed at the 4.6 kg resistance level during an exercise to exhaustion stress test, by an untreated human subject. (Table 28D, Table 28J)

Experiment 2 results revealed that the work (W) performed at the 3.2 kg resistance level, the lowest resistance level, during an exercise to exhaustion stress test, by a human subject, demonstrated a 100% increase after treatment with an exemplary embodiment of the present invention consisting of the pharmaceutical composition, [metformin/mifepristone], relative to the W performed at the 3.2 kg resistance level during an exercise to exhaustion stress test, by an untreated human subject. (Table 28D, Table 28J)

While W performed at the heaviest resistance level of 24 kg, during an exercise to exhaustion stress test, by a human subject, demonstrated a 13.8% decrease following treatment with an exemplary embodiment of the present invention consisting of the pharmaceutical composition, [metformin/mifepristone], relative to the W performed at the 24 kg resistance level during an exercise to exhaustion stress test, by an untreated human subject. (Table 28D, Table 28J)

Heavy mechanical loads require greater force to displace them. Human skeletal muscle fibers feature force production characteristics that are inversely proportional to their capacity to generate free energy via aerobic metabolic processes, namely, skeletal muscle fibers with the greatest force production characteristics possess the lowest capacity to generate free energy via aerobic metabolic processes and are thus more reliant on anaerobic metabolic processes. While skeletal muscle fibers with the lowest force production characteristics possess the highest capacity to generate free energy via aerobic metabolic pathways.

The observed effect, that treatment of a human subject with an exemplary embodiment of the present invention, namely the pharmaceutical composition [metformin/mifepristone], resulted in lower blood lactate levels under basal conditions, at 3 minutes post an intense bout of physical activity and at 5 minutes post an intense bout of physical activity compared to blood lactate levels observed in an untreated human subject is demonstrative of the ability of embodiments of the present invention to inhibit anaerobic metabolic activity in a human subject.

The observed effect that treatment of a human subject with an exemplary embodiment of the present invention in the form of the pharmaceutical composition [metformin/mifepristone] decreased W performed at the heaviest resistance load of 24 kg, during an exercise to exhaustion stress test compared to the W performed at the 24 kg resistance load, during an exercise to exhaustion stress test conducted by an untreated human subject is indicative that treatment with the pharmaceutical composition [metformin/mifepristone] decreased anaerobic metabolic activity in a human subject.

The observed effect that treatment of a human subject with the pharmaceutical composition [metformin/mifepristone] resulted in decreased blood lactate levels, increased total W, increased W at the lightest resistance loads of 3.2 kg and 4.6 kg are indicative of decreased anaerobic metabolic activity and increased aerobic metabolic activity relative to the level of anaerobic and aerobic metabolic activity observed in an untreated human subject.

Fundamental principles of the prior art pertaining to pharmacology and physiology hold that a primary effect of the treatment of a eukaryotic biological system with a pharmaceutical agent that is capable of inhibiting a mitochondrial process known to generate ROS, such as the biguanide agents metformin and/or phenformin and/or buformin is an increased level of anaerobic metabolic activity, characterized by increased blood lactate levels.

In fact, lactic acidosis, a condition characterized by excessive blood levels of lactic acid and disruption of homeostatic measures is the most recognized iatrogenic sequelae resulting from treatment with agents such as but not limited to metformin, phenformin and buformin. In fact, concerns over the risk of lactic acidosis resulting from treatment with such agents caused regulatory agencies to remove phenformin and buformin as therapeutic agents from most global markets.

Even when a pharmaceutical agent capable of inhibiting a mitochondrial process known to produce ROS, such as metformin, is not considered unsafe for use as a therapeutic agent, pharmacodynamic and/or pharmacokinetic traits can exist which limit the therapeutic utility of the agent.

An example includes the altered distribution of the pharmaceutical agent metformin in NASH. The prior art has established that NASH alters renal mRNA expression of Oct2 and Mate 1 resulting in impaired kidney transporter expression which alters the pharmacokinetics of metformin by increasing metformin plasma concentration, increasing metformin plasma half-life, increasing metformin mean residence time and decreasing oral clearance rate of metformin. (Clarke J D, et al. Mechanism of Altered Metformin Distribution in Nonalcoholic Steatohpatitis. Diabetes. 2015 September; 64(9):3305-13)

NASH induced alterations in metformin pharmacokinetics and pharmacodynamics results in higher plasma concentrations of metformin and decreased clearance rates of metformin that increase the risk of lactic acidosis and decrease the utility of metformin treatment in the context of NASH.

Thus, the observation that treatment of a human subject with exemplary embodiments of the present invention, such as the pharmaceutical composition [metformin/mifepristone] that does not result in an increased level of anaerobic metabolic activity and the associated increase of blood lactic acid concentrations expected of metformin treatment, but rather causes a reduced level of anaerobic metabolic activity and decreased lactic acid concentrations relative to untreated human control subjects is novel and unexpected in the context of the prior art.

Additionally, the findings that embodiments of the present invention decreased anaerobic metabolic processes and decreased lactic acid concentrations in a human subject constitute emergent properties that allow for the use of pharmaceutical agents such as but not limited to metformin, in the context of embodiments of the present invention to treat diseases and/or disorders and/or conditions such as but not limited to NASH, where the use of a pharmaceutical agent such as but not limited to metformin as a monotherapeutic agent is deemed deleterious or unsafe.

The findings that embodiments of the present invention that inhibit anaerobic metabolic processes and decrease lactic acid concentrations in a human subject constitute emergent properties that allow for the use of pharmaceutical agents such as but not limited to phenformin and buformin in the context of embodiments of the present invention to treat diseases and/or disorders and/or conditions associated with bioenergetic dysfunction and/or oxidative stress, where the use of a pharmaceutical agent such as but not limited to phenformin and buformin as a monotherapeutic agent had been deemed unsafe.

Embodiments of the present invention that inhibit anaerobic metabolic processes and decrease lactic acid concentrations in a human subject constitute an advancement in the art, as previous attempts to engineer therapeutic interventions that inhibit anaerobic metabolic processes, such as the competitive inhibitor of glycolysis 2-Deoxy-d-Glucose which demonstrate cardiotoxicity (Terse P S et al. 2-Deoxy-d-Glucose (2-DG)-Induced Cardiac Toxicity in Rat: NT-proBNP and BNP as Potential Early Cardiac Safety Biomarkers. Int J Toxicol 2016 May; 35(3):284-93).

The prior art has identified the triglyceride: HDL-cholesterol ratio as an indicator or predictor or an indicator and predictor of oxidative stress associated diseases, disorders and conditions such as, but not limited to; coronary disease, insulin resistance, cardio metabolic syndrome, non-alcoholic fatty liver disease (NAFLD), non-alcoholic steatohepatitis (NASH) (Protasio L. et. al High Ratio of Triglycerides to HDL-Cholesterol Predicts Extensive Coronary Disease; Clinics. 2008 August; 63(4):427-432), (Jianfeng L. et. al. Triglycerides and high-density lipoprotein cholesterol ratio compared with homeostasis model assessment insulin resistance indexes in screening for metabolic syndrome in the Chinese obese children: a cross section study; BMC Pediatr. 2015; 15:138), (Fargion S. et. al. Nonalcoholic fatty liver disease and vascular disease: State-of-the-art; World J Gastroenterol. 2014 Oct. 7; 20(37): 13306-13324.)

Results of Experiment 2 revealed that an exemplary embodiment of the present invention, the pharmaceutical composition [metformin/mifepristone], when administered to a human subject, demonstrated the effect of reducing the serum triglyceride: HDL-cholesterol ratio by 29.3% compared to the serum triglyceride: HDL-cholesterol ratio observed in an untreated human subject (FIG. 5a). FIG. 5a demonstrates a significantly decreased triglyceride: HDL cholesterol ratio in a human subject following treatment with the pharmaceutical compound [metformin/mifepristone] compared to the baseline triglyceride: HDL-cholestrol ratio established for the human subject prior to treatment.

The prior art has identified serum C-reactive protein (CRP) as an indicator or predictor or an indicator and predictor of bioenergetic dysfunction and/or oxidative stress associated diseases and/or disorders and/or conditions such as but not limited to; cardio renal metabolic syndrome, cancer, Alzheimer disease, (Pravenec M. et al. Effects of Human C-reactive Protein on Pathogenesis of Features of the Metabolic Syndrome; Hypertension. 2011 April; 57(4)), (Jian-Hua Y. et al. C-Reactive Protein as a Prognostic Factor for Human Osteosarcoma: A Meta-Analysis and Literature Review; PLoS One. 2014; 9(5): e94632), (O'Bryant S. et al. Decreased C-Reactive Protein Levels in Alzheimer Disease; J Geriatr Psychiatry Neurol. 2010 March; 23(1):49-53)

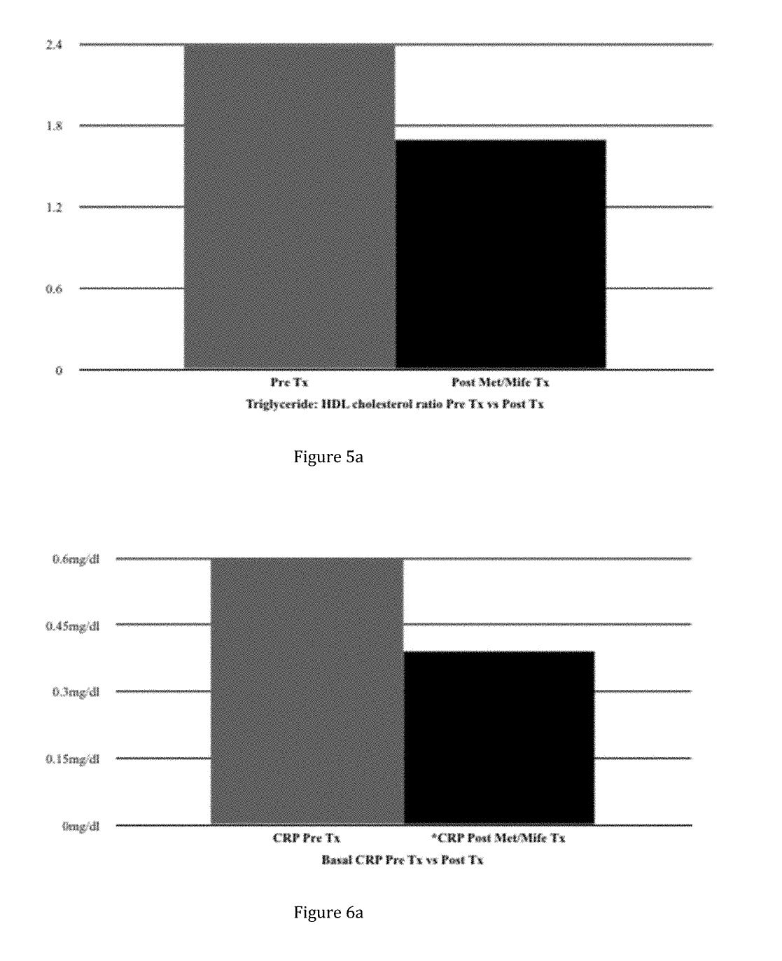

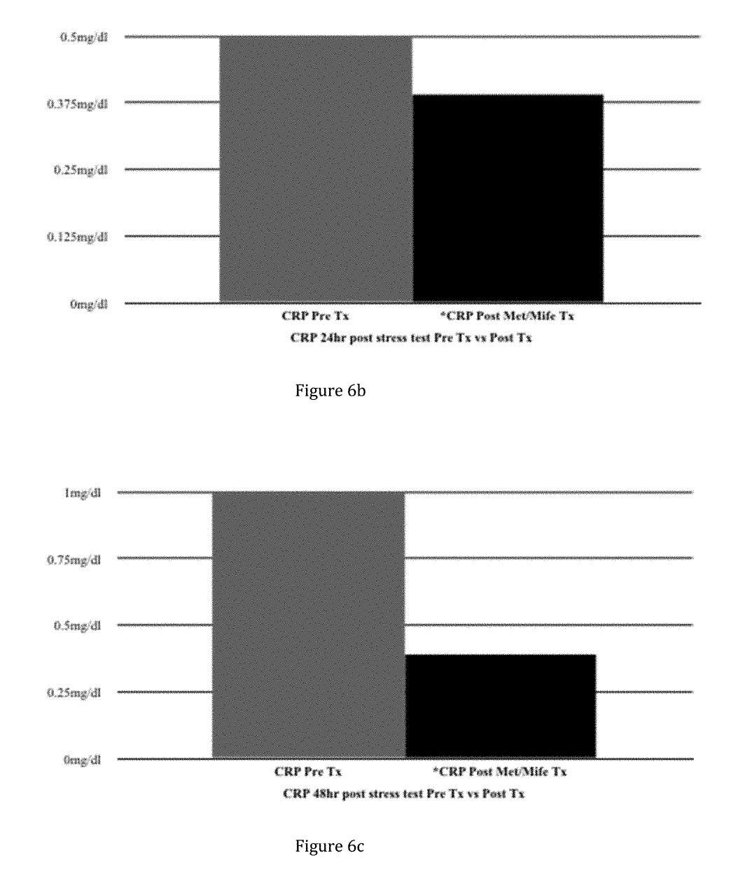

Results of Experiment 2 revealed that an exemplary embodiment of the present invention, namely the pharmaceutical composition [metformin/mifepristone], when administered to a human subject, demonstrated the effect of reducing CRP levels by a *minimum of 35%* for basal conditions relative to baseline CRP levels established for the human subject prior to treatment. (*all serum CRP measurements following treatment with [metformin/mifepristone] resulted in values below the laboratory detectable limit of 0.40 mg/dL, therefore a value of 0.39 mg/dL was used in order to provide a minimum level of change from untreated baseline values) (FIG. 6a). FIG. 6a shows significantly decreased serum C-reactive protein levels in a human subject following treatment with the pharmaceutical composition [metformin/mifepristone] compared to the baseline C-reactive protein level established for the human subject under basal conditions prior to treatment.

Results of Experiment 2 revealed that an exemplary embodiment of the present invention, namely the pharmaceutical composition [metformin/mifepristone], when administered to a human subject, demonstrated the effect of reducing CRP by a *minimum of 22%* 24 hours after an exercise to exhaustion stress test relative to CRP levels observed in an untreated human subject at 24 hours after an exercise to exhaustion stress test. (*all serum CRP measurements following treatment with [metformin/mifepristone] resulted in values below the laboratory detectable limit of 0.40 mg/dL, therefore a value of 0.39 mg/dL was used in order to provide a minimum level of change from non-treatment values) (FIG. 6b). FIG. 6b demonstrates significantly decreased serum C-reactive protein levels recorded in a human subject treated with the pharmaceutical composition [metformin/mifepristone] 24 hours after an exercise to exhaustion stress test compared to the serum C-reactive protein levels recorded in an untreated human subject 24 hours after an exercise to exhaustion stress test.

Results of Experiment 2 revealed that an exemplary embodiment of the present invention, namely the pharmaceutical composition [metformin/mifepristone], when administered to a human subject, demonstrated the effect of reducing the serum CRP by a* minimum of 61%* 48 hours after an exercise to exhaustion stress test relative to CRP levels observed in an untreated human subject 48 hour after an exercise to exhaustion stress test. (*all serum CRP measurements following treatment with [metformin/mifepristone] resulted in values below the laboratory detectable limit of 0.40 mg/dL, therefore a value of 0.39 mg/dL was used in order to provide a minimum level of change from untreated values) (FIG. 6c). FIG. 6c demonstrates significantly decreased serum C-reactive protein levels recorded in a human subject treated with the pharmaceutical composition [metformin/mifepristone] 48 hours after an exercise to exhaustion stress test compared to the serum C-reactive protein levels recorded in an untreated human subject 48 hours after an exercise to exhaustion stress test.

The prior art has identified urine lipid peroxides as an indicator or predictor or an indicator and predictor of bioenergetic dysfunction and/or oxidative stress associated diseases and/or disorders and/or conditions such as but not limited to; atherosclerosis, dyslipidemia, insulin resistance, obesity, metabolic syndrome, type 2 Diabetes Mellitus, inflammation and cancer (Tangvarasittichai S. Oxidative Stress, insulin resistance, dyslipidemia and type 2 diabetes mellitus; World J Diabetes. 2015 Apr. 15; 6(3): 456-480).

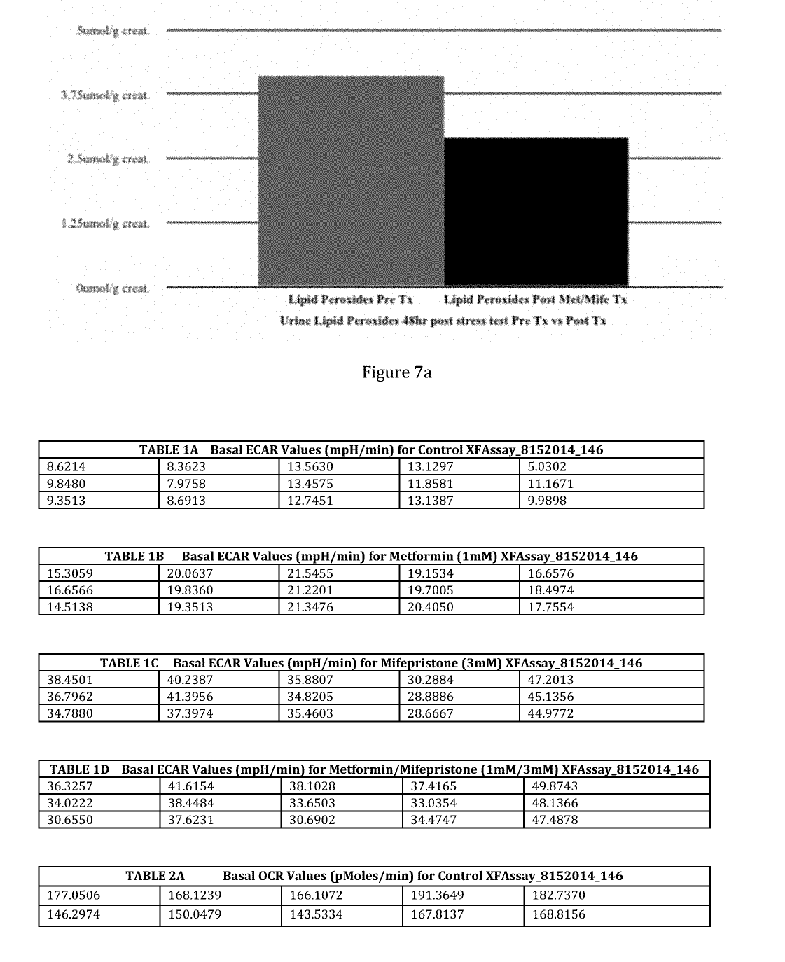

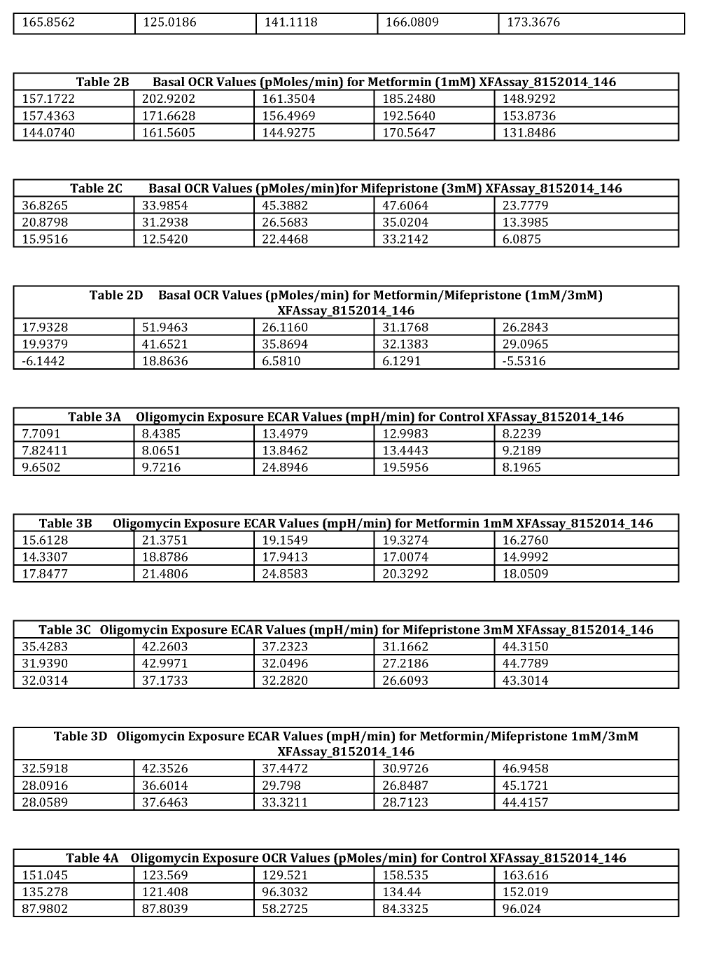

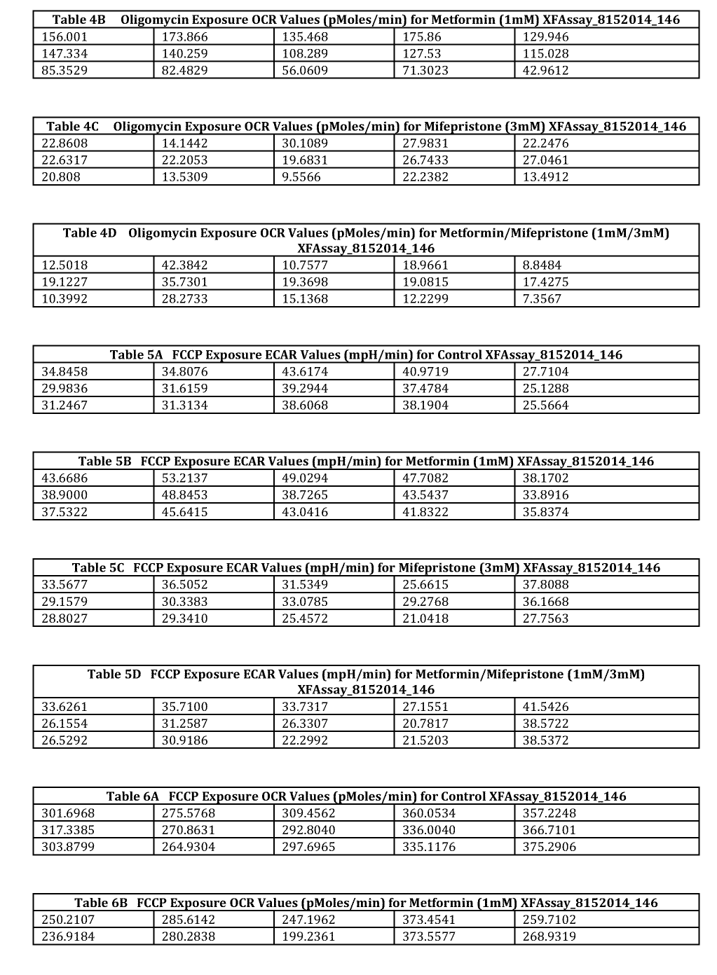

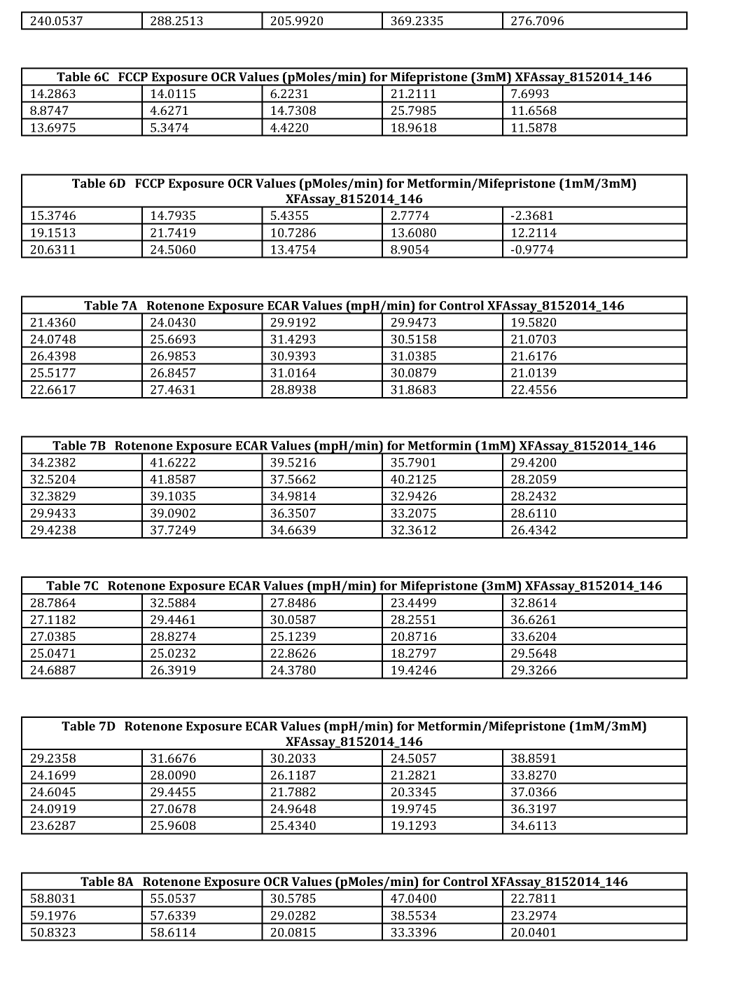

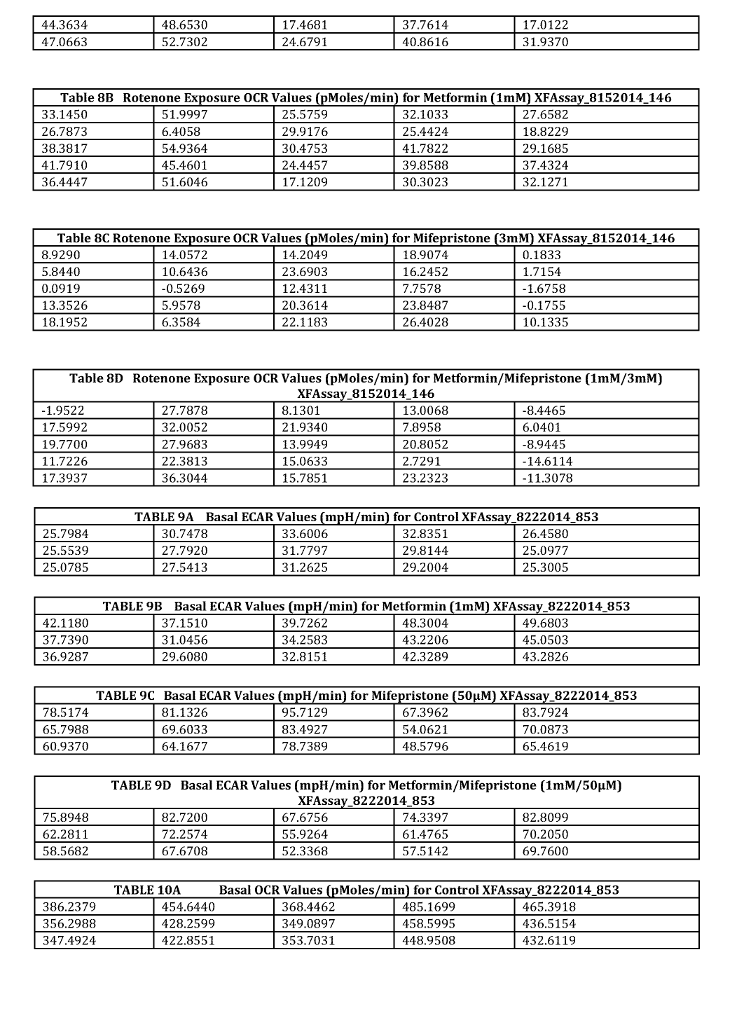

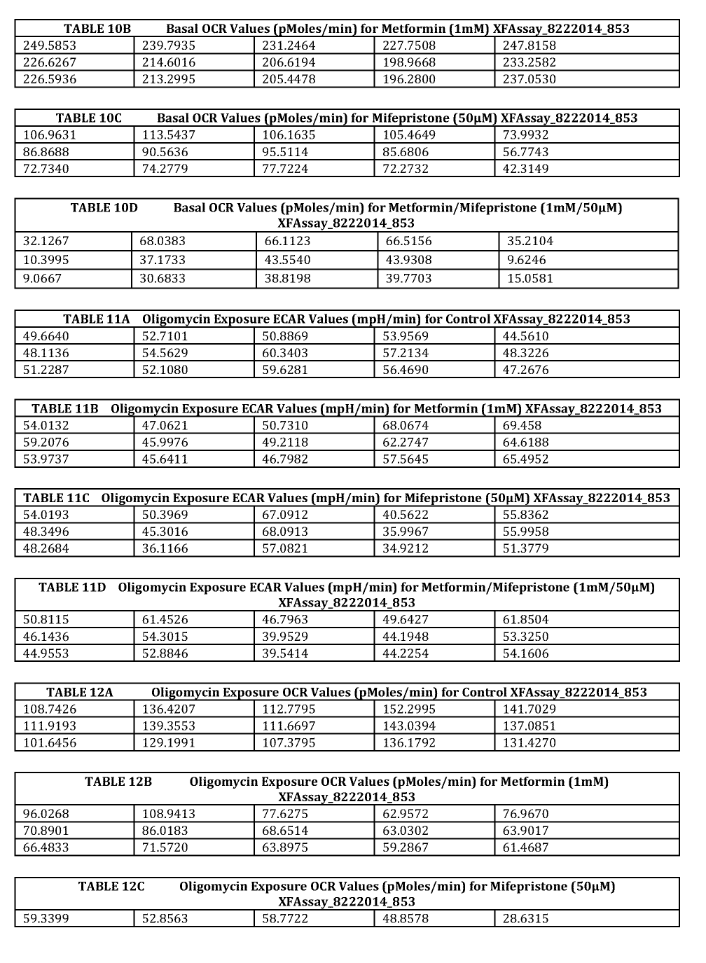

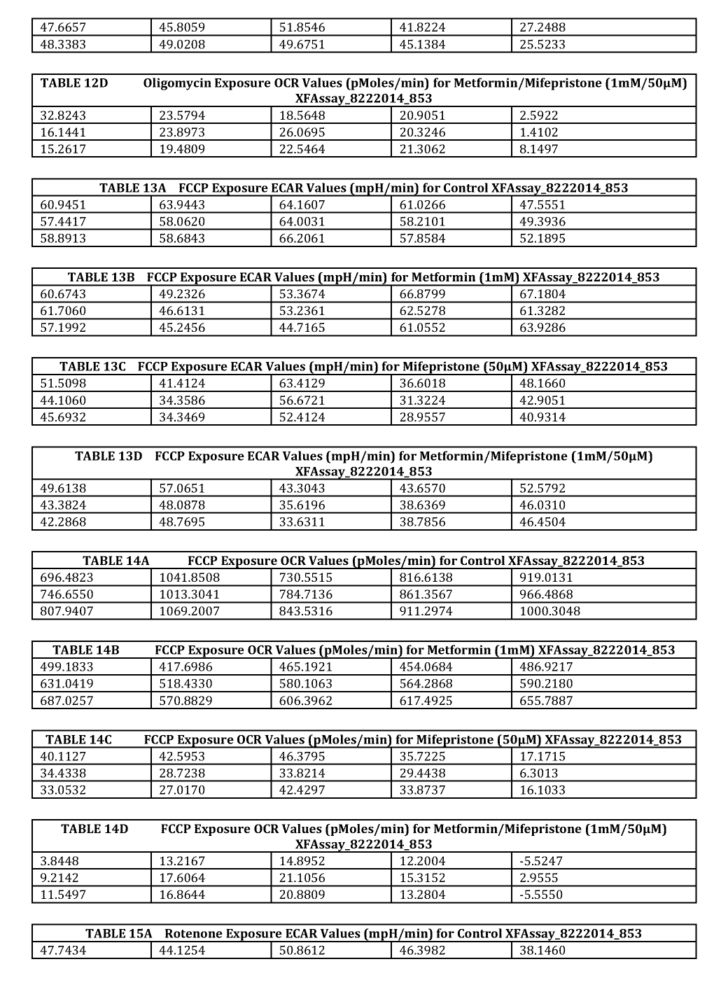

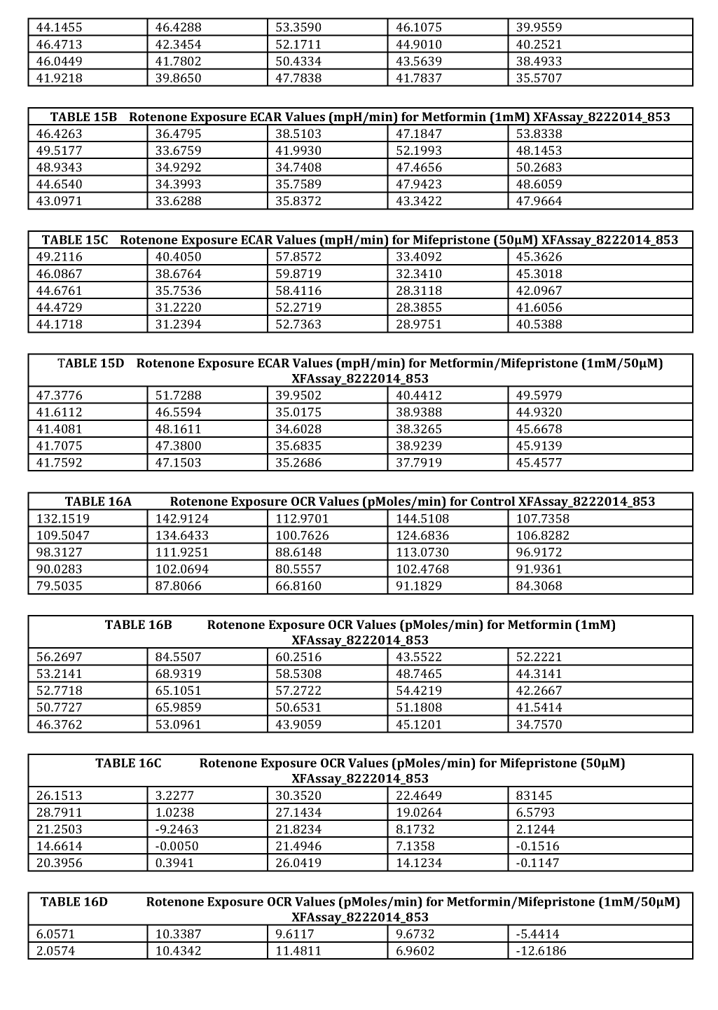

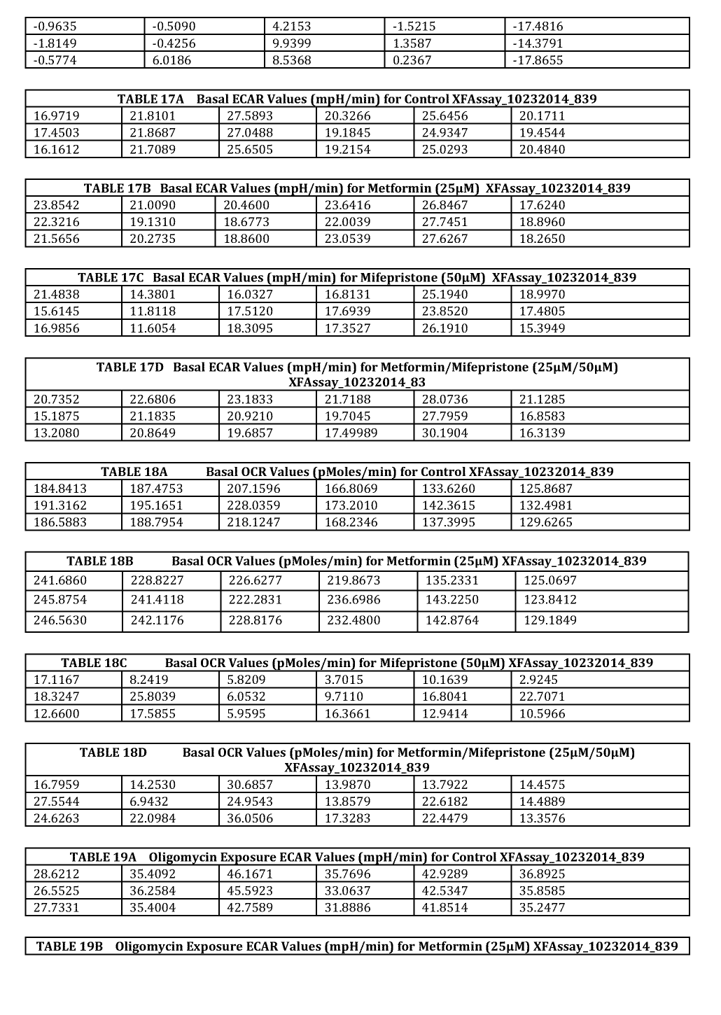

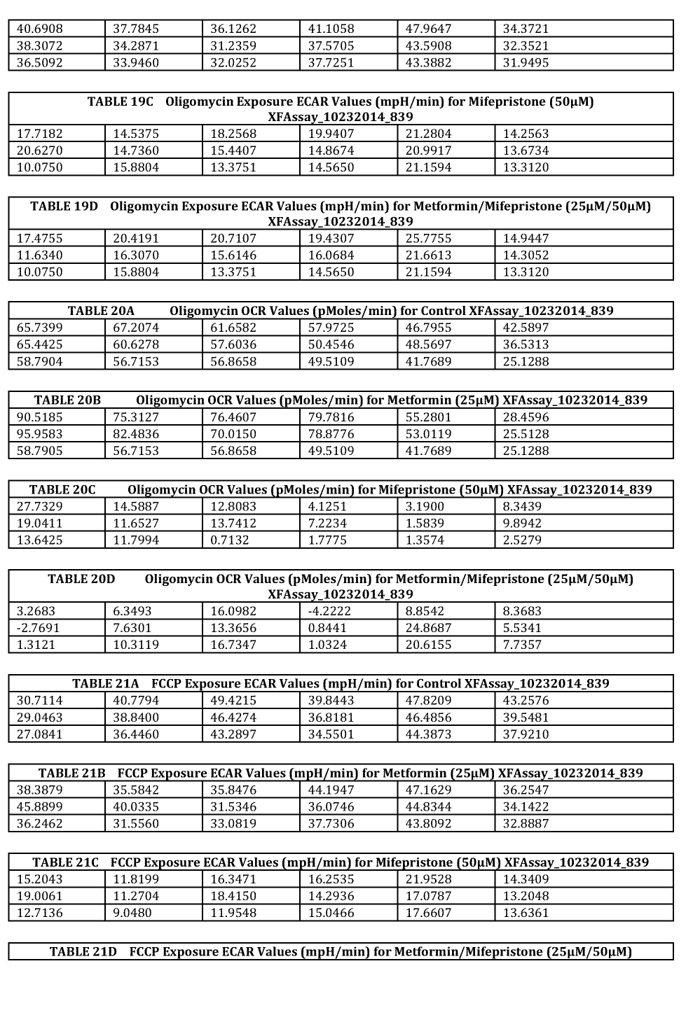

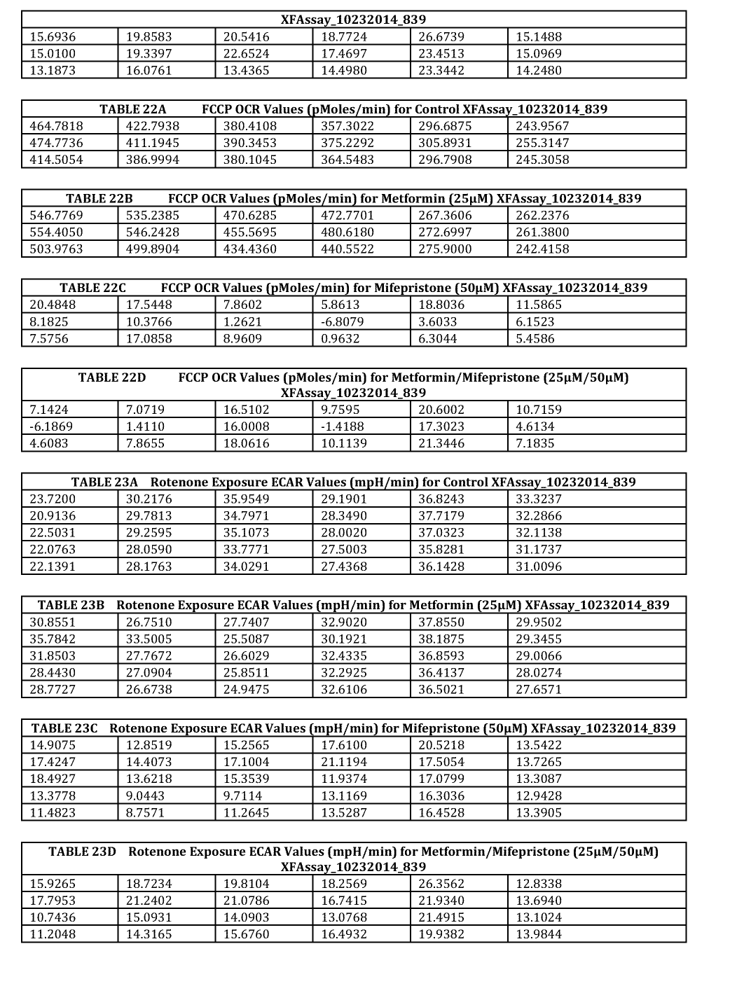

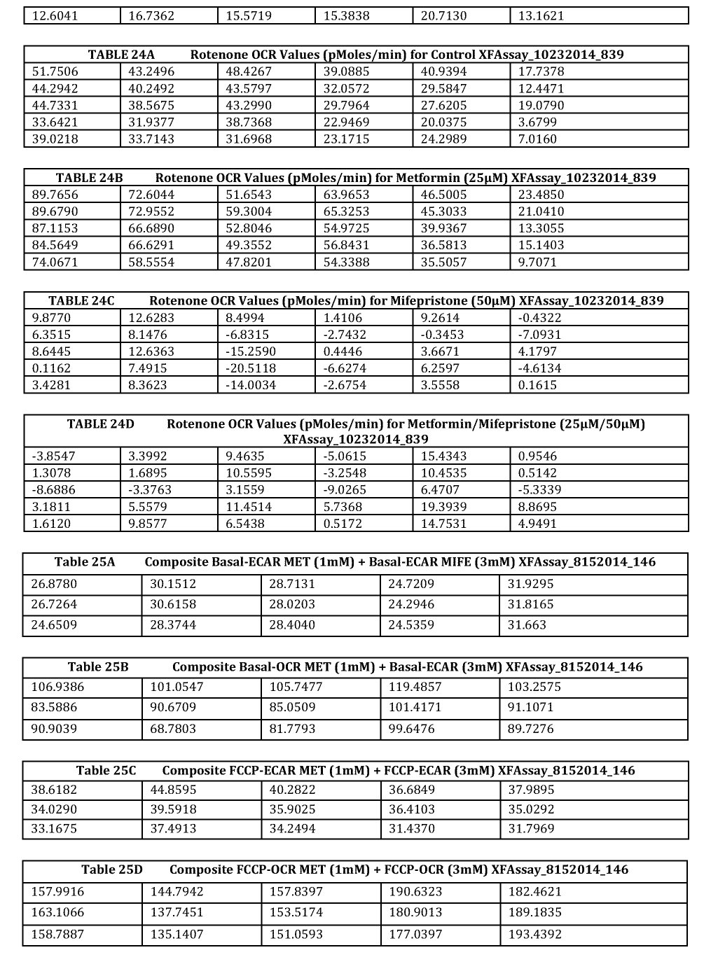

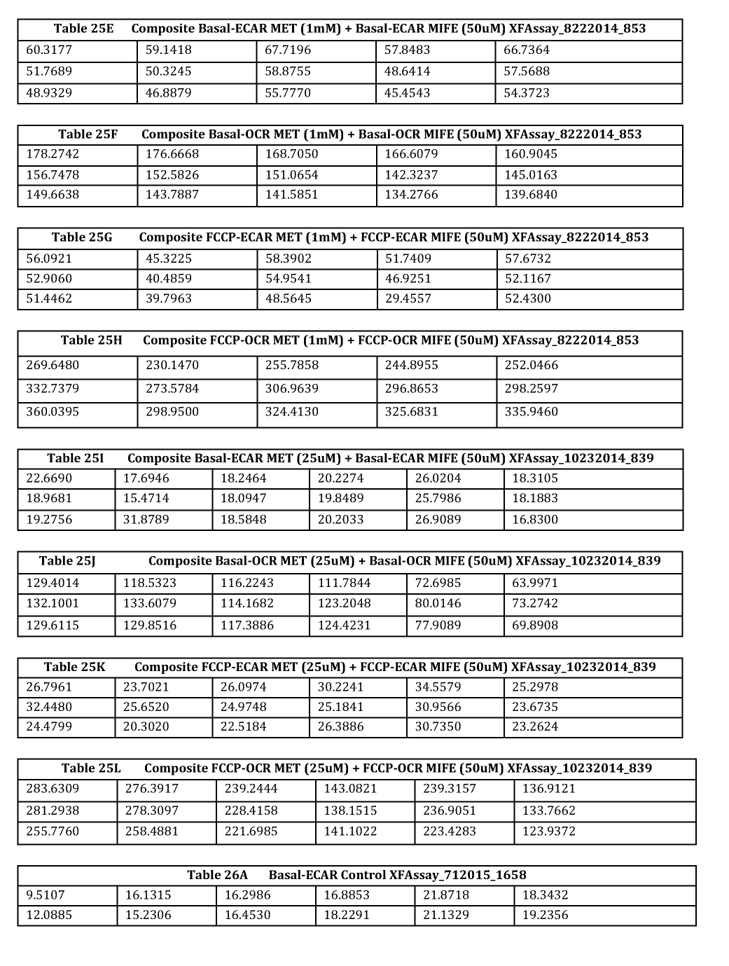

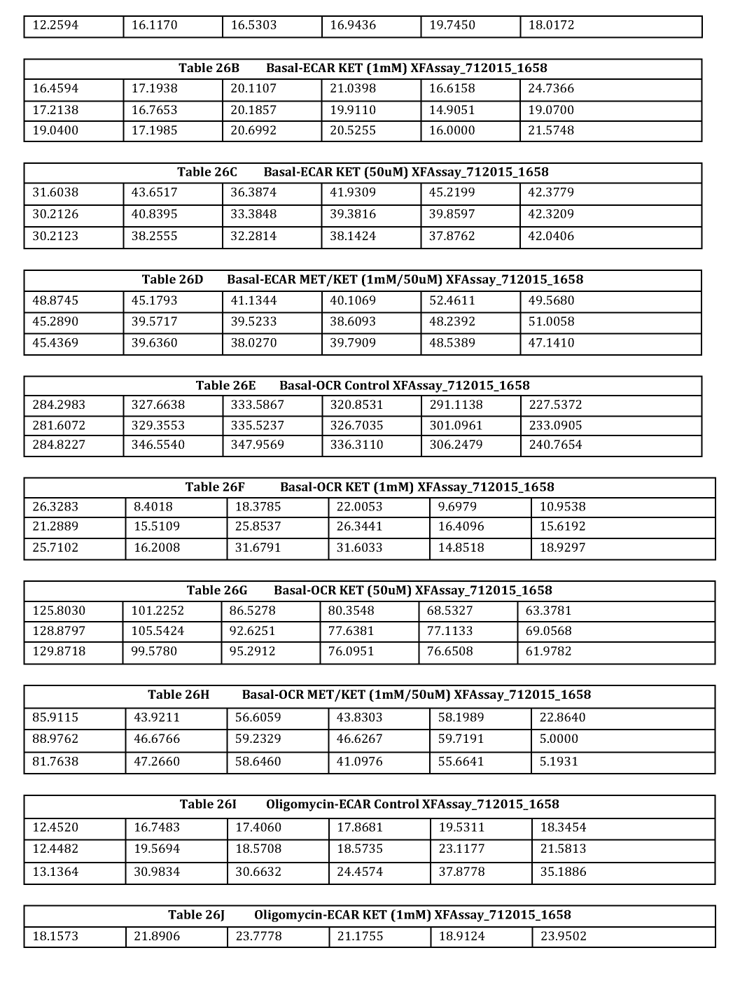

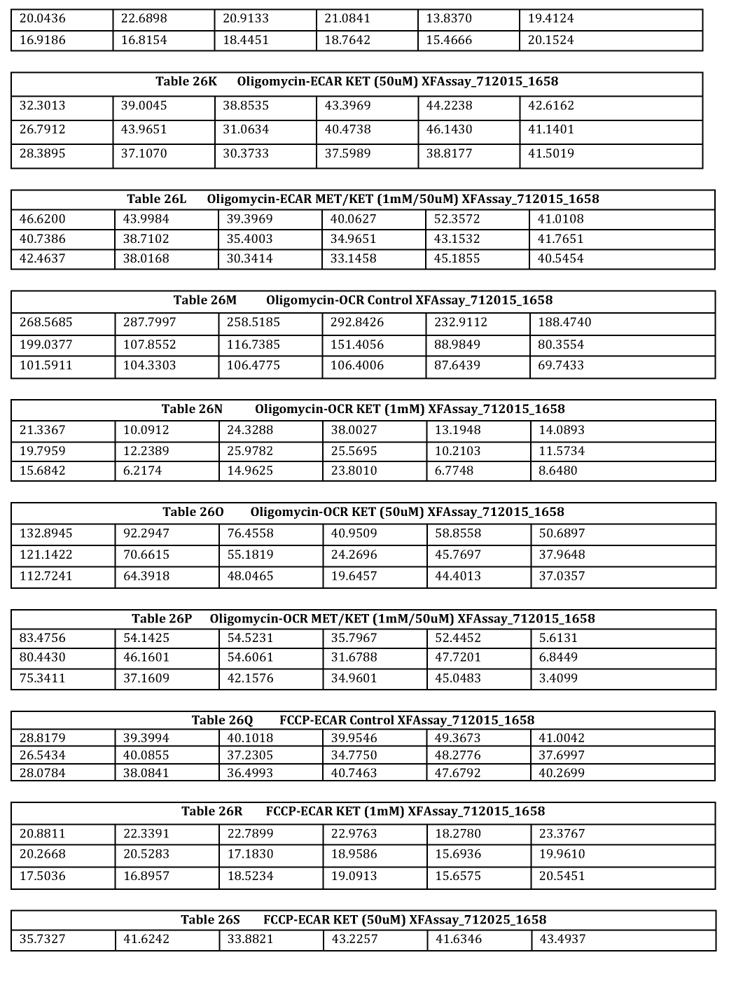

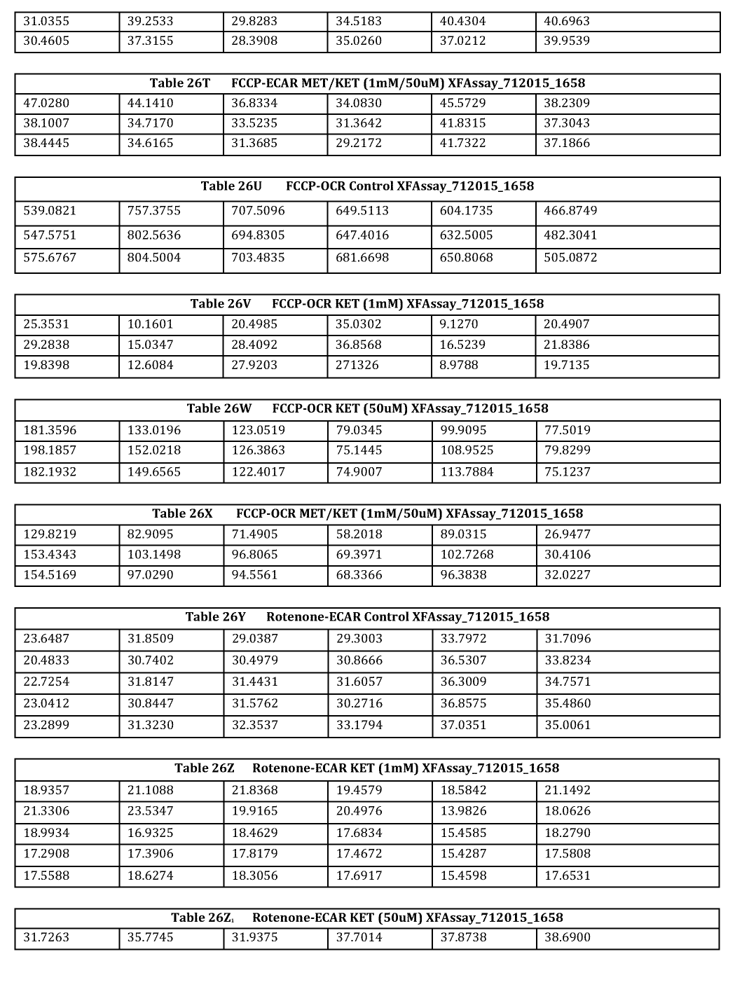

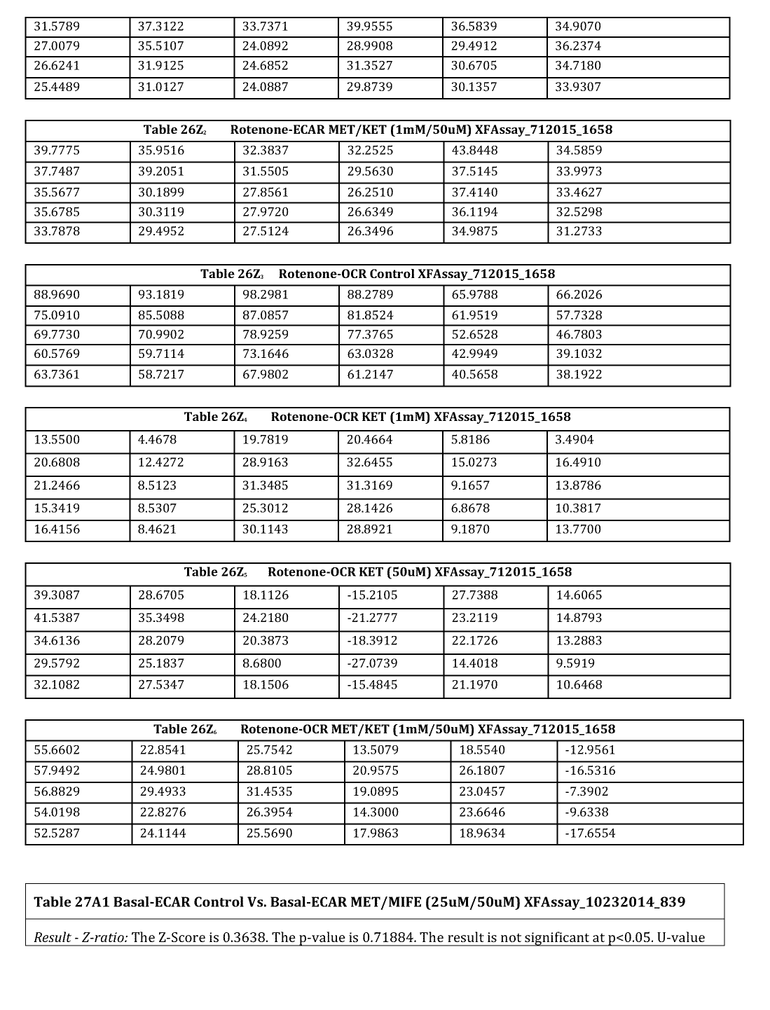

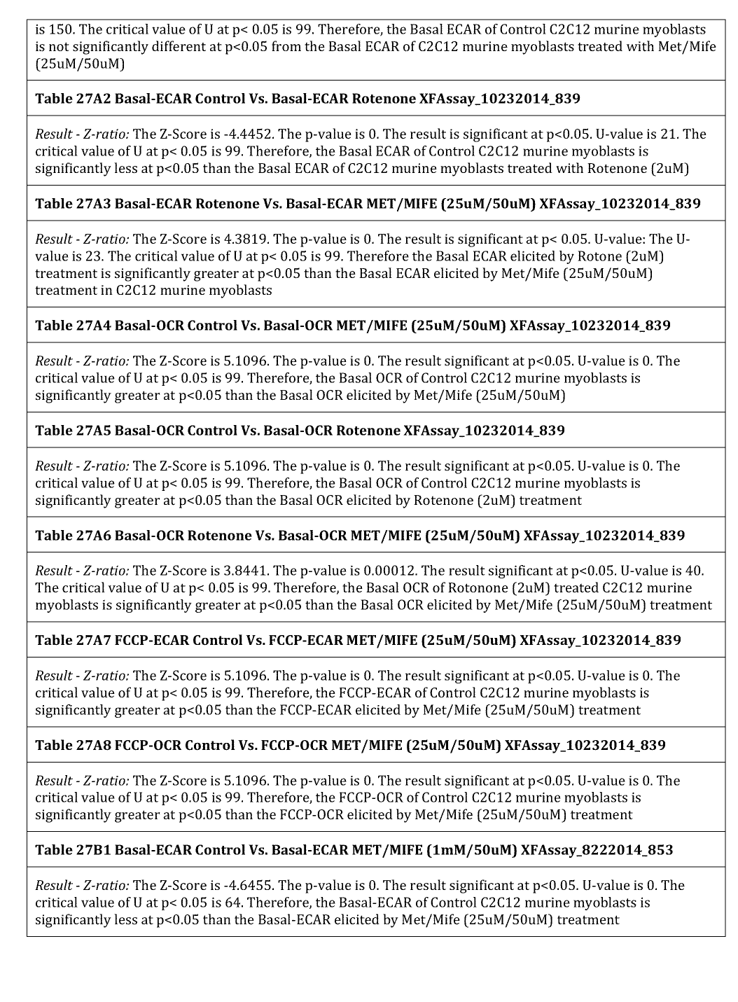

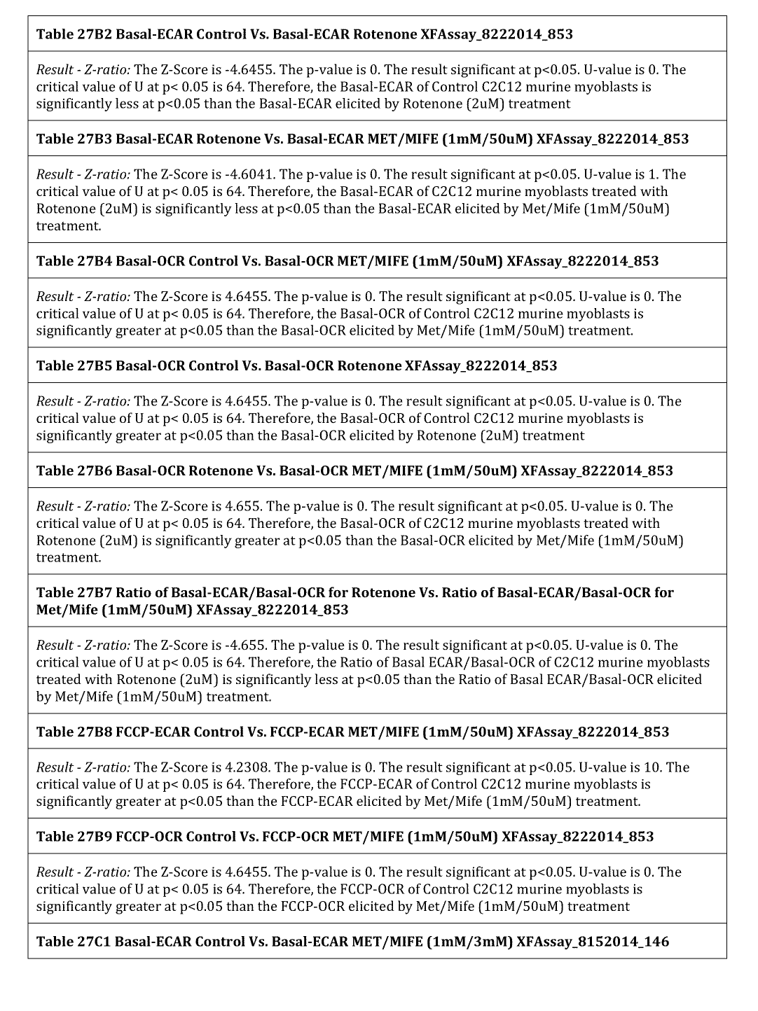

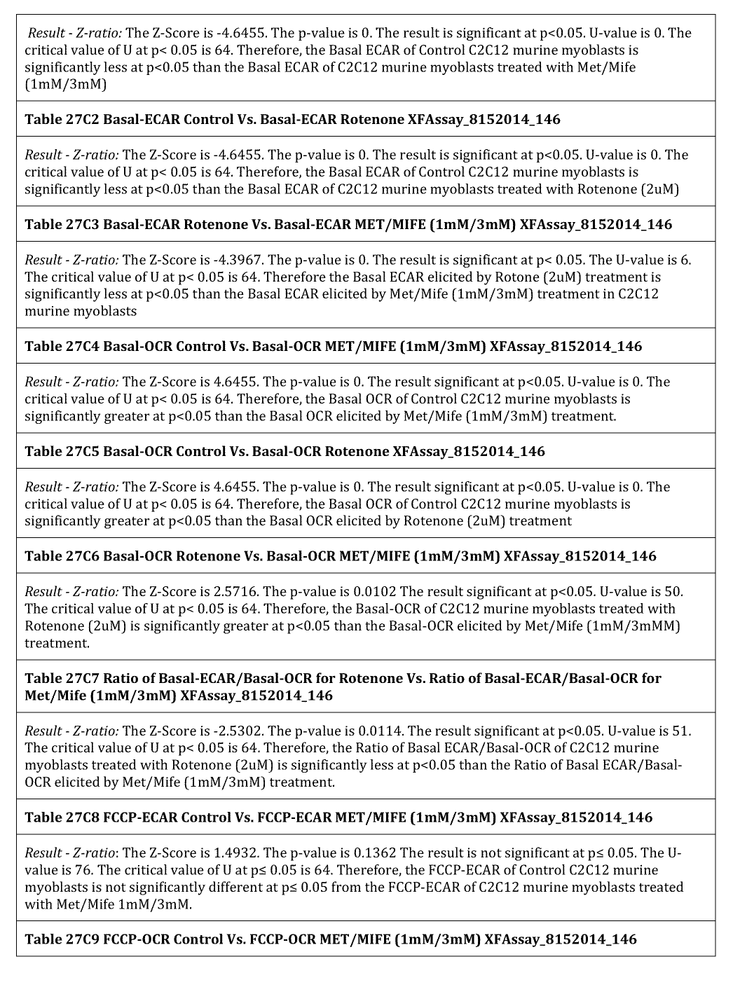

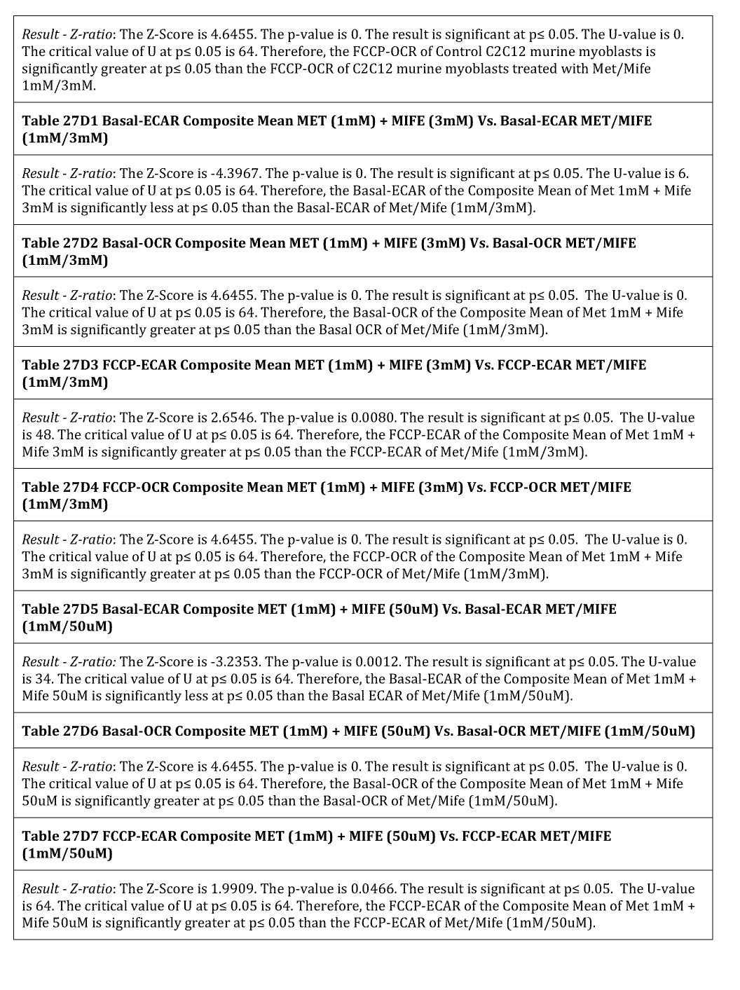

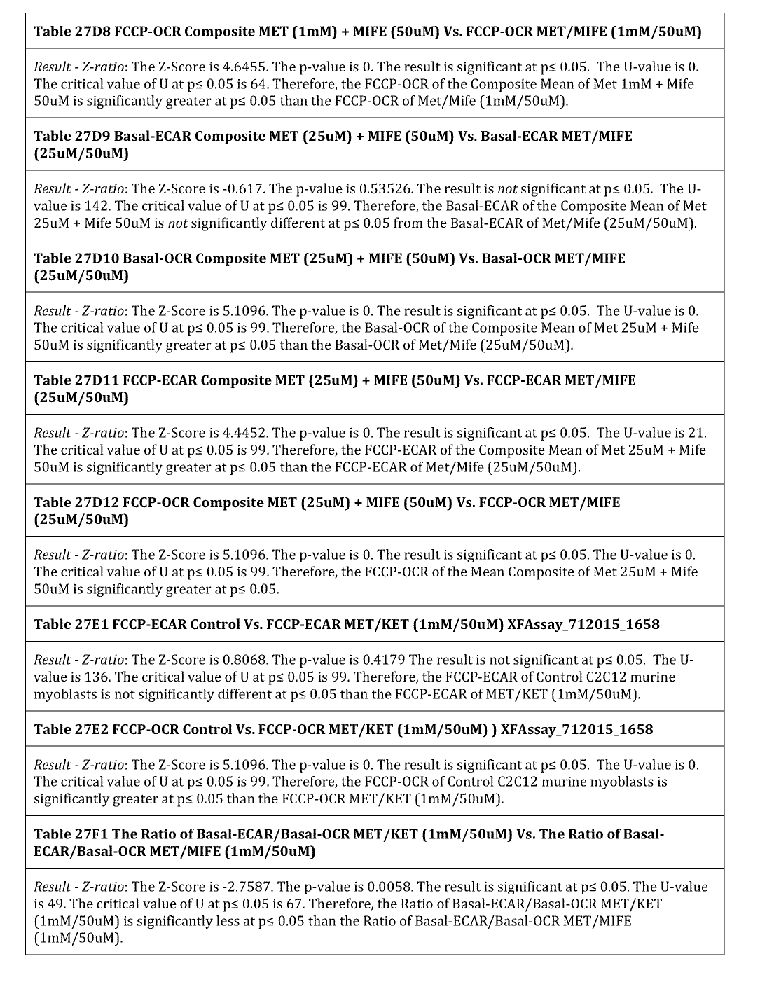

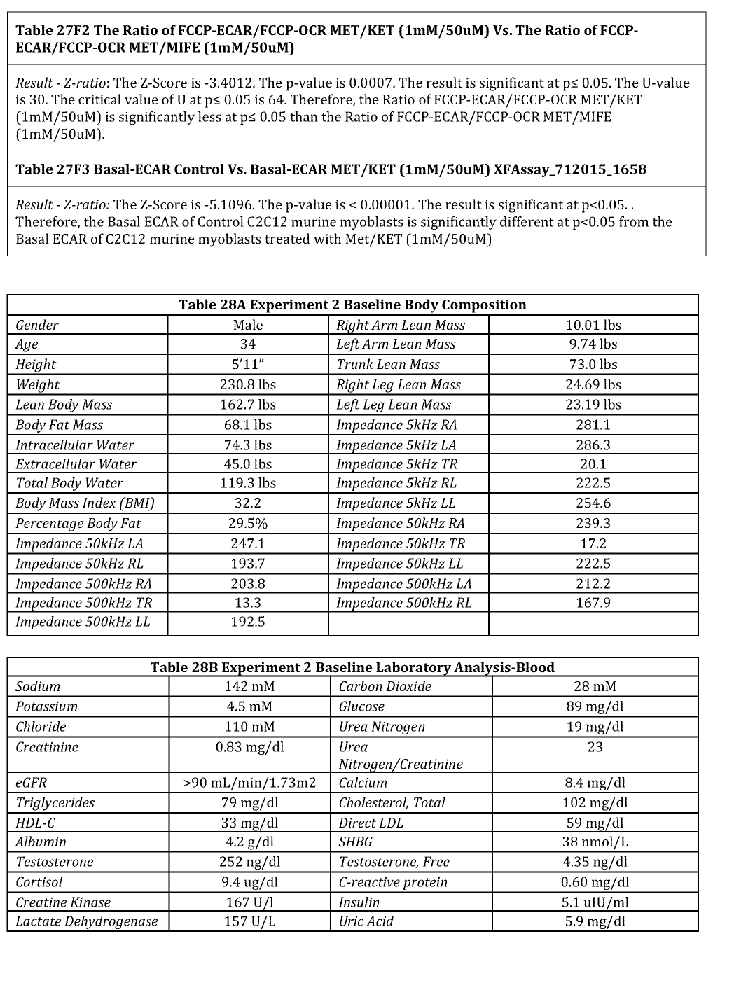

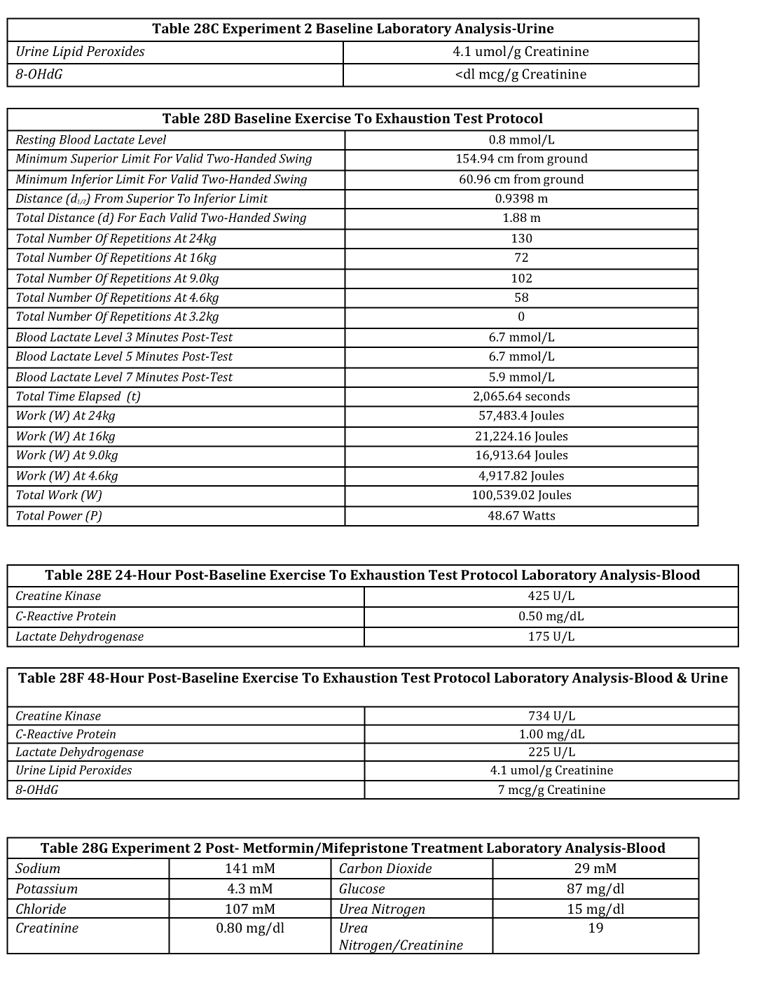

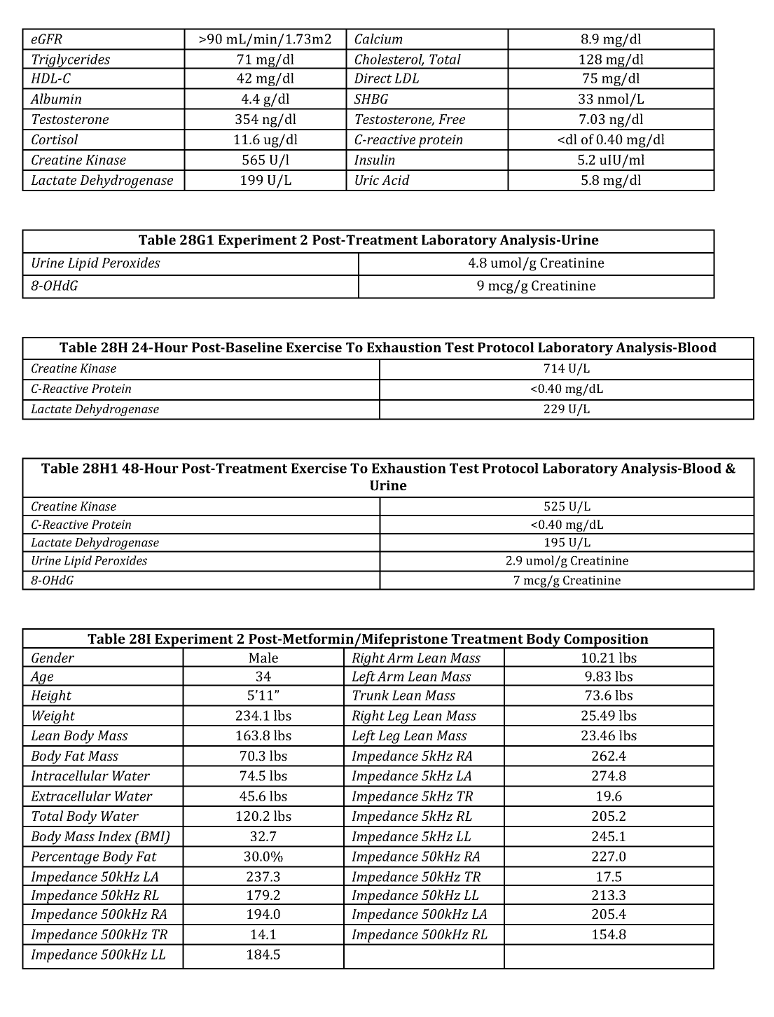

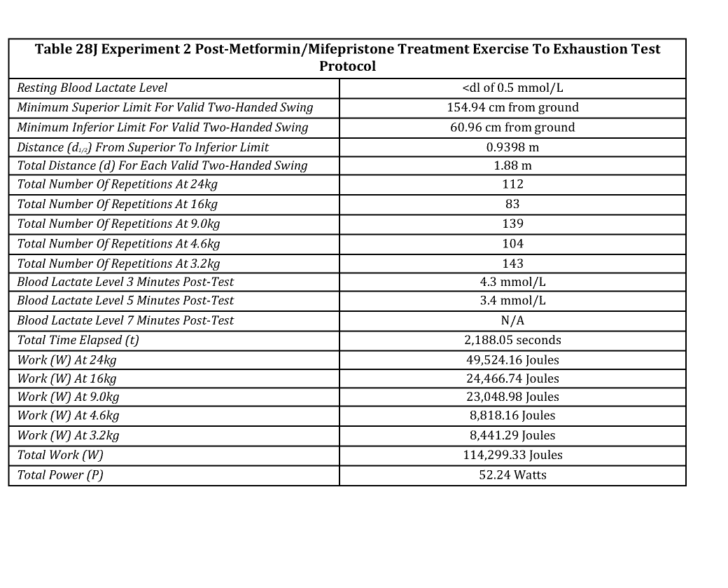

Results of Experiment 2 revealed that an exemplary embodiment of the present invention, consisting of the pharmaceutical composition [metformin/mifepristone], when administered to a human subject, demonstrated the effect of reducing the urine concentration of lipid peroxides at 48 hours after conducting an exercise to exhaustion stress test protocol by 39.6% relative to the urine concentration of lipid peroxides observed at 48 hours after an exercise to exhaustion stress test protocol in an untreated human subject (FIG. 7a). FIG. 7a demonstrates the significantly decreased urine lipid peroxide concentration observed in a human subject treated with the pharmaceutical composition [metformin/mifepristone] 48 hours after participating in an exercise to exhaustion stress test compared to the urine lipid peroxide concentration observed in an untreated human subject 48 hours after participating in an exercise to exhaustion stress test.

Diseases or disorders or conditions or diseases and disorders and conditions associated with bioenergetic dysfunction and/or oxidative stress for which embodiments of the present invention are preferred therapeutic agents for treatment or prevention or treatment and prevention include but are not limited to; the onset of aging or the progression of aging or the progression and onset of ageing, Alzheimer's disease, atherosclerosis, amyotrophic lateral sclerosis (ALS), acute alcoholic liver disease, adult respiratory distress syndrome (ARDS), ataxia telangiectasia (Louis-Bar syndrome), athracyline-related cardiomyopathy, cardiovascular disease, the cardio renal metabolic syndrome, cardiomyopathy, cardiotoxicity, cataract of the ocular lens, chronic kidney disease, chronic obstructive pulmonary disease (COPD), Crohn's disease, cancer, pre-cancer or metaplasia or genetic predisposition to cancer or cancer and pre-cancer and metaplasia and genetic predisposition to cancer, dementia, type 2 diabetes mellitus, Down's syndrome (Trisomy 21), Friedreich ataxia, heart failure, hepatotoxicity, hepatic cirrhosis, Hunington disease, hypercholesterolemia, hyperlipidemia, ischemia-reperfusion injury, interstitial lung disease, idiopathic pulmonary fibrosis, inflammation, ischemic injury, ischemic brain injury, microgravity, mitochondrial myopathy, myophosphorylase deficiency (McArdle's disease), multiple sclerosis, myocardial infarction, myocarditis, non-alcoholic fatty liver disease (NAFLD), non-alcoholic steatohepatitis (NASH), obesity, osteoarthritis, osteoporosis, pancreatitis, Parkinson's disease, primary billiary cirrhosis, preeclampsia, psoriasis, psoriatic arthritis, pulmonary hypertension, radiation sickness, reactive arthritis, rheumatoid arthritis, respiratory distress syndrome, sickle cell disease, spinal cord injury, sphereocytosis, systemic lupus erythematosus (SLE), systemic sclerosis, Werner syndrome, Zellweger syndrome, schizophrenia, depression, post traumatic stress disorder (PTSD), infectious diseases.

The embodiments of the present invention are particularly useful where generation of ROS contributes to oxidative stress and/or mitochondrial swelling and/or mitochondrial rupture and/or suppressed Lon protease activity and/or decreased ratio of NAD+ to NADH and/or suppressed Lon proteaste inducibility, such as is observed in ischemia/reperfusion injury and/or doxorubicin-induced cardiotoxicity (Weiss J N, et. al, Role of the mitochondrial permeability transition in myocardial disease. Circ Res. 2003 Aug. 22; 93(4):292-301), (Dirks-Naylor A J, et. al, The role of autophagy in doxorubicin-induced cardiotoxicity. Life Sci. 2013 Oct. 24. P-S0024-3205).

Cellular bioenergetic metabolism, the mitochondrial generation of ROS and oxidative stress play a determining role in the regulation of cell cycle progression in eukaryotic biological systems. Therefore, embodiments of the present invention may be applied to biological systems including but not limited to, organelle populations or cell populations or tissue populations or organ populations or organ system populations or organism populations or organelle populations and cell populations and tissue populations and organ populations and organ system populations and organism populations utilized in engineering processes.

In some embodiments of the invention an active principal is an agent that is an inhibitor of a mitochondrial ROS generating process and the agent inhibits mitochondrial NADH-coenzyme Q oxidoreductase (complex I).

In an exemplary embodiment of the invention, an active principal that reduces the activity of mitochondrial NADH-coenzyme Q oxidoreductase (complex I) is a biguanide (BG).

The term “biguanide” or “biguanide agent” or “BG” or “BG agent” is a term of art and refers to molecular compounds or molecular structures or molecular compounds and molecular structures that are based on the structural formula for a biguanide or related heterocyclic compounds, such as but not limited to those disclosed by WO2013103384 A1 paragraphs 0009 through 0028, paragraphs 0091 through 0122 and paragraphs 0126 through 0131, and U.S. Pat. No. 2,961,377 A column 2, line 35 through column 3, line 28 and column 3, line 62 through column 4, line 14 which are incorporated herein by reference. Whereas WO2013103384 A1, paragraphs 0123 through 0125, and U.S. Pat. No. 2,961,377 A column 3, line 29 through column 3 line 62 and column 4, line 15 through column 7, line 75, disclose techniques and methods for the synthesis of BG agents and are incorporated herein by reference.

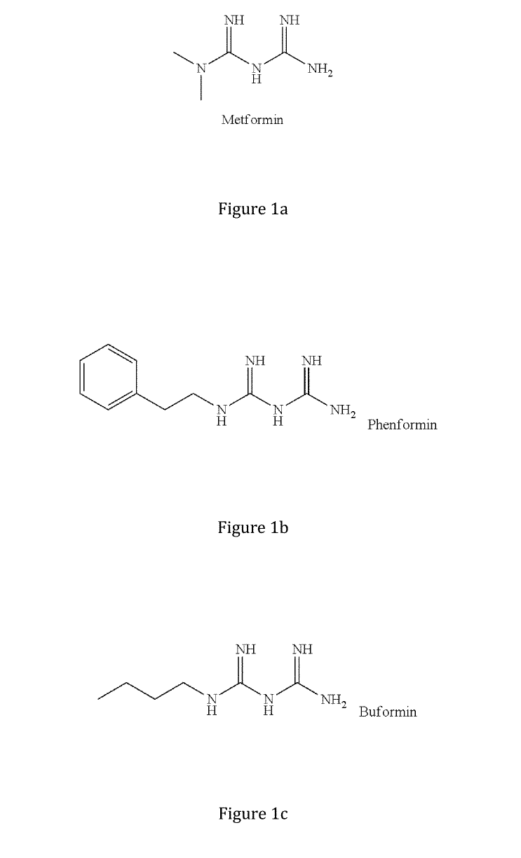

In an exemplary embodiment of the invention the active principal agent that reduces the activity of a mitochondrial ROS generating process is an agent that reduces the activity of mitochondrial NADH-coenzyme Q oxidoreductase (complex I) and; the agent is the biguanide metformin (FIG. 1a). FIG. 1a shows the general molecular structure of metformin.

The term “metformin” or “met” or “3-(diaminomethylidene)-1,1-dimethylguanidine” or “dimethylbiguanidine”, as used herein, is a term of art and means molecular compounds or molecular structures or molecular compounds and molecular structures that are BG agents described by simplified molecular-input line-entry system (SMILES) as; CN(C)C(═N)N═C(N)N and; metabolites and/or prodrugs and/or conjugates and/or other such derivatives and/or analogs and/or related compounds of the molecular structure and/or the molecular compound described by simplified molecular-input line-entry system (SMILES)as; CN(C)C(═N)N═C(N)N.

In yet another exemplary embodiment of the invention the active principal agent that reduces the activity of a mitochondrial ROS generating process is an agent that reduces the activity of mitochondrial NADH-coenzyme Q oxidoreductase (complex I) and; the agent is the biguanide phenformin (FIG. 1b). FIG. 1b shows the general molecular structure of phenformin.

The term “phenformin” or “1-(diaminomethylidene)-2-(2phenylethyl)guanidine”, as used herein, is a term of art and means molecular compounds or molecular structures or molecular compounds and molecular structures that are BG agents described by simplified molecular-input line-entry system (SMILES)as; C1=CC═C(C═C1)CCN═C(N)N═C(N)N and; metabolites and/or prodrugs and/or conjugates and/or other such derivatives and/or analogs and/or related compounds of the molecular structure and/or the molecular compound described by simplified molecular-input line-entry system (SMILES) as; C1=CC═C(C═C1)CCN═C(N)N═C(N)N and includes 4-hydroxyphenformin, described by simplified molecular-input line-entry system (SMILES)as; C1=CC(═CC═C1CCN═C(N)N═C(N)N)O and; metabolites and/or prodrugs and/or conjugates and/or other such derivatives and/or analogs and/or related compounds of the molecular structure and/or the molecular compound described by simplified molecular-input line-entry system (SMILES) as; C1=CC(═CC═C1CCN═C(N)N═C(N)N)O.

In yet another exemplary embodiment of the invention the active principal agent that reduces the activity of a mitochondrial ROS generating process is an agent that reduces the activity of mitochondrial NADH-coenzyme Q oxidoreductase (complex I) and; the agent is the biguanide buformin (FIG. 1c). FIG. 1c shows the general molecular structure of buformin.

The term “buformin” or “1-butylbiguanide” or “2-butyl-1-(diaminomethylidene) guanidine”, as used herein, is a term of art and means molecular compounds and/or molecular structures that are described by simplified molecular-input line-entry system (SMILES) as; CCCCNC(═N)NC(═N)N and; metabolites and/or prodrugs and/or conjugates and/or other such derivatives and/or analogs and/or related compounds of the molecular structure and/or the molecular compound described by simplified molecular-input line-entry system (SMILES)as; CCCCNC(═N)NC(═N)N.

In an exemplary embodiment of the present invention the active principal that contributes to a reduced rate of mitochondrial oxygen consumption is mifepristone. This compound and methods for its preparation are described in CN1218665 A, EP1990044 A1, and are herein incorporated in their entirety by reference.

The term “mifepristone” or “mife” or “RU486”, as used herein, is a term of art and means the family of molecular compounds or molecular structures or molecular compounds and molecular structures described by simplified molecular-input line-entry system (SMILES) as; O═C5\C═C4/C(═C3/[C@@H](c1ccc(N(C)C)cc1)C[C@]2([C@@H(CC[C@]2(C#CC)O)[C@@H]3CC4)C)CC5 and referred to as RU38486, or RU42633 or RU42698 or 17-(3-hydroxy-11-(3-(4-dimethyl-aminophenyl)-17-a-(1-propynyl)-estra-4,9-dien-3-one) or 11-(3-(4dimethylaminophenyl)-17-(3-hydroxy-17-a-(1-propynyl)-estra-4,9-dien-3-one), or 11(3-[p-(Dimethylamino)phenyl]-17(3-hydroxy-17-(1-propynyl)-estra-4,9-dien-3-one or 11(3-(4-dimethyl-aminophenyl)-17(3-hydroxy-17a-(prop-1-ynyl)-estra-4,9-dien-3-one or 17(3-hydroxy-11(3-(4-dimethylaminophenyl-1)-17a-(propynyl-1)-estra-4,9-diene-3-one or 17(3-hydroxy-11(3-(4-30 dimethylaminophenyl-1)-17a-(propynyl-1)-E or (11(3,17(3)-11-[4-dimethylamino)-phenyl]-17-hydroxy-17-(1-propynyl)estra-4,9-dien-3-one or and 11[3-[4-(N,N-dimethylamino) phenyl]-17a-(prop-1-ynyl)-D-4,9-estradiene-17(3-ol-3-one) or 17beta-Hydroxy-11beta-[4-(methylamino)-phenyl]-17alpha-(1-propinyl)-estra-4,9-dien-3-one or RU38486 and RU42633 and RU42698 and 17-(3-hydroxy-11-(3-(4-dimethyl-aminophenyl)-17-a-(1-propynyl)-estra-4,9-dien-3-one) and 11-(3-(4dimethylaminophenyl)-17-(3-hydroxy-17-a-(1-propynyl)-estra-4,9-dien-3-one) and 11(3-[p-(Dimethylamino)phenyl]-17(3-hydroxy-17-(1-propynyl)-estra-4,9-dien-3-one and 11(3-(4-dimethyl-aminophenyl)-17(3-hydroxy-17a-(prop-1-ynyl)-estra-4,9-dien-3-one and 17(3-hydroxy-11(3-(4-dimethylaminophenyl-1)-17a-(propynyl-1)-estra-4,9-diene-3-one and 17(3-hydroxy-11(3-(4-30 dimethylaminophenyl-1)-17a-(propynyl-1)-E and (11(3,17(3)-11-[4-dimethylamino)-phenyl]-17-hydroxy-17-(1-propynyl)estra-4,9-dien-3-one and 11[3-[4-(N,N-dimethylamino) phenyl]-17a-(prop-1-ynyl)-D-4,9-estradiene-17(3-ol-3-one) and 17beta-Hydroxy-11beta-[4-(methylamino)-phenyl]-17alpha-(1-propinyl)-estra-4,9-dien-3-one or analogs thereof and; metabolites and/or prodrugs and/or conjugates and/or other such derivatives and/or analogs and/or related compounds of the molecular structure and/or the molecular compound described by simplified molecular-input line-entry system (SMILES) as; O═C5\C═C4/C(═C3/[C@@H](c1ccc(N(C)C)cc1)C[C@]2([C@@H(CC[C@]2(C#CC)O)[C@@H]3CC4)C)CC5.

In an exemplary embodiment of the invention the composition contains at least one active principal as a biguinide, selected from the group: metformin, phenformin, buformin and at least one active principal as mifepristone.

In an exemplary embodiment of the invention the pharmaceutical composition contains the active principals metformin and mifepristone.

In another exemplary embodiment of the invention the pharmaceutical composition contains the active principals phenformin and mifepristone.

In yet another exemplary embodiment of the invention the pharmaceutical composition contains the active principals buformin and mifepristone.

Dosages, Administration and Pharmaceutical Compositions:

The choice of appropriate active principal agents used in embodiments of the present invention can be determined and optimized upon identifying the condition to be treated and the desired therapeutic outcome.

Embodiments of the present invention intended to treat or prevent or treat and prevent diseases, disorders and conditions associated with oxidative stress feature active principal agents selected based on factors such as but not limited to; therapeutic potency, defined herein as the resultant inhibition of anaerobic metabolism in a eukaryotic biological system per unit mass of the embodiment of the present invention administered to the eukaryotic biological system and/or the target of bioaccumulation for administered active principal agents of the embodiment of the present invention.

The choice of appropriate dosages for the active principals used to comprise the embodiments of the present invention can be determined or optimized or determined and optimized by the skilled artisan through observation of the patient and/or subject, including the overall health of the subject or diagnostic bio-markers or the response to therapy or the presence of genetic polymorphisms that influence therapeutic response or absence of genetic polymorphisms that influence therapeutic response or the overall health of the subject and diagnostic bio-markers and the response to the therapy and the presence of genetic polymorphisms that influence therapeutic response and the absence of genetic polymorphisms that influence therapeutic response.

Optimization, for example, may be necessary if it is determined that a subject and/or patient is not exhibiting the desired therapeutic effect or if the subject or patient is experiencing adverse effects or if the subject or patient is not exhibiting the desired therapeutic effect and the subject or patient is experiencing adverse effects.

Preferably, an active principal agent that is an inhibitor of mitochondrial ROS generating processes, such as the Complex I inhibitor metformin, is prescribed at a dosage equal to or higher than the maximal dosage routinely used by a skilled artisan to elicit the desired therapeutic effect of the active principal agent, when the active principal agent is used as a monotherapy.

Embodiments of the present invention allow for the dosing of active principal agents, such as the biguanide agent metformin, at higher doses than routinely used by a skilled artisan as a result of the unexpected and novel emergent properties characteristic of embodiments of the present invention which elicit lower than expected levels of anerobic metabolic activity in eukaryotic biological systems treated with embodiments of the present invention.

Lactic acidosis is well known side effect of treatment with biguanide agents, with concerns over the development of lactic acidosis leading to the removal of the biguanide agents phenformin and buformin from use as therapeutic agents by regulatory authorities.

Embodiments of the present invention demonstrate the characteristic trait of reducing and/or eliminating the increase in blood lactate traditionally associated with biguanide use in the prior art. (FIG. 3a, 3b, 3c)

A biguanide agent may be prescribed, for example, at a dose of 5 mg to 3000 mg daily.

In an embodiment of the present invention, wherein the one of the active principal agents is mifepristone, the dose is at least 5 mg daily, and should be less than 1200 mg daily or 20 mg/kg of total subject mass, whichever is less.

Preferably, the dose should be in the range of about 10 mg to 800 mg daily, more preferably in the range of about 20 mg to 600 mg daily, and optimally in the range of about 25 mg to 200 mg daily.

The term “dose” as used herein, means an amount or form or amount and form of a pharmaceutical composition administered to a subject, typically after titrating the dose from a lower concentration starting dose, over a period of time on the order of days to several weeks.

It is advantageous to formulate compositions of the invention in unit dosage forms for ease of administration or uniformity of dosage or ease of administration and uniformity of dosage.

The term “unit dosage forms” as used herein, means physically discrete units suited as unitary dosages for administration to individual subjects. That is, the compositions are formulated into discrete dosage units each containing a predetermined, “unit dosage” quantity of an active principal or active principals calculated to produce the desired therapeutic effect or unit dosage quantity of an active principal and an amount of active principal calculated to produce the desired therapeutic effect in association with the required pharmaceutically acceptable carrier.

The specifications of the unit dosage forms of the invention are dependent on the unique characteristics of the composition containing the active principal agents. It is also within the scope of the embodiments of the present invention to formulate a single physically discrete dosage form containing each of the active principal agents of the composition.

The method of administering embodiments of the invention in the form of pharmaceutical compositions will depend, in particular, on the type of active principal agents selected. The active principal agents may be administered together in the same composition or simultaneously as separate compositions or sequentially as separate compositions or together in the same composition and simultaneously as separate compositions and sequentially as separate compositions.

In yet another aspect, some embodiments of the present invention agents may be administered at different times of day, with the either of the pharmaceutical compositions two minimum active principals administered separately or sequentially or separately and sequentially. In some embodiments of this invention, the minimum of two active principal agents are administered simultaneously using one or more dosage forms.

Active principal agents can also be administered along with a pharmaceutically acceptable carrier. As used herein “pharmaceutically acceptable carrier” includes any solvents, dispersion media, coatings, antibacterial agents, antifungal agents, isotonic agents, absorption delaying agents, and the like. The use of such media or agents or media and agents for pharmaceutical compositions is well known in the art. Except insofar as any media or agent or media and agent is incompatible with the active principal agent or active principal agents, use thereof in compositions of the invention is contemplated.

Of course, any pharmaceutically acceptable carrier used in preparing any dosage unit form should be pharmaceutically pure and substantially non-toxic in the amounts employed, and can also be administered in a convenient manner such as by injection (subcutaneous, intravenous, etc.), inhalation, transdermal application, sub-dermal implant, tissue implant, oral suspension or rectal administration.

An active principal agent alone, or in combination with another active principal agent, in the form of a composition, when orally administered to a subject, may include an inert diluent or an assimilable edible carrier or an inert diluent and an assimilable edible carrier.

Pharmaceutically acceptable diluents include but are not limited to saline or aqueous buffer solutions or saline and aqueous buffer solutions. Liposomes include but are not limited to water-in-oil-in-water CGF emulsions as well as conventional liposomes. To administer the embodiments of the invention as pharmaceutical compositions containing an active principal agent that inhibits mitochondrial ROS generating process and a second active principal agent that contributes to a reduced rate of mitochondrial oxygen consumption parenterally or intraperitoneally, dispersions can be prepared in glycerol, liquid polyethylene glycols, and mixtures thereof and in oils. Under ordinary conditions of storage and use, these preparations may contain a preservative to prevent the growth of microorganisms.

Compositions suitable for injectable use include sterile aqueous solutions (where water-soluble), sterile oil suspensions, sterile emulsions or sterile dispersions and sterile powders for the extemporaneous preparation of sterile injectable solutions or sterile injectable dispersions.

In all cases, the composition must be sterile and upon administration must be fluid to the extent that easy syringeability exists. It must be stable under the conditions of manufacture and storage and must be preserved against the contaminating action of microorganisms such as but not limited to bacteria and fungi.

Prevention of contamination by microorganisms can be achieved by but not limited to various antibacterial and antifungal agents known in the art. In many cases, it will be preferable to include isotonic agents, for example, sugars or polyalcohols such as mannitol, sorbitol or sodium chloride or sugars and polyalcohols and sodium chloride in the composition.

Prolonged absorption or controlled release or sustained release of the injectable embodiments of the present invention can be brought about by including in the composition, agents which delays absorption, including but not limited to; aluminum monostearate or gelatin or aluminum monostearate and gelatin.

The composition and other ingredients may also be enclosed in a hard shell or soft shell gelatin capsule, formed into ingestible tablets, formed into buccal tablets, formed into troches, mixed as elixirs, mixed as suspensions, mixed as syrups, formed into wafers, and the like or incorporated directly into the subject's diet.

Tablets, troches, pills, capsules and the like may also contain binders or excipients or lubricants or sweetening agents or binders and excipients and lubricants and sweetening agents. Various other materials may be present as coatings or to otherwise modify the physical form of the dosage unit. For instance, tablets, pills, or capsules may be coated with shellac, sugar, or both.

Depending on the route of administration, the compositions may be coated with a material to protect the compound from the action of acids and other natural conditions that may inactivate the compositions.

In order to administer the compositions transdermally or by injection, it may be necessary to coat the composition with, or co-administer the composition with, a material to prevent inactivation of the composition. For example, the composition may be administered to an individual in an appropriate diluent or in an appropriate carrier such as liposomes.

In yet another aspect, some embodiments of the invention provide a packaged pharmaceutical preparation is provided that contains a composition of the invention in a sealed container, with instructions for administration of the composition, typically self-administration. Generally, the packaged preparation contains a plurality of orally administrable unit dosage forms, and may feature each individual unit dosage form in a separate sealed housing, such as in a blister pack.

An aspect of the present invention features administering the exemplary embodiment of the invention metformin in combination with mifepristone.

In exemplary embodiments of the present invention metformin is administered at a daily dosage range of about 50 mg-3000 mg, including but not limited to, doses of 50 mg, 100 mg, 150 mg, 230 mg, 365 mg, 550 mg, 750 mg, 1150 mg, 1250 mg, 1560 mg, 1750 mg and 3000 mg daily.

Accordingly, in exemplary preferred embodiments of the present invention, mifepristone is prescribed at a dose of at least 5 mg to less than 1200 mg daily. In some embodiments of the invention the dose range of mifepristone is about 10 mg to about 800 mg daily, more preferably in other embodiments of the invention the dose range of mifepristone is about 20 mg to 600 mg daily, and optimally in yet other embodiments of the invention the dose range of mifepristone is about 30 mg to 400 mg daily, as noted above.

In another embodiment of the invention, the dosage of an active principal agent such as but not limited to metformin is increased gradually at the outset of the therapy in order to reduce the chance of undesirable effects of the composition or to enable a skilled artesian to assess therapeutic effectiveness or to reduce the chance of undesirable effects and to enable a skilled practitioner to assess therapeutic effectiveness. In an exemplary embodiment, the dose of the active principal metformin is 250 mg daily for about the first 7 days of treatment, for days 7-14 a dose of about 500 mg of metformin daily, for days 14-28 a dose of 1350 mg of metformin daily, for days 28 and beyond the dose of metformin is about 2500 mg daily. The skilled artesian is expected to make ongoing dosage adjustments pertinent to the health of the subject.

In an exemplary embodiment of the invention where mifepristone is an active principal agent, as it pertains to decreasing the dose of mifepristone or discontinuing the dose administration of mifepristone or decreasing the dose of mifepristone and discontinuing the dose administration of mifepristone, a tapered mifepristone dose reduction protocol may be employed, with or without concomitant alterations to the administration of other active principal agents.

Yet another embodiment of the present invention features pharmaceutical compositions for oral administration comprising metformin and mifepristone in a single pharmaceutical formulation. Such compositions may be preferred to increase patient compliance by reducing the number of dose administrations necessary to ensure the desired pharmacologic effect.

In some embodiments of the present invention, the pharmaceutical composition includes active principal agents in a controlled release formulation.

As defined herein, a “controlled release formulation” includes a pharmaceutical formulation that has been adapted such that active principal release rates and active principal release profiles can be matched to physiological requirements or chronotherapeutic requirements or physiological and chronotherapeutic requirements or alternatively, has been formulated to effect release of a drug at a programmed rate.

Controlled release formulations include, but are not limited to, granules, delayed release granules, hydrogels (e.g., of synthetic or natural origin), other gelling agents (e.g., gel-forming dietary fibers), matrix-based formulations (e.g., formulations comprising a polymeric material having at least one active ingredient dispersed there through), granules within a matrix, polymeric mixtures, granular masses, and the like.

In an embodiment of the invention, a controlled release formulation is a delayed release form. As defined herein, a “delayed release form” is formulated in such a way as to delay the onset of availability of an active principal agent to a subject to which it has been administered for an extended period of time.

A delayed release form may be formulated in such a way as to delay the release of an effective dose of an active principal agent for durations including but not limited to; 4, 8, 12, 16 or 24 hours following administration to a subject or following the release of another active principal agent comprising the composition or following administration to a subject and following the release of another active principal agent comprising the composition. In yet another preferred embodiment, a controlled release formulation is a sustained release form.

As defined herein, a “sustained release form” is formulated in such a way as to sustain the availability of an active principal agent to the biological system of a subject to which said active principal agent has been administered via a pharmaceutical composition of the invention over an extended period of time. A sustained release form can be formulated in such a way as to provide availability of an active principal agent to the biological system of a subject to which it has been administered for durations including but not limited to; 4, 8, 12, 16, 24 to 48 hours.

In yet another embodiment of the present invention, the pharmaceutical composition includes metformin in a controlled release formulation and further includes mifepristone in an immediate release formulation.

Exemplary compositions of the present invention include a tablet core containing the active principal agent metformin, said core being in association with a layer containing the active principal agent mifepristone.

In some exemplary embodiments of the invention the tablet core contains a delayed release form or sustained release form or delayed and sustained release form.

In another exemplary embodiment of the present invention, a tablet can comprise a first layer containing, for example, mifepristone in an immediate release formulation and a core containing, for example, metformin in a delayed release form or in a sustained release form or in a delayed release form and in a sustained release form.

Other exemplary embodiments of the invention may include, for example, a tablet with a barrier between the first layer and tablet core, said layer serving the purpose of limiting the release of the active principal agent from the surface of the core. Preferred barriers prevent dissolution of the core when the pharmaceutical formulation is first exposed to gastric fluid.

For example, a barrier may comprise a disintegrant, a dissolution-retarding coating (e.g., a polymeric material, for example, an enteric polymer), or a hydrophobic coating or film, or can be selectively soluble in either the stomach or intestinal fluids or may comprise a disintegrant and a dissolution-retarding coating and a hydrophobic coating or film and can be selectively soluble in either the stomach or intestinal fluids. Such barriers permit an active principal to leach out slowly and can cover substantially the whole surface of the core.

In some embodiments of the present invention pharmaceutical compositions are designed to release the two active principal agents of the of the present invention sequentially. In an exemplary embodiment of the present invention a pharmaceutical composition is formulated to enable releasing phenformin after releasing mifepristone, with both agents being contained in the same pharmaceutical composition unit dose form.

In an exemplary embodiment of the present invention a pharmaceutical composition contains the active principal agents of phenformin and mifepristone where the daily unit dosages range from about 5 mg to about 200 mg of phenformin and from about 25 mg to 400 mg of mifepristone.

Pharmaceutical compositions of the present invention may contain additional additives, suspending agents, diluents, binders or adjuvants, disintegrants, lubricants, glidants, stabilizers, coloring agents, flavoring agents, etc. These are conventional materials that may be incorporated in conventional amounts.

Some embodiments of the invention provide a packaged pharmaceutical preparation that contains a composition of the invention in which both active principal agents are provided in an immediate release form.

In yet another aspect, some embodiments of the invention provide a packaged pharmaceutical preparation that contains a composition of the invention in which one active principal agent is provided in an immediate release form, whereas the other active principal agent is provided in a sustained release form or controlled release form or sustained release form and controlled release form.

In another aspect, some embodiments of the invention provide a packaged pharmaceutical preparation that contains a composition of the invention in which both active principal agents are provided in a sustained release form or controlled release form or sustained release form and controlled release form.

In yet another aspect, some embodiments of the invention provide a packaged pharmaceutical preparation that contains a composition of the invention in which at least one active principal is present in both an immediate release form and a sustained release form or a controlled release form or a sustained release form and a controlled release form.

Yet another embodiment of the present invention features pharmaceutical compositions for oral administration comprising metformin and mifepristone administered in a single unit dose form or administered separately or administered in a single unit dose form and administered separately in combination with a chemical agent that enhances the composition's therapeutic effect.

This embodiment of the present invention is further illustrated by the following examples, which should not be construed as limiting. For example, long term treatment with the biguanide agents such as metformin is know to interfere with the gastrointestinal absorption of vitamin B12, contributing to vitamin B12 deficiencies in some subjects.

Therefore another embodiment of the present invention features pharmaceutical compositions comprising metformin, mifepristone and vitamin B12 administered in a single unit dose form or administered separately or administered in a single unit dose form and administered separately. Wherein the term vitamin B12 includes but is not limited to cyanocobalamin, methylcobalamin, hydroxocobalamin and related compounds.

In regard to embodiments of the present invention, the dose of an active principal agent may be administered at a rate of less than once per day. In other embodiments, the active principal agent is administered at least once per day. In yet other embodiments of the invention an active principal agent is administered in multiple doses, such as but not limited to, BID (e.g., twice daily), TID (three times daily) or QID (four times daily). Treatment with embodiments of the present invention when administered in neoplastic conditions are combined with additional chemical agents that demonstrate cytostatic effects or apoptotic lethality effects or cell cycle arrest effects or morphology change effects or inhibition of metastatic potential effects or reversal of multidrug resistance effects or cytostatic effects and apoptotic lethality effects and cell cycle arrest effects and morphology change effects and inhibition of metastatic potential effects and reversal of multidrug resistance effects.

Therefore, some embodiments of the present invention features pharmaceutical compositions comprising of at least two active principal agents where; at least one of the active principal agents is an inhibitor of a mitochondrial process known to generate ROS and at least one of the other active principals contributes to a reduced rate of mitochondrial oxygen consumption, such as but not limited to a pharmaceutical composition consisting of the active principal agents phenformin and mifepristone, in combination with additional agents useful in the treatment of neoplastic conditions, including but not limited to cytostatic agents, cytotoxic agents, anti-proliferative agents, aromatase inhibitors, hormone receptor antagonists, hormone receptor modulators, genetic inducers, genetic inhibitors, bisphosphonate agents in a single unit dose form or administered separately or in single unit dose form and administered separately.

In some embodiments of the present invention, the subject or patient is monitored about every 2-6 weeks, in other embodiments of the present invention, the subject or patient is monitored about every 3-5 and in yet other embodiments of the present invention, the subject or patient is monitored no sooner than every 6 weeks.

Monitoring the effectiveness of treatment with embodiments of the present invention to achieve therapeutic goals includes, but is not limited to monitoring the subject or patient's body weight, tissue or serum or plasma or tissue and serum and plasma biomarkers, radiological imaging studies, ultrasound imaging studies, magnetic resonance imaging studies.

Additional features of the subject or patient's health can also be monitored including, but not limited to the patient's blood pressure, heart rate, electroencephalography, electromyography, hepatic or other tissue elastography cognitive function. Likewise, monitoring a subject or patient for treatment associated side effects can include monitoring of at least one, preferably more than one know symptom associated with treatment.

The embodiments of the present invention demonstrate characteristic bioenergetic effects both in-vitro and in-vivo. Embodiments of the present invention such as the pharmaceutical composition [metformin/mifepristone] inhibit anaerobic metabolic activity in a eukaryotic biological system.

Furthermore, embodiments of the present invention demonstrate characteristic bioenergetic effects in vivo, including anti-inflammatory, improved insulin sensitivity, improved serum lipid profile and reduced oxidative stress that support the use of embodiments of the present invention as methods of treatment and/or prevention for diseases and/or disorders and/or conditions associated with bioenergetic dysfunction and/or oxidative stress.

The results of Experiment 1 reveal that the novel and unexpected characteristic bioenergetic effects demonstrated by the embodiments of the present invention are emergent properties of the embodiments that are non-additive and not the result of a generalized effect of a biguanide agent administered in association with a glucocorticoid receptor antagonist.

The glucocorticoid receptor antagonist properties of ketoconazole have long been established in the prior art (Loose D S, et al. Ketoconazole binds to glucocorticoid receptors and exhibits glucocorticoid antagonist activity in cultured cells. J Clin Invest. 1983 July; 72(1): 404-8).

The results of Experiment 1 demonstrate that C2C12 murine myoblasts treated with the exemplary embodiment of the present invention, [metformin/mifepristone] demonstrated no significantly greater level of anaerobic metabolic activity relative to the level of anaerobic metabolic activity demonstrated by untreated control condition C2C12 murine myoblasts (Table 27A1), whereas C2C12 murine myoblasts treated with the pharmaceutical composition [metformin/ketoconazole], which is a biguanide/glucocorticoid receptor antagonist combination, but not an embodiment of the present invention, demonstrated greater levels of anaerobic metabolic activity, to a statistically significant degree, relative to the level of anaerobic metabolic activity demonstrated by untreated control condition C2C12 murine myoblasts (Table 27F3).

Further, an exemplary embodiment of the present invention, [metformin/mifepristone], demonstrated the ability to inhibit anaerobic metabolic activity, while the pharmaceutical composition consisting of a biguanide agent and a glucocorticoid receptor antagonist combination, exemplified by [metformin/ketoconazole] demonstrated an inability to inhibit anaerobic metabolic activity.

Experiment 1 results demonstrated that anaerobic metabolic activity, as indicated by ECAR, in C2C12 murine myoblasts, under maximal aerobic metabolic conditions, treated with the pharmaceutical composition [metformin/mifepristone] was lower to a statistically significant degree relative to the level of anaerobic metabolic activity demonstrated by non-treated Control condition C2C12 murine myoblasts under maximal aerobic metabolic conditions (FCCP perturbed metabolism) (Table 27A7, Table 27B8).

While Experiment 1 results demonstrated that treatment of C2C12 murine myoblasts, under maximal aerobic metabolic conditions, with a pharmaceutical composition consisting of a biguinide agent and glucocorticoid receptor antagonist, [metformin/ketoconazole], demonstrated an inability to inhibit anaerobic metabolic activity relative to the level of anaerobic metabolic activity demonstrated by non-treated Control condition C2C12 murine myoblasts under maximal aerobic metabolic conditions (FCCP perturbed metabolism) (Table 27E1).

The contents of all references, patents, and published patent applications cited throughout this application are hereby incorporated by reference.

### EXPERIMENT 1

### Introduction

In the experiment, immortalized murine C2C12 myoblast cells were exposed to various pre-assay growth conditions prior to being seeded into a Seahorse XF24 Extracellular Flux Analyzer cell culture plate. The basal oxygen consumption (OCR) and extracellular acidification (ECAR) rates were measured to establish baseline metabolic rates for each experimental culture condition. The cells were then metabolically perturbed by the successive addition of three different compounds that shift the bioenergetic profile of the cell.

The first compound added following the collection of baseline metabolic data was oligomycin. Oligomycin inhibits ATP synthesis by blocking the proton channel of the Fo portion ATP synthase (Complex V). During methods of researching mitochondrial oxidative phosphorylation, oligomycin is used to prevent phosphorylating respiration.

When intact cells are exposed to oligomycin, it can be used to distinguish the percentage of O2 consumption devoted to ATP synthesis from the percentage of O2 consumption required in order to maintain mitochondrial membrane potential and overcome the natural proton leak that occurs across the inner mitochondrial membrane. Under such circumstances, the expected finding would be that cells exposed to oligomycin would demonstrate a decreased rate of oxygen consumption (decreased OCR) as a result of a decreased rate of ATP synthesis via mitochondrial oxidative phosphorylation. Correspondingly an increase in the extracellular acidification rate (ECAR) would be expected as the cell increases utilization of glycolysis as a source of ATP generation.

The second agent injected, carbonyl cyanide-p-trifluoromethoxyphenylhydrazone (FCCP), is an ionophore that is a mobile ion carrier. FCCP is an uncoupling agent, as it disrupts ATP synthesis by transporting hydrogen ions across the mitochondrial membrane instead of the proton channel of ATP synthase (Complex V).

This collapse of the mitochondrial membrane potential leads to a rapid consumption of energy and oxygen without the generation of ATP. In this case, the expected finding would be for both OCR and ECAR to increase, OCR due to uncoupling, and ECAR as the cells attempt to maintain their energy balance by using glycolysis to generate ATP.

FCCP treatment can be used to calculate the “spare” respiratory capacity of cells, defined as the quantitative difference between maximal uncontrolled OCR and the initial basal OCR. It has been proposed that the maintenance of some spare respiratory capacity even under conditions of maximal physiological or pathophysiological stimulus is a major factor defining the vitality and/or survivability of cells.

The ability of cells to respond to stress under conditions of increased energy demand is in large part influenced by the bioenergetic capacity of mitochondria. This bioenergetic capacity is determined by several factors, including the ability of the cell to deliver substrate to mitochondria and the functional capacity of enzymes involved in electron transport.

Rotenone, a Complex I inhibitor, is the third agent injected in sequence, it prevents the transfer of electrons from the Fe—S center in Complex I to ubiquinone (Coenzyme Q). The inhibition of Complex I prevents the potential energy in NADH from being converted to usable energy in the form of ATP.

Rotenone exposure inhibits mitochondrial respiration and enables both the mitochondrial and non-mitochondrial fractions contributing to respiration to be calculated. The expected finding under such circumstances would be a decrease in OCR due to impaired mitochondrial function, with a concomitant increase in ECAR as the cell shifts to a more glycolytic state in order to maintain its energy balance.

### Materials and Methods

Reagents and Materials: Oligomycin, FCCP, and Rotenone Solutions (Seahorse Mito Stress Test Kit), DMEM Running Media (Seahorse #100965-000), DMSO (Sigma D8418), Distilled Water (Gibco 15230-170), Calibration buffer (Seahorse Bioscience) Cells Injection, Immortalized murine C2C12 myoblast cells, Metformin (Sigma 1396309), Mifepristone (Sigma M8046), Ketoconazole (Sigma 1356508), FSB (Hyclone SH90070.30), Penn/Strep (Gibco 15140-122), Sodium Pyruvate (Sigma S8636), Glutamax (Gibco 35050-061), Growth Medium (500 ml DMEM, 10% FBS, 5 ml Penn/Strep, 5 ml Sodium Pyruvate, 5 ml Glutamax)

### Statistical Analysis

Statistical analysis was conducted via two-tailed Mann-Whitney U test of variance. U and P values were calculated using algorithms supplied by the Meta Numerics and ALGLIB statistical libraries. During calculations, when the value of N (the number of scores) was equal to or greater than 10, it was assumed that the sampling distribution was approximately normal, and a Z-ratio was also employed to calculate the value of P. The threshold for significance was set at P≦to 0.05 (Table 27A1 to Table 27F2).

### Experimental Protocol

C2C12 murine myoblast cells were placed into pre-assay growth condition categories and cultured for 24 hours.

XFAssay_8152014_146 consisted of CSC12 murine myoblast cells incubated at 37 degrees Centigrade under the following pre-assay conditions for 24 hours prior to undergoing extracellular flux analysis: control (C), metformin 1 mM (Met 1 mM), mifepristone 3 mM (Mife 3 mM) and a combination of metformin/mifepristone 1 mM/3 mM (Met/Mife 1 mM/3 mM).

XFAssay_8222014_853 consisted of CSC12 murine myoblast cells incubated at 37 degrees Centigrade under the following pre-assay conditions for 24 hours prior to undergoing extracellular flux analysis: control (C), metformin 1 mM (Met 1 mM), mifepristone 50 uM (Mife 50 uM) and a combination of metformin/mifepristone 1 mM/50 uM (Met/Mife 1 mM/50 uM).

XFAssay_10232014_839 consisted of CSC12 murine myoblast cells incubated at 37 degrees Centigrade under the following pre-assay conditions for 24 hours prior to undergoing extracellular flux analysis: control (C), metformin 25 uM (Met 25 uM), mifepristone 50 uM (Mife 50 uM) and a combination of metformin/mifepristone 25 uM/50 uM (Met/Mife 25 uM/50 uM).

XFAssay_712015_1658 consisted of CSC12 murine myoblast cells incubated at 37 degrees Centigrade under the following pre-assay conditions for 24 hours prior to undergoing extracellular flux analysis: control (C), ketoconazole 1 mM(KET 1 mM), ketoconazole 50 uM (KET 50 uM) and a combination of metformin/ketoconazole 1 mM/50 uM (MET/KET 1 mM/50 uM).

CSC12 murine myoblast cells were seeded into a Seahorse XF24 24 well culture plate at a density of 10,000 cells/well in 100 microliters of Growth Medium.

Metformin, mifepristone, metformin/mifepristone or ketoconazole, metformin/ketoconazole were added to experimental condition appropriate wells, in concentrations described. The seeded XF24 culture plates were placed into a 37 degree Centigrade incubator at 10% CO2 for 24 hours.

Oligomycin, FCCP and Rotenone solutions were prepared from the Seahorse Mito Stress Test Kit XF as follows using DMEM Running media: 10 uM Oligomycin, 30.0 uM FCCP, 20.0 μM Rotenone. These concentrations represent the 10× dilution that will be made when the compounds are injected into the well. The working concentrations are: 1 uM Oligomycin, 3.0 uM FCCP, 2.0 μM Rotenone

Using the XF prep station, the Growth Medium was replaced with DMEM running media, the final volume of medium was set to 160 μL per well.

The seeded XF24 culture plate was then placed into a 37 degree Centigrade incubator without CO2 for 60 minutes to allow cell cultures to pre-equilibrate with the assay medium.

The Oligomycin, FCCP and Rotenone solutions prepared in step 4, were warmed to 37 degrees Centigrade and loaded into the injector ports in the following manner: 16 microliters of Oligomycin solution was added to port A, 18 microliters of FCCP solution was added to port B and 20 microliters of Rotenone solution was added to port C.

Assay protocol commands were set in the following manner: Loop was set to three times for Basal, Oligomycin and FCCP conditions and 5 times for Rotenone conditions. Mix was set to three minutes, followed by a Rest period of two minutes and Measure was set to three minutes.

### EXPERIMENT 2

Oxidative stress may be characterized as a condition during which the generation of pro-oxidant species, including but not limited to: ROS and RNS, exceeds the reduction capacity of a system. Effects of oxidative stress can be observed at the molecular, organelle, cellular, tissue, organ and/or organism levels.

Oxidative stress and conditions, disorders and diseases related to oxidative stress are highly associated with cellular bioenergetic processes in eukaryotic organisms.

In order to determine the effects of the present invention on cellular bioenergetic processes, oxidative stress and oxidative stress-associated conditions in a human being, a human subject was treated with an exemplary preferred embodiment of the present invention, namely metformin/mifepristone.

The subject, an obese 34 year-old Caucasian male, after being screened and found free of serious cardiovascular and orthopedic conditions, was instructed on the technique for performing a two-handed kettlebell swing.

The subject was instructed to continue with his established exercise routine, which had been stable for the preceding six months and consisted of 4 to 5 yoga sessions per week, and an additional 2 to 4 exercise sessions per week, consisting of resistance and cardiovascular training.

The subject was instructed to maintain his present nutritional habits, avoiding any significant increase or decrease in total caloric intake, as well as, any significant alteration to the ratio of consumed macronutrients.

In addition to the maintenance of his established exercise routine and nutritional habits, the subject was instructed to conduct a familiarization routine for the two-handed kettlebell swing exercise consisting of 3-5 sets of 20 repetitions, with a weight of 15 to 30 pounds, twice weekly, for a period of six weeks.

Following the six-week familiarization period for the two-handed kettlebell swing exercise, the subject underwent a body composition analysis (Table 28A) utilizing an InBody 520 bioimpedance analyzer, manufactured by InBody Inc, and laboratory analysis on fasting-state blood (Table 28B) and urine (Table 28C) samples for markers of basal physiological status, oxidative stress and oxidative stress associated diseases, disorders and conditions.

Following collection of baseline biometric and laboratory data, the subject conducted an exercise to exhaustion test protocol utilizing the two-handed kettlebell swing. The subject was instructed to avoid strenuous physical exertion for 48 hours prior to the exercise to exhaustion test protocol.

Prior to the onset of the exercise to exhaustion test protocol, a resting blood lactate level was determined for the subject utilizing a Lactate Scout Plus blood lactate monitor, manufactured by EKF Diagnostics (Table 28D).

The subject initiated the exercise to exhaustion test protocol by performing a round of the two-handed kettlebell swing familiarization routine consisting of three sets of twenty repetitions of two-handed kettlebell swings with a 9.0 kg kettlebell.

The familiarization routine served to prepare the neuromuscular and cardiovascular systems for heavy exertion and also provided the opportunity to capture the measurements that defined the minimum superior and minimum inferior limit of travel for the kettelbell during the execution of a technically correct two-handed kettlebell swing (Table 28D).

Following completion of the familiarization routine the subject undertook ten minutes of passive recovery after which the subject engaged in the active phase of the exercise to exhaustion test protocol.

To initiate the active phase of the exercise to exhaustion test protocol the subject was instructed to perform as many two-handed kettlebell swings with a 24 kg kettlebell as possible with rest intervals taken ad libitum until the first occurrence of either; the point of perceived exhaustion rendering him unable to continue to perform, as defined by the subject, or the point when an inability to repeatably execute a technically correct two-handed kettelbell swing occurred (Table 28D).

At which point the subject was instructed to perform as many two-handed kettlebell swings with a 16 kg kettlebell with rest intervals taken ad libitum until the first occurrence of either; the point of perceived exhaustion, as defined by the subject, or the point when an inability to execute a technically correct two-handed kettlebell swing occurred (Table 28D).

At which point the subject was instructed to perform as many two-handed kettlebell swings with a 9.0 kg kettlebell with rest intervals taken ad libitum until the first occurrence of either; the point of perceived exhaustion, as defined by the subject, or the point when an inability to execute a technically correct two-handed kettlebell swing occurred (Table 28D).

At which point the subject was instructed to perform as many two-handed kettlebell swings with a 4.6 kg kettlebell with rest intervals taken ad libitum until the first occurrence of either; the point of perceived exhaustion, as defined by the subject, or the point when an inability to execute a technically correct two-handed kettlebell swing occurred (Table 28D).

At which point the subject was instructed to perform as many two-handed kettlebell swings with a 3.2 kg kettlebell with rest intervals taken ad libitum until the first occurrence of either; the point of perceived exhaustion, as defined by the subject, or the point when an inability to execute a technically correct two-handed kettlebell swing occurred (Table 28D).

The subject was instructed that he could elect to terminate the exercise to exhaustion test protocol at any point. A timer, in the form of a stopwatch, was started on the initiation of the subjects first attempt to perform a two-handed kettlebell swing with a 24 kg kettlebell and ran continuously until it was stopped at the termination of the exercise to exhaustion test protocol (Table 28D).

Only repetitions of the two-handed kettlebell swing that were executed to the technical specifications of the minimum superior and minimum inferior limits as previously defined were recorded. Repetitions meeting the minimum standards of execution for a two-handed kettlebell swing were recorded utilizing the tally counter feature of a stopwatch.

A blood lactate level was taken at 3 minutes and 5 minutes after the termination of the exercise to exhaustion test protocol. If the blood lactate level recorded 5 minutes after the termination of the exercise to exhaustion test protocol was found to be greater than or equal to the blood lactate level recorded 3 minutes after the termination of the exercise to exhaustion test protocol, a blood lactate reading would be recorded 7 minutes after termination of the exercise to exhaustion test protocol and every minute thereafter until a blood lactate level reading was recorded that was lower than the blood lactate level recorded 5 minutes after the termination of the exercise to exhaustion test protocol (Table 28D).

Utilizing the mass of the kettlebells, the number of repetitions at each respective mass, a doubling of the distance between the minimum superior and the minimum inferior limits (d) for a technically proficient two-handed kettlebell swing and time elapsed (t), values for work (W) and power (P) were generated (Table 28D).

Twenty-four hours after completion of the exercise to exhaustion test protocol the subject underwent laboratory analysis of a blood sample for biomarkers associated with oxidative stress (Table 28E).

Forty-eight hours after completion of the exercise to exhaustion test protocol the subject underwent laboratory analysis of a blood and urine sample for biomarkers associated with oxidative stress (Table 28F).

Following the exercise to exhaustion test protocol the subject was instructed to resume his previously established nutritional and exercise routines devoid of alterations for the next four weeks.

Four weeks following the exercise to exhaustion test protocol the subject was instructed to begin a 14-day course of treatment with the exemplary preferred embodiment of the present invention metformin/mifepristone. The subject was instructed to take one 500 mg tablet of metformin by mouth twice daily starting on day 1 and continuing taking one 500 mg tablet by mouth twice daily through day 14. The subject was instructed to take one 200 mg tablet of mifepristone once per day on days 2, 4, 6, 8, 10, 12 in conjunction with one of the two daily doses of 500 mg metformin tablets.

On day 15, following the 14-day course of treatment with the exemplary preferred embodiment of the present invention, metformin/mifepristone, the subject underwent post-treatment laboratory analysis of fasting blood (Table 28G) and urine (Table 28H) samples for markers of basal physiological status, oxidative stress and oxidative stress associated diseases, disorders and conditions.

On day 16, the subject underwent a body composition analysis (Table 28I) utilizing an InBody 520 bioimpedance analyzer, manufactured by InBody Inc, in addition to undergoing a repeat exercise to exhaustion test protocol utilizing the two-handed kettlebell swing.

The subject had been instructed to avoid strenuous physical exertion for 48-hours prior to participation in the exercise to exhaustion test protocol.

Prior to the onset of the exercise to exhaustion test protocol, a resting blood lactate level was determined for the subject utilizing a Lactate Scout Plus blood lactate monitor, manufactured by EKF Diagnostics (Table 28J).

The subject initiated the exercise to exhaustion test protocol by performing a round of the two-handed kettlebell swing familiarization routine consisting of three rounds of twenty repetitions of two-handed kettlebell swings with a 9.0 kg kettlebell.

The familiarization routine served to prepare the neuromuscular and cardiovascular systems for heavy exertion. The measurements that defined the minimum superior and minimum inferior limit of travel of the kettelbell during the execution of a technically correct two-handed kettlebell swing obtained during the baseline exercise to exhaustion test protocol were utilized as the limits for the post-metformin/mifepristone treatment exercise to exhaustion test protocol (Table 28J).

Following completion of the familiarization routine the subject undertook ten minutes of passive recovery after which the subject engaged in the active phase of the exercise to exhaustion test protocol.

During the active phase of the exercise to exhaustion test protocol the subject was instructed to perform as many two-handed kettlebell swings with a 24 kg kettlebell as possible with rest intervals taken ad libitum until the first occurrence of either; the point of perceived exhaustion, as defined by the subject, or the point when an inability to execute a technically correct two-handed kettelbell swing occurred (Table 28J).

At which point the subject was instructed to perform as many two-handed kettlebell swings with a 16 kg kettlebell with rest intervals taken ad libitum until the first occurrence of either; the point of perceived exhaustion, as defined by the subject, or the point when an inability to execute a technically correct two-handed kettlebell swing occurred (Table 28J).

At which point the subject was instructed to perform as many two-handed kettlebell swings with a 9.0 kg kettlebell with rest intervals taken ad libitum until the first occurrence of either; the point of perceived exhaustion, as defined by the subject, or the point when an inability to execute a technically correct two-handed kettlebell swing occurred (Table 28J).

At which point the subject was instructed to perform as many two-handed kettlebell swings with a 4.6 kg kettlebell with rest intervals taken ad libitum until the first occurrence of either; the point of perceived exhaustion, as defined by the subject, or the point when an inability to execute a technically correct two-handed kettlebell swing occurred (Table 28J).

At which point the subject was instructed to perform as many two-handed kettlebell swings with a 3.2 kg kettlebell with rest intervals taken ad libitum until the first occurrence of either; the point of perceived exhaustion, as defined by the subject, or the point when an inability to execute a technically correct two-handed kettlebell swing occurred (Table 28J).

The subject was instructed that they could elect to terminate the exercise to exhaustion test protocol at any point. A timer, in the form of a stopwatch, was started on the initiation of the subjects first attempt to perform a two-handed kettlebell swing with a 24 kg kettlebell and ran continuously until it was stopped at the termination of the exercise to exhaustion test protocol (Table 28J).

Only repetitions of the two-handed kettlebell swing that were executed to the technical specifications of the minimum superior and minimum inferior limits as previously defined were recorded. Repetitions meeting the minimum standards of execution for a two-handed kettlebell swing were recorded utilizing the tally counter feature of a stopwatch.

A blood lactate level was taken at 3 minutes and 5 minutes after the termination of the exercise to exhaustion test protocol. If the blood lactate level recorded 5 minutes after the termination of the exercise to exhaustion test protocol was found to be greater than or equal to the blood lactate level recorded 3 minutes after the termination of the exercise to exhaustion test protocol, a blood lactate reading would be recorded 7 minutes after termination of the exercise to exhaustion test protocol and every minute thereafter until a blood lactate level reading was recorded that was lower than the blood lactate level recorded 5 minutes after the termination of the exercise to exhaustion test protocol (Table 28J).

Utilizing the mass of the kettlebells, the number of repetitions at each respective mass, a doubling of the distance between the minimum superior and the minimum inferior limits (d) for a technically proficient two-handed kettlebell swing and time elapsed (t), values for work (W) and power (P) were generated (Table 28J).

Twenty-four hours after completion of the exercise to exhaustion test protocol the subject underwent laboratory analysis of a blood sample for biomarkers associated with oxidative stress (Table 28K).

Forty-eight hours after completion of the exercise to exhaustion test protocol the subject underwent laboratory analysis of a blood and urine sample for biomarkers associated with oxidative stress (Table 28F).

### Discussion

During the exit interview the test subject acknowledged performing a resistance training session less than 24 hours prior to the blood and urine sample collection for the post-treatment, pre-exercise stress test baseline laboratory evaluations. The subject reported mild gastrointestinal symptoms consisting of loose stools and indigestion during the first three days of treatment conditions, after which point symptoms resolved.

### Examples

The present invention is further described by means of the examples, presented below. The use of such examples is illustrative only and in no way limits the scope and meaning of the invention or of any exemplified term. Likewise, the invention is not limited to any particular preferred embodiments described herein. Indeed, many modifications and variations of the invention will be apparent to those skilled in the art upon reading this specification. The invention is therefore to be limited only by the terms of the appended claims along with the full scope of equivalents to which the claims are entitled.

Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric.

### Example 1

One example of a pharmaceutical formulation allowing for the controlled release of metformin and the immediate release of mifepristone is a controlled release metformin bead that can be made using an extrusion spheronization process to produce a matrix core comprised of metformin, about 40.0% w/w; microcrystalline cellulose (Avicel® PH102), about 56.5% w/w; and Methocel™ A15 LV, about 3.5% w/w. The metformin cores should be coated with ethyl cellulose, about 5.47% w/w, and Povidone K30, about 2.39% w/w.

The composition of the metformin matrix core so prepared would be as follows:

| Component | % w/w | Amount (mg) |
|---|---|---|
| Microcrystalline | 52.05 | 141.25 |
| cellulose,(Avicel ® PH102) | | |
| Metformin | 36.85 | 100.00 |
| Ethylcellulose, | 5.47 | 14.84 |
| Methylcellulose,(Methocel ™ | 3.22 | 8.74 |
| A15 LV | | |
| Polyvinylpyrrolidone, (Povidone | 2.39 | 6.49 |
| K30) | | |

Mifepristone is then coated onto sugar spheres to provide immediate release mifepristone beads. Both sets of beads are then encapsulated into each of a plurality of capsules, with each capsule containing 100 mg metformin (as metformin HCl) and 100 mg mifepristone.

### Example 2

Another pharmaceutical formulation allowing for the delivery of mifepristone (25 mg/5 ml) and metformin (100 mg/5 ml) as an oral liquid suspension. The oral liquid suspension formula would be comprised of metformin 2.0% w/v, mifepristone 0.25% w/v, colloidal silicone dioxide 0.40% w/v, erythritol solution 10.0% w/v, glycerin 25.0% w/v, sucrose 40.0% w/v, sodium methylparaben 0.15% w/v, xantham gum 0.28% w/v, peppermint flavor 0.25% w/v, citric acid monohydrate 0.06% w/v, simethicone emulsion(40%) 0.15% w/v, FD&C yellow #6 0.01% w/v, magnesium stearate 0.0018% w/v, purified water q.s. to 100%

Metformin 100 mg/Mifepristone 25 mg-5 ml

| Component | Component % w/v |
|---|---|
| Metformin | 2.0 |
| Mifepristone | 0.25 |
| Colloidal Silicon Dioxide | 0.40 |
| Glycerin | 25.0 |
| Sucrose | 40.0 |
| Sodium Methylparaben | 0.15 |
| Xantham Gum | 0.28 |
| Art Peppermint Flavor | 0.25 |
| Citric Acid Monohydrate | QS to adjust pH (0.06%) |
| Simethicone Emulsion (40%) | 0.15 |
| FD&C Yellow #6 | 0.01 |
| Erythritol Solution | 10.0 |
| Magnesium Stearate | 0.0018 |
| Purified Water | QS to 100% |

What is claimed is:

1) A pharmaceutical composition comprising of a first active principal and a second active principal;
a) wherein said first active principal is an inhibitor of a mitochondrial reactive oxygen species (ROS) generating process; and
b) wherein said second active principal contributes to a reduced rate of mitochondrial oxygen consumption; and
c) wherein said pharmaceutical composition inhibits anaerobic metabolic activity when administered to a eukaryotic biological system.

2) The pharmaceutical composition of claim 1;
a) wherein said inhibitor of a mitochondrial ROS generating process inhibits the activity of the enzyme mitochondrial NADH-coenzyme Q oxidoreductase (Complex I); and
b) wherein said active principal that contributes to a reduced rate of mitochondrial oxygen consumption comprises of mifepristone.

3) The pharmaceutical composition of claim 2, wherein said inhibitor of mitochondrial NADH-coenzyme Q oxidoreductase (Complex I) comprises of a biguanide.

4) The pharmaceutical composition of claim 3, wherein said biguanide is selected from the group consisting of metformin, phenformin and buformin.

5) The pharmaceutical composition of claim 1;
a) wherein said first active principal comprises of a therapeutically effective amount of a biguanide; and
b) wherein said second active principal comprises of a therapeutically effective amount of mifepristone.

6) The pharmaceutical composition of claim 5;
a) wherein said first active principal comprises of a therapeutically effective amount of metformin; and
b) said second active principal comprises of a therapeutically effective amount of mifepristone.

7) The pharmaceutical composition of claim 5;
a) wherein said first active principal comprises of a therapeutically effective amount of phenformin; and
b) wherein said second active principal comprises of a therapeutically effective amount of mifepristone.

8) The pharmaceutical composition of claim 5;
a) wherein said first active principal comprises of a therapeutically effective amount of buformin; and
b) wherein said second active principal comprises of a therapeutically effective amount of mifepristone.

9) The pharmaceutical composition of claim 6;
a) wherein said therapeutically effective amount of metformin is from 50 to 3000 mg daily; and
b) wherein said therapeutically effective amount of mifepristone is from 5 to 1200 mg daily

10) The pharmaceutical composition of claim 7;
a) wherein said therapeutically effective amount of phenformin is from 5 to 500 mg daily; and
b) wherein said therapeutically effective amount of mifepristone is from 5 to 1200 mg daily.

11) The pharmaceutical composition of claim 8;
a) wherein said therapeutically effective amount of buformin is from 5 to 1500 mg daily; and
b) wherein said therapeutically effective amount of mifepristone is from 5 to 1200 mg daily.

12) A method for treatment of diseases and/or disorders and/or conditions associated with bio-energetic dysfunction, comprising of:
administering to an animal a therapeutically effective amount of a pharmaceutical composition comprising of a first active principal and a second active principal,
a) wherein said first active principal is an inhibitor of a mitochondrial reactive oxygen species (ROS) generating process;
b) wherein said second active principal contributes to a reduced rate of mitochondrial oxygen consumption; and
c) wherein said pharmaceutical composition inhibits anaerobic metabolic activity when administered to a eukaryotic biological system.

13) The method of claim 12, wherein said pharmaceutical composition is administered in an oral dosage form;
a) wherein said pharmaceutical composition contains at least one active principal administered in a sustained release oral dosage form;
b) wherein said pharmaceutical composition contains at least one active principal administered in an immediate release oral dosage form;
c) wherein said pharmaceutical composition is administered in an oral dosage form selected from the group consisting of: a hard shell capsule, a soft shell capsule, tablets, buccal tablets, troches, elixirs, suspensions, syrups, wafers, and
d) wherein said pharmaceutical composition is incorporated directly into the subject's diet.

14) The method of claim 13, wherein said first active principal and said second active principal are administered simultaneously.

15) The method of claim 13, wherein said first active principal and said second active principal are administered separately.

16) The method of claim 13, wherein said animal is a human.

17) The method of claim 13, wherein the first active principal comprises of metformin and the second active principal comprises of mifepristone.

18) The method of claim 17, wherein said therapeutically effective amount of metformin is from 50 to 3000 mg daily and said therapeutically effective amount of mifepristone is from 5 to 1200 mg daily concentration.
